(19) United States
(12) Patent Application Publication
(10) Pub. No.: US 2015/0339817 A1
(43) Pub. Date: Nov. 26, 2015

(54) ENDOSCOPE IMAGE PROCESSING DEVICE, ENDOSCOPE APPARATUS, IMAGE PROCESSING METHOD, AND INFORMATION STORAGE DEVICE

(71) Applicant: OLYMPUS CORPORATION, Tokyo (JP)

(72) Inventor: Naoya KURIYAMA, Tokyo (JP)

(73) Assignee: OLYMPUS CORPORATION, Tokyo (JP)

(21) Appl. No.: 14/813,618

(22) Filed: Jul. 30, 2015

Related U.S. Application Data
(63) Continuation of application No. PCT/JP2013/077286, filed on Oct. 8, 2013.

(30) Foreign Application Priority Data
Jan. 31, 2013 (JP) 2013-016464
Apr. 3, 2013 (JP) 2013-077613

(51) Int. Cl. G06T 7/00; G06K 9/46; G06K 9/62; G01B 11/14; G06K 9/52; A61B 1/04; G06T 5/00

(52) U.S. Cl. CPC G06T 7/0012; A61B 1/04; G06K 9/46; G06T 5/00; G01B 11/14; G06K 9/468; G06K 9/4652; G06K 9/52; G06K 9/6215; G06K 9/4604; G06K 9/6267; G06K 2009/4666

(57) ABSTRACT

An endoscope image processing device includes an image acquisition section, a distance information acquisition section, a concavity-convexity determination section that performs a concavity-convexity determination process that determines a concavity-convexity part of an object that agrees with characteristics specified by known characteristic information based on distance information and the known characteristic information that represents known characteristics relating to a structure of the object, a mucous membrane determination section that determines a mucous membrane area within the captured image, and an enhancement processing section that performs an enhancement process on the mucous membrane area based on information about the concavity-convexity part determined by the concavity-convexity determination process. The concavity-convexity determination section excludes a structure that is more global than a local concavity-convexity structure having a desired size from the distance information based on the known characteristic information to extract the local concavity-convexity structure having the desired size as the concavity-convexity part.

### CROSS REFERENCE TO RELATED APPLICATION

This application is a continuation of International Patent Application No. PCT/JP2013/077286, having an international filing date of Oct. 8, 2013, which designated the United States, the entirety of which is incorporated herein by reference. Japanese Patent Application No. 2013-016464 filed on Jan. 31, 2013 and Japanese Patent Application No. 2013-077613 filed on Apr. 3, 2013 are also incorporated herein by reference in their entirety.

### BACKGROUND

The present invention relates to an endoscope image processing device, an endoscope apparatus, an image processing method, an information storage device, and the like.

When observing tissue using an endoscope apparatus, and making a diagnosis, a method has been widely used that determines whether or not an early lesion has occurred by observing tissue as to the presence or absence of minute concavities-convexities (concavity-convexity parts). When using an industrial endoscope apparatus instead of a medical endoscope apparatus, it is useful to observe the object (i.e., the surface of the object in a narrow sense) as to the presence or absence of a concavity-convexity structure in order to detect whether or not a crack has occurred in the inner side of a pipe that is difficult to directly observe with the naked eye, for example. It is also normally useful to detect the presence or absence of a concavity-convexity structure from the processing target image (object) when using an image processing device other than an endoscope apparatus.

For example, a method that performs image processing that enhances a specific spatial frequency, and the method disclosed in JP-A-2003-88498, have been known as a method that enhances a structure (e.g., concavity-convexity structure such as a groove) within the captured image by image processing. A method that effects some change in the object (e.g., dye spraying), and captures the object has also been known. JP-A-2003-88498 discloses a method that enhances a concavity-convexity structure by comparing the luminance level of an attention pixel in a locally extracted area with the luminance level of its peripheral pixel, and coloring the attention area when the attention area is darker than the peripheral area.

### SUMMARY

According to one aspect of the invention, there is provided an endoscope image processing device comprising:

an image acquisition section that acquires a captured image that includes an image of an object;

a distance information acquisition section that acquires distance information based on a distance from an imaging section to the object when the imaging section captured the captured image;

a concavity-convexity determination section that performs a concavity-convexity determination process based on the distance information, and known characteristic information that represents known characteristics relating to a structure of the object, the concavity-convexity determination process determining a concavity-convexity part of the object that agrees with the characteristics specified by the known characteristic information;

a mucous membrane determination section that determines a mucous membrane area within the captured image, the mucous membrane area being an area of a mucous membrane; and

an enhancement processing section that performs an enhancement process on the mucous membrane area determined by the mucous membrane determination section based on information about the concavity-convexity part determined by the concavity-convexity determination process,

the concavity-convexity determination section excluding a structure that is more global than a local concavity-convexity structure having a desired size from the distance information based on the known characteristic information to extract the local concavity-convexity structure having the desired size as the concavity-convexity part.

According to another aspect of the invention, there is provided an endoscope image processing device comprising:

an image acquisition section that acquires a captured image that includes an image of an object;

a distance information acquisition section that acquires distance information based on a distance from an imaging section to the object when the imaging section captured the captured image;

a concavity-convexity determination section that performs a concavity-convexity determination process based on the distance information, and known characteristic information that represents known characteristics relating to a structure of the object, the concavity-convexity determination process determining a concavity-convexity part of the object that agrees with the characteristics specified by the known characteristic information;

an exclusion target determination section that determines an exclusion target area within the captured image, the exclusion target area being an area of an exclusion target; and

an enhancement processing section that performs an enhancement process on the captured image based on information about the concavity-convexity part determined by the concavity-convexity determination process, while omitting or suppressing the enhancement process on the exclusion target area determined by the exclusion target determination section,

the concavity-convexity determination section excluding a structure that is more global than a local concavity-convexity structure having a desired size from the distance information based on the known characteristic information to extract the local concavity-convexity structure having the desired size as the concavity-convexity part.

According to another aspect of the invention, there is provided an endoscope apparatus comprising one of the above endoscope image processing devices.

According to another aspect of the invention, there is provided an image processing method comprising:

acquiring a captured image that includes an image of an object;

acquiring distance information based on a distance from an imaging section to the object when the imaging section captured the captured image;

performing a concavity-convexity determination process that excludes a structure that is more global than a local concavity-convexity structure having a desired size from the distance information based on known characteristic information to extract the local concavity-convexity structure having the desired size as a concavity-convexity part of the object that agrees with characteristics specified by the known characteristic information, to determine the concavity-convexity part, the known characteristic information being information that represents known characteristics relating to a structure of the object;

determining a mucous membrane area within the captured image, the mucous membrane area being an area of a mucous membrane; and

performing an enhancement process on the determined mucous membrane area based on information about the concavity-convexity part determined by the concavity-convexity determination process.

According to another aspect of the invention, there is provided an image processing method comprising:

acquiring a captured image that includes an image of an object;

acquiring distance information based on a distance from an imaging section to the object when the imaging section captured the captured image;

performing a concavity-convexity determination process that excludes a structure that is more global than a local concavity-convexity structure having a desired size from the distance information based on known characteristic information to extract the local concavity-convexity structure having the desired size as a concavity-convexity part of the object that agrees with characteristics specified by the known characteristic information, to determine the concavity-convexity part, the known characteristic information being information that represents known characteristics relating to a structure of the object;

determining an exclusion target area within the captured image, the exclusion target area being an area of an exclusion target; and

performing an enhancement process on the captured image based on information about the concavity-convexity part determined by the concavity-convexity determination process, while omitting or suppressing the enhancement process on the determined exclusion target area.

According to another aspect of the invention, there is provided a non-transitory information storage device storing an image processing program that causes a computer to perform steps of:

acquiring a captured image that includes an image of an object;

acquiring distance information based on a distance from an imaging section to the object when the imaging section captured the captured image;

performing a concavity-convexity determination process that excludes a structure that is more global than a local concavity-convexity structure having a desired size from the distance information based on known characteristic information to extract the local concavity-convexity structure having the desired size as a concavity-convexity part of the object that agrees with characteristics specified by the known characteristic information, to determine the concavity-convexity part, the known characteristic information being information that represents known characteristics relating to a structure of the object;

determining a mucous membrane area within the captured image, the mucous membrane area being an area of a mucous membrane; and

performing an enhancement process on the determined mucous membrane area based on information about the concavity-convexity part determined by the concavity-convexity determination process.

According to another aspect of the invention, there is provided a non-transitory information storage device storing an image processing program that causes a computer to perform steps of:

acquiring a captured image that includes an image of an object;

acquiring distance information based on a distance from an imaging section to the object when the imaging section captured the captured image;

performing a concavity-convexity determination process that excludes a structure that is more global than a local concavity-convexity structure having a desired size from the distance information based on known characteristic information to extract the local concavity-convexity structure having the desired size as a concavity-convexity part of the object that agrees with characteristics specified by the known characteristic information, to determine the concavity-convexity part, the known characteristic information being information that represents known characteristics relating to a structure of the object;

determining an exclusion target area within the captured image, the exclusion target area being an area of an exclusion target; and

performing an enhancement process on the captured image based on information about the concavity-convexity part determined by the concavity-convexity determination process, while omitting or suppressing the enhancement process on the determined exclusion target area.

### BRIEF DESCRIPTION OF THE DRAWINGS

FIG. 1 illustrates a first configuration example of an image processing device.

FIG. 2 illustrates a second configuration example of an image processing device.

FIG. 3 illustrates a configuration example of an endoscope apparatus according to the first embodiment.

FIG. 4 illustrates a detailed configuration example of a rotary color filter.

FIG. 5 illustrates a detailed configuration example of an image processing section according to the first embodiment.

FIG. 6 illustrates a detailed configuration example of a mucous membrane determination section.

FIGS. 7A and 7B are views illustrating an enhancement level used for an enhancement process.

FIG. 8 illustrates a detailed configuration example of a concavity-convexity information acquisition section.

FIGS. 9A to 9F are views illustrating a process that extracts extracted concavity-convexity information using a morphological process.

FIGS. 10A to 10D are views illustrating a process that extracts extracted concavity-convexity information using a filtering process.

FIG. 11 illustrates a detailed configuration example of a mucous membrane concavity-convexity determination section and an enhancement processing section.

FIG. 12 illustrates an example of extracted concavity-convexity information.

FIG. 13 is a view illustrating a concavity width calculation process.

FIG. 14 is a view illustrating a concavity depth calculation process.

FIGS. 15A and 15B are views illustrating an enhancement level (gain coefficient) setting example when performing an enhancement process on a concavity.

FIG. 16 illustrates a detailed configuration example of a distance information acquisition section.

FIG. 17 illustrates a detailed configuration example of an image processing section according to the second embodiment.

FIG. 18 illustrates a detailed configuration example of an exclusion target determination section.

FIG. 19 illustrates a detailed configuration example of an exclusion target object determination section.

FIG. 20 illustrates an example of a captured image after insertion of forceps.

FIGS. 21A to 21C are views illustrating an exclusion target determination process when a treatment tool is the exclusion target.

FIG. 22 illustrates a detailed configuration example of an exclusion target scene determination section.

FIG. 23 illustrates a detailed configuration example of an image processing section according to the third embodiment.

FIG. 24A illustrates the relationship between an imaging section and an object when observing an abnormal area, and FIG. 24B illustrates an example of an acquired image.

FIG. 25 is a view illustrating a classification process.

FIG. 26 illustrates a detailed configuration example of a mucous membrane determination section according to the third embodiment.

FIG. 27 illustrates a detailed configuration example of an image processing section according to the first modification of the third embodiment.

FIG. 28 illustrates a detailed configuration example of an image processing section according to the second modification of the third embodiment.

FIG. 29 illustrates a detailed configuration example of an image processing section according to the fourth embodiment.

FIG. 30 illustrates a detailed configuration example of a concavity-convexity determination section (third and fourth embodiments).

FIGS. 31A and 31B are views illustrating a process performed by a surface shape calculation section.

FIG. 32A illustrates an example of a basic pit, and FIG. 32B illustrates an example of a corrected pit.

FIG. 33 illustrates a detailed configuration example of a surface shape calculation section.

FIG. 34 illustrates a detailed configuration example of a classification processing section when implementing a first classification method.

FIGS. 35A to 35F are views illustrating a specific example of a classification process.

FIG. 36 illustrates a detailed configuration example of a classification processing section when implementing a second classification method.

FIG. 37 illustrates an example of a classification type when using a plurality of classification types.

FIGS. 38A to 38F illustrate an example of a pit pattern.

### DESCRIPTION OF EXEMPLARY EMBODIMENTS

Exemplary embodiments of the invention are described below. Note that the exemplary embodiments described below do not in any way limit the scope of the invention laid out in the claims. Note also that all of the elements described below in connection with the exemplary embodiments should not necessarily be taken as essential elements of the invention.

### 1. Method

A method that effects some change in the object, and captures the object has been known as a method that enhances concavities-convexities of the object. For example, when using a medical endoscope apparatus, the contrast of the mucous membrane in the surface area may be increased by spraying a dye (e.g., indigo carmine) to stain the tissue. However, it takes time and cost to spray a dye, and the original color of the object, or the visibility of a structure other than concavities-convexities, may be impaired due to the sprayed dye. Moreover, the method that sprays a dye to tissue is highly invasive for the patient.

In order to deal with these problems, several embodiments of the invention enhance concavities-convexities of the object by image processing. Note that concavity-convexity parts may be classified, and an enhancement process may be performed corresponding to the classification results. The enhancement process may be implemented using various methods, such as a method that simulates dye spraying, or a method that enhances a high-frequency component. However, when concavities-convexities of the object are enhanced by image processing, concavities-convexities of the object that should not be enhanced are also enhanced in the same manner as concavities-convexities of the object that should be enhanced.

For example, since concavities-convexities of a mucous membrane that should be enhanced and concavities-convexities of a treatment tool or the like that need not be enhanced are enhanced, the effect of the enhancement process that improves the accuracy of detection of an early lesion present on a mucous membrane is limited.

An object that should be enhanced is not present within the image in a specific scene (e.g., when water is supplied, or when mist is produced). In this case, since the user observes an image that is unnecessarily enhanced, the user may get tired as compared with the case where the enhancement process is not performed.

According to several embodiments of the invention, when a mucous membrane (i.e., an object that should be enhanced) is included within the image, the enhancement process is performed on the object that should be enhanced. When the image captures an object (or a scene) that should not be enhanced, the enhancement process on the object (or the entire image) is omitted or suppressed.

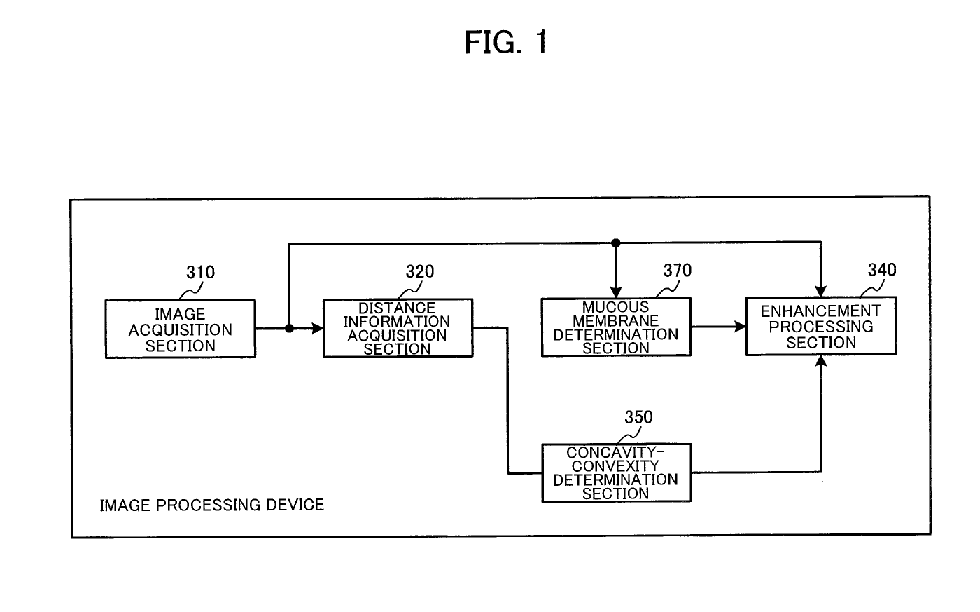

FIG. 1 illustrates a first configuration example of an image processing device as a configuration example when the enhancement process is performed on the object that should be enhanced. The image processing device includes an image acquisition section 310, a distance information acquisition section 320, a concavity-convexity determination section 350, a mucous membrane determination section 370, and an enhancement processing section 340.

The image acquisition section 310 acquires a captured image that includes an image of the object. The distance information acquisition section 320 acquires distance information based on the distance from an imaging section to the object when the imaging section captured the captured image. The concavity-convexity determination section 350 performs a concavity-convexity determination process that determines a concavity-convexity part of the object that agrees with the characteristics specified by known characteristic information based on the distance information and the known characteristic information, the known characteristic information being information that represents known characteristics relating to the structure of the object. The mucous membrane determination section 370 determines a mucous membrane area within the captured image. The enhancement processing section 340 performs an enhancement process on the determined mucous membrane area based on information about the concavity-convexity part determined by the concavity-convexity determination process.

This configuration example makes it possible to determine a mucous membrane (i.e., an object that should be enhanced), and perform the enhancement process on the determined mucous membrane. Specifically, it is possible to perform the enhancement process on the mucous membrane while omitting or suppressing the enhancement process on an area other than the mucous membrane that need not be enhanced. This makes it possible for the user to easily discriminate between the mucous membrane and an area other than the mucous membrane, and makes it possible to improve the examination accuracy, and reduce the degree to which the user gets tired.

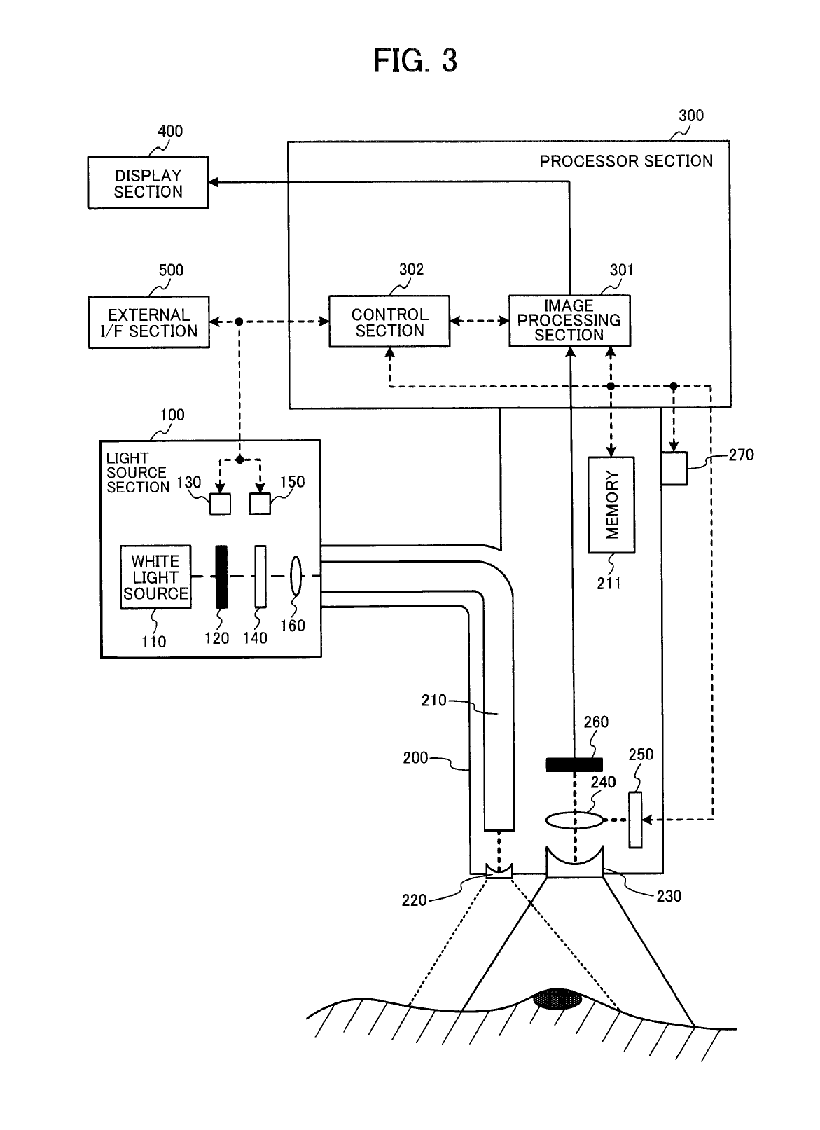

The term “distance information” used herein refers to information in which each position of the captured image is linked to the distance to the object at each position of the captured image. For example, the distance information is a distance map. The term “distance map” used herein refers to a map in which the distance (depth) to the object in the Z-axis direction (i.e., the direction of the optical axis of the imaging section 200 illustrated in FIG. 3) is specified corresponding to each point (e.g., each pixel) in the XY plane, for example.

Note that the distance information may be various types of information that are acquired based on the distance from the imaging section 200 to the object. For example, when implementing triangulation using a stereo optical system, the distance with respect to an arbitrary point of a plane that connects two lenses that produce a parallax may be used as the distance information. When using a Time-of-Flight method, the distance with respect to each pixel position in the plane of the image sensor may be acquired as the distance information, for example. In such a case, the distance measurement reference point is set to the imaging section 200. Note that the distance measurement reference point may be set to an arbitrary position other than the imaging section 200, such as an arbitrary position within the three-dimensional space that includes the imaging section and the object. The distance information acquired using such a reference point is also intended to be included within the term “distance information”.

The distance from the imaging section 200 to the object may be the distance from the imaging section 200 to the object in the depth direction, for example. For example, the distance in the direction of the optical axis of the imaging section 200 may be used. For example, when a viewpoint is set in the direction orthogonal to the optical axis of the imaging section 200, the distance from the imaging section 200 to the object may be the distance observed at the viewpoint (i.e., the distance from the imaging section 200 to the object along a line that passes through the viewpoint and is parallel to the optical axis).

For example, the distance information acquisition section 320 may transform the coordinates of each corresponding point in a first coordinate system in which a first reference point of the imaging section 200 is the origin, into the coordinates of each corresponding point in a second coordinate system in which a second reference point within the three-dimensional space is the origin, using a known coordinate transformation process, and measure the distance based on the coordinates obtained by transformation. In this case, the distance from the second reference point to each corresponding point in the second coordinate system is identical with the distance from the first reference point to each corresponding point in the first coordinate system (i.e., the distance from the imaging section to each corresponding point).

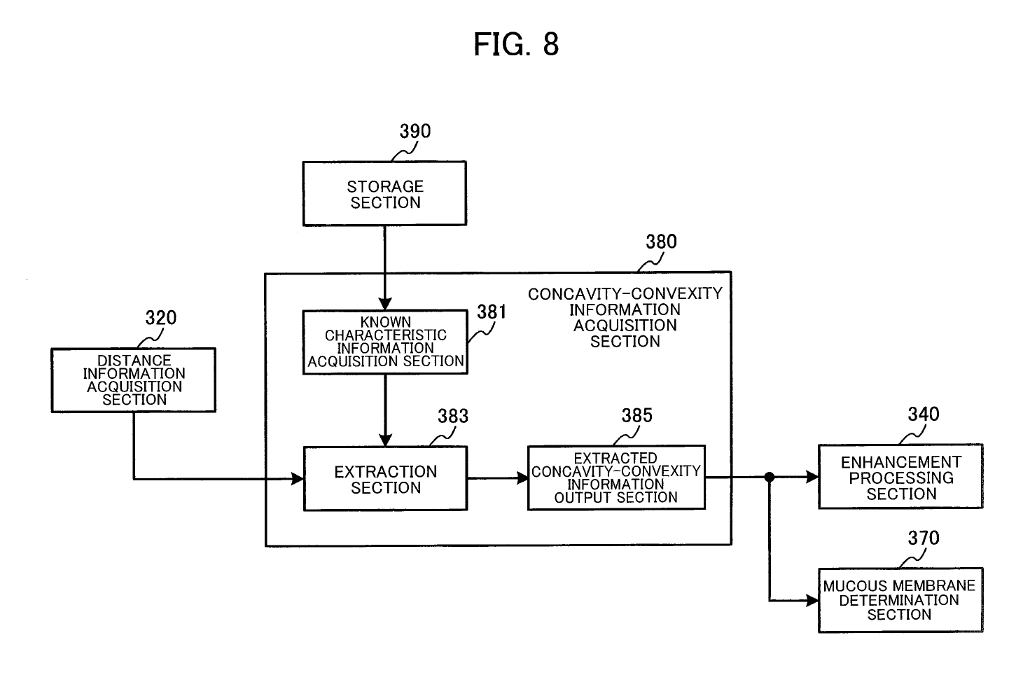

The distance information acquisition section 320 may set a virtual reference point at a position that can maintain a relationship similar to the relationship between the distance values of the pixels on the distance map acquired when setting the reference point to the imaging section 200, to acquire the distance information based on the distance from the imaging section 200 to each corresponding point. For example, when the actual distances from the imaging section 200 to three corresponding points are “3”, “4”, and “5”, respectively, the distance information acquisition section 320 may acquire distance information “1.5”, “2”, and “2.5” respectively obtained by halving the actual distances “3”, “4”, and “5” while maintaining the relationship between the distance values of the pixels. When the concavity-convexity information acquisition section 380 acquires the concavity-convexity information using the extraction operation parameter (as described later with reference to FIG. 8 and the like), the concavity-convexity information acquisition section 380 uses a different extraction process parameter as compared with the case where the reference point is set to the imaging section 200. Since it is necessary to use the distance information when determining the extraction process parameter, the extraction process parameter is determined in a different way when the distance measurement reference point has changed (i.e., when the distance information is represented in a different way). For example, when extracting the extracted concavity-convexity information using a morphological process (described later), the size of a structural element (e.g., the diameter of a sphere) used for the extraction process is adjusted, and the concavity-convexity part extraction process is performed using the structural element that has been adjusted in size.

The term “known characteristic information” used herein refers to information by which a useful structure of the surface of the object can be distinguished from an unuseful structure of the surface of the object. Specifically, the known characteristic information may be information about a concavity-convexity part for which the enhancement process is useful (e.g., a concavity-convexity part that is useful for finding an early lesion). In this case, an object that agrees with the known characteristic information is determined to be the enhancement target. Alternatively, the known characteristic information may be information about a structure for which the enhancement process is not useful. In this case, an object that does not agree with the known characteristic information is determined to be the enhancement target. Alternatively, information about a useful concavity-convexity part and information about an unuseful structure may be stored, and the range of the useful concavity-convexity part may be set with high accuracy.

The known characteristic information may be information that makes it possible to classify the structures of the object into specific types or states. For example, the known characteristic information may be information for classifying the structures of tissue into a blood vessel, a polyp, a cancer, another lesion, and the like, and may be information about the shape, the color, the size, and the like that are specific to such a structure. The known characteristic information may be information by which whether a specific structure (e.g., a pit pattern observed on the mucous membrane of the large intestine) is normal or abnormal can be determined, and may be information about the shape, the color, the size, and the like of the normal or abnormal structure.

The entire mucous membrane captured within the captured image may be determined to be a mucous membrane area, or part of the mucous membrane captured within the captured image may be determined to be a mucous membrane area. Specifically, it suffices that an area of the mucous membrane that is subjected to the enhancement process be determined to be a mucous membrane area. For example, a groove formed in the surface of tissue may be determined to be a mucous membrane area, and subjected to the enhancement process (described later). An area of the surface of tissue other than concavities-convexities for which the feature quantity (e.g., color) satisfies a given condition, may be determined to be a mucous membrane area.

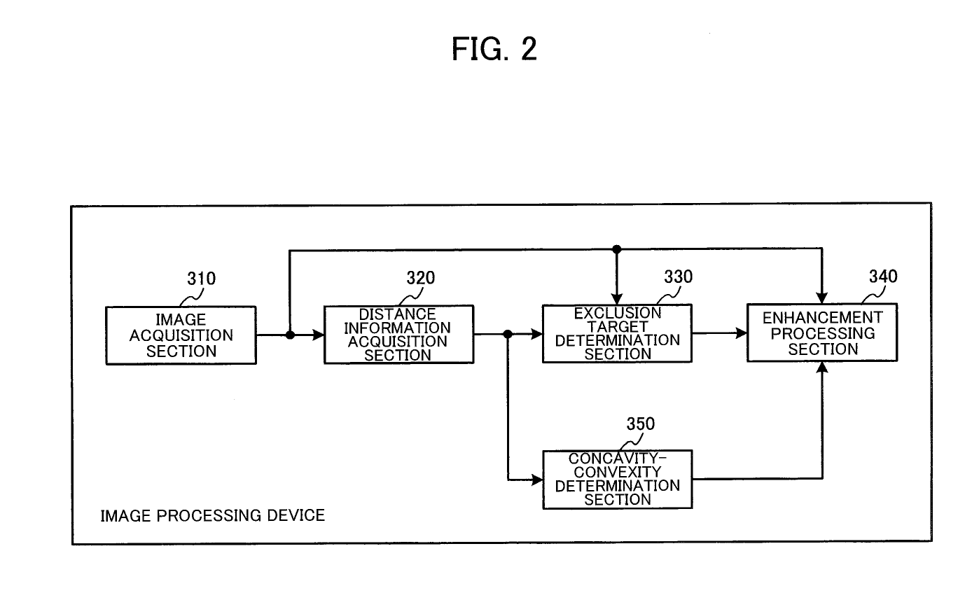

FIG. 2 illustrates a second configuration example of an image processing device as a configuration example when the enhancement process on the object (or the scene) that should not be enhanced is omitted or suppressed. The image processing device includes an image acquisition section 310, a distance information acquisition section 320, a concavity-convexity information acquisition section 380, an exclusion target determination section 330, and an enhancement processing section 340.

The image acquisition section 310 acquires a captured image that includes an image of the object. The distance information acquisition section 320 acquires distance information based on the distance from an imaging section to the object when the imaging section captured the captured image. The concavity-convexity determination section 350 performs a concavity-convexity determination process that determines a concavity-convexity part of the object that agrees with the characteristics specified by known characteristic information based on the distance information and the known characteristic information, the known characteristic information being information that represents known characteristics relating to the structure of the object. The enhancement processing section 340 performs an enhancement process on the captured image based on information about the concavity-convexity part determined by the concavity-convexity determination process. The exclusion target determination section 330 determines the exclusion target area within the captured image that is not subjected to the enhancement process. In this case, the enhancement processing section 340 omits or suppresses the enhancement process on the determined exclusion target area.

This configuration example makes it possible to determine an object that should not be enhanced, and omit or suppress the enhancement process on the determined object. Specifically, it is possible to perform the enhancement process on an area other than the exclusion target area (i.e., perform the enhancement process on a mucous membrane that should be enhanced). This makes it possible for the user to easily discriminate between the mucous membrane and an area other than the mucous membrane, and makes it possible to improve the examination accuracy, and reduce the degree to which the user gets tired.

The term “exclusion target” used herein refers to an object (e.g., other than tissue) or a scene that need not be enhanced, or an object or a scene for which the enhancement process is unuseful (e.g., an object or a scene that may hinder a doctor's medical examination when enhanced). Examples of the exclusion target include an object such as a residue, a bleeding area, a treatment tool, a blocked-up shadow area, and a blown-out highlight area, and a specific scene such as a water supply scene and an IT knife treatment scene. For example, when a treatment using an IT knife is performed, mist is produced when tissue is cauterized using the knife. An image that is difficult to observe may be obtained when the enhancement process is performed on an image in which mist is captured. Therefore, when the exclusion target object is captured within the captured image, the enhancement process on that area is omitted (or suppressed). When the captured image captures the exclusion target scene, the enhancement process on the entire captured image is omitted (or suppressed).

### 2. First Embodiment

### 2.1. Endoscope Apparatus

A detailed embodiment to which the above image processing device is applied is described below. A first embodiment illustrates an example in which a process that extracts a local concavity-convexity structure (e.g., polyp or folds) having the desired size (e.g., width, height, or depth) while excluding a global structure (e.g., surface undulations that are larger than folds) that is larger than the local concavity-convexity structure is performed as a process that determines a concavity-convexity part of the object.

FIG. 3 illustrates a configuration example of an endoscope apparatus according to the first embodiment. The endoscope apparatus includes a light source section 100, an imaging section 200, a processor section 300, a display section 400, and an external I/F section 500.

The light source section 100 includes a white light source 110, a light source aperture 120, a light source aperture driver section 130 that drives the light source aperture 120, and a rotary color filter 140 that includes a plurality of filters that differ in spectral transmittance. The light source section 100 also includes a rotation driver section 150 that drives the rotary color filter 140, and a condenser lens 160 that focuses the light that has passed through the rotary color filter 140 on the incident end face of a light guide fiber 210. The light source aperture driver section 130 adjusts the intensity of light by opening and closing the light source aperture 120 based on a control signal output from a control section 302 included in the processor section 300.

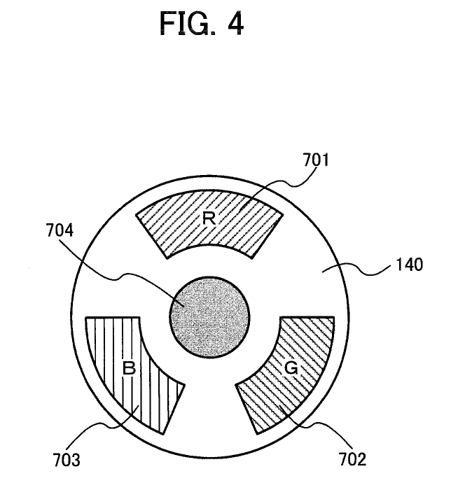

FIG. 4 illustrates a detailed configuration example of the rotary color filter 140. The rotary color filter 104 includes a red (R) color filter 701, a green (G) color filter 702, a blue (B) color filter 703, and a rotary motor 704. For example, the R color filter 701 allows light having a wavelength of 580 to 700 nm to pass through, the G color filter 702 allows light having a wavelength of 480 to 600 nm to pass through, and the B color filter 703 allows light having a wavelength of 400 to 500 nm to pass through. The rotation driver section 150 rotates the rotary color filter 140 at a given rotational speed in synchronization with the imaging period of an image sensor 260 based on the control signal output from the control section 302. For example, when the rotary color filter 140 is rotated at 20 revolutions per second, each color filter crosses the incident white light every 1/60th of a second. In this case, the image sensor 260 captures and transfers image signals every 1/60th of a second. The image sensor 260 is a monochrome single-chip image sensor, for example. The image sensor 260 is implemented by a CCD image sensor or a CMOS image sensor, for example. Specifically, the endoscope apparatus according to the first embodiment frame-sequentially captures an R image, a G image, and a B image every 1/60th of a second.

The imaging section 200 is formed to be elongated and flexible so that the imaging section 200 can be inserted into a body cavity, for example. The imaging section 200 includes the light guide fiber 210 that guides the light focused by the light source section 100, and an illumination lens 220 that diffuses the light guided by the light guide fiber 210 to illuminate the observation target. The imaging section 200 further includes an objective lens 230 that focuses the reflected light from the observation target, a focus lens 240 that adjusts the focal distance, a lens driver section 250 that moves the position of the focus lens 240, and the image sensor 260 that detects the focused reflected light. The lens driver section 250 is a voice coil motor (VCM), for example. The lens driver section 250 is connected to the focus lens 240. The lens driver section 250 adjusts the in-focus object plane position by switching the position of the focus lens 240 between consecutive positions.

The imaging section 200 is provided with a switch 270 that allows the user to issue an enhancement process ON/OFF instruction. When the user has operated the switch 270, an enhancement process ON/OFF instruction signal is output from the switch 270 to the control section 302.

The imaging section 200 includes a memory 211 that stores information about the imaging section 200. The memory 211 stores a scope ID that represents the intended usage of the imaging section 200, information about the optical properties of the imaging section 200, information about the functions of the imaging section 200, and the like. The scope ID is an ID that corresponds to a scope for a lower gastrointestinal tract (large intestine), a scope for an upper gastrointestinal tract (gullet and stomach), or the like. The information about the optical properties of the imaging section 200 is information about the magnification (angle of view) of the optical system, for example. The information about the functions of the imaging section 200 is information about the execution state of each function (e.g., water supply) of the scope, for example.

The processor section 300 (control device) controls each section of the endoscope apparatus, and performs image processing. The processor section 300 includes the control section 302 and an image processing section 301. The control section 302 is bidirectionally connected to each section of the endoscope apparatus, and controls each section of the endoscope apparatus. For example, the control section 302 changes the position of the focus lens 240 by transmitting the control signal to the lens driver section 250. The image processing section 301 performs a process that determines a mucous membrane area from the captured image, and performs an enhancement process on the determined mucous membrane area, for example. The details of the image processing section 301 are described later.

The display section 400 displays the endoscopic image transmitted from the processor section 300. The display section 400 is an image display device (e.g., endoscope monitor) that can display a moving image (movie), for example.

The external I/F section 500 is an interface that allows the user to input information and the like to the endoscope apparatus. The external I/F section 500 includes a power switch (power ON/OFF switch), a mode (e.g., imaging mode) switch button, an AF button (i.e., a button for starting an autofocus operation that automatically brings the object into focus), and the like.

### 2.2. Image Processing Section

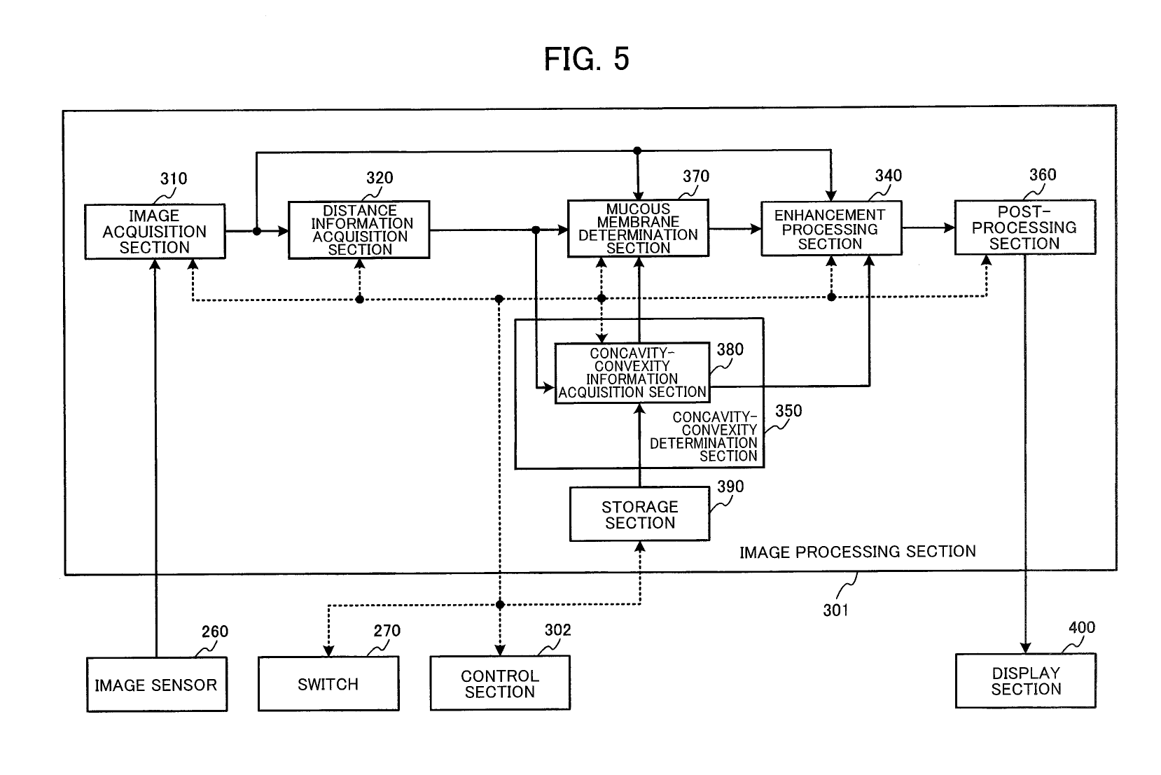

FIG. 5 illustrates a configuration example of the image processing section 301 according to the first embodiment. The image processing section 301 includes an image acquisition section 310, a distance information acquisition section 320, a mucous membrane determination section 370, an enhancement processing section 340, a post-processing section 360, a concavity-convexity determination section 350, and a storage section 390. The concavity-convexity determination section 350 includes a concavity-convexity information acquisition section 380.

The image acquisition section 310 is connected to the distance information acquisition section 320, the mucous membrane determination section 370, and the enhancement processing section 340. The distance information acquisition section 320 is connected to the mucous membrane determination section 370 and the concavity-convexity information acquisition section 380. The mucous membrane determination section 370 is connected to the enhancement processing section 340. The enhancement processing section 340 is connected to the post-processing section 360. The post-processing section 360 is connected to the display section 400. The concavity-convexity information acquisition section 380 is connected to the mucous membrane determination section 370 and the enhancement processing section 340. The storage section 390 is connected to the concavity-convexity information acquisition section 380. The control section 302 is bidirectionally connected to each section of the image processing section 301, and controls each section of the image processing section 301. For example, the control section 302 synchronizes the image acquisition section 310, the post-processing section 360, and the light source aperture driver section 130. The control section 302 transmits the enhancement process ON/OFF instruction signal from the switch 270 (or the external I/F section 500) to the enhancement processing section 340.

The image acquisition section 310 converts analog image signals transmitted from the image sensor 260 into digital image signals by performing an A/D conversion process. The image acquisition section 310 performs an OB clamp process, a gain control process, and a WB correction process on the digital image signals using an OB clamp value, a gain correction value, and a WB coefficient stored in the control section 302. The image acquisition section 310 performs a color image generating process on an R image, a G image, and a B image that have been captured frame-sequentially to acquire a color image that has RGB pixel values on a pixel basis. The image acquisition section 310 transmits the color image to the distance information acquisition section 320, the mucous membrane determination section 370, and the enhancement processing section 340 as an endoscopic image (captured image). Note that the A/D conversion process may be performed in the preceding stage (e.g., the imaging section 200) of the image processing section 301.

The distance information acquisition section 320 acquires distance information about the distance to the object based on the endoscopic image, and transmits the distance information to the mucous membrane determination section 370 and the concavity-convexity information acquisition section 380. For example, the distance information acquisition section 320 detects the distance to the object by calculating a defocus parameter from the endoscopic image. When the imaging section 200 includes an optical system that captures a stereo image, the distance information acquisition section 320 may detect the distance to the object by performing a stereo matching process on the stereo image. When the imaging section 200 includes a Time-of-Flight (TOF) sensor, the distance information acquisition section 320 may detect the distance to the object based on the sensor output. The details of the distance information acquisition section 320 are described later.

Note that the distance information represents a distance map that includes the distance information corresponding to each pixel of the endoscopic image, for example. The distance information includes information that represents the rough structure of the object, and information that represents concavities-convexities that are relatively smaller than the rough structure. The information that represents the rough structure corresponds to the rough undulations of the lumen structure and the mucous membrane of the internal organ, for example. The information that represents the rough structure is a low-frequency component of the distance information, for example. The information that represents the concavities-convexities corresponds to the concavities-convexities on the surface of the mucous membrane or a lesion, for example. The information that represents the concavities-convexities is a high-frequency component of the distance information, for example.

The concavity-convexity information acquisition section 380 extracts extracted concavity-convexity information that represents a concavity-convexity part of the surface of tissue from the distance information based on known characteristic information stored in the storage section 390. Specifically, the concavity-convexity information acquisition section 380 acquires the size (i.e., dimensional information such as width, height, or depth) of the extraction target concavity-convexity part as the known characteristic information, and extracts a concavity-convexity part that has the desired dimensional characteristics represented by the known characteristic information. The details of the concavity-convexity information acquisition section 380 are described later.

The mucous membrane determination section 370 determines the enhancement target mucous membrane area (e.g., an area of tissue where a lesion may be present) from the endoscopic image. For example, the mucous membrane determination section 370 determines an area that agrees with the color characteristics of a mucous membrane to be a mucous membrane area based on the endoscopic image (described later). Alternatively, the mucous membrane determination section 370 determines a concavity-convexity part among the concavity-convexity parts represented by the extracted concavity-convexity information that agrees with the characteristics of the enhancement target mucous membrane (e.g., concavity or groove) to be a mucous membrane area based on the extracted concavity-convexity information and the distance information. The mucous membrane determination section 370 determines whether or not each pixel corresponds to a mucous membrane, and outputs position information (coordinates) about a pixel that has been determined to correspond to a mucous membrane to the enhancement processing section 340. In this case, a set of pixels that have been determined to correspond to a mucous membrane corresponds to a mucous membrane area.

The enhancement processing section 340 performs the enhancement process on the determined mucous membrane area, and outputs the resulting endoscopic image to the post-processing section 360. When the mucous membrane determination section 370 determines a mucous membrane area based on color, the enhancement processing section 340 performs the enhancement process on the mucous membrane area based on the extracted concavity-convexity information. When the mucous membrane determination section 370 determines a mucous membrane area based on the extracted concavity-convexity information, the enhancement processing section 340 performs the enhancement process on the mucous membrane area without using the extracted concavity-convexity information. In either case, the enhancement process is performed based on the extracted concavity-convexity information. The enhancement process may be a process that enhances a concavity-convexity structure of a mucous membrane (e.g., a high-frequency component of an image), or may be a process that enhances a given color component corresponding to concavities-convexities of a mucous membrane. When the enhancement process enhances a color component, dye spraying may be simulated by thickening a given color component of a concavity as compared with a convexity, for example.

The post-processing section 360 performs a grayscale transformation process, a color process, and a contour enhancement process on the endoscopic image transmitted from the enhancement processing section 340 using a grayscale transformation coefficient, a color conversion coefficient, and a contour enhancement coefficient stored in the control section 302. The post-processing section 360 transmits the resulting endoscopic image to the display section 400.

### 2.3. Mucous Membrane Determination Process

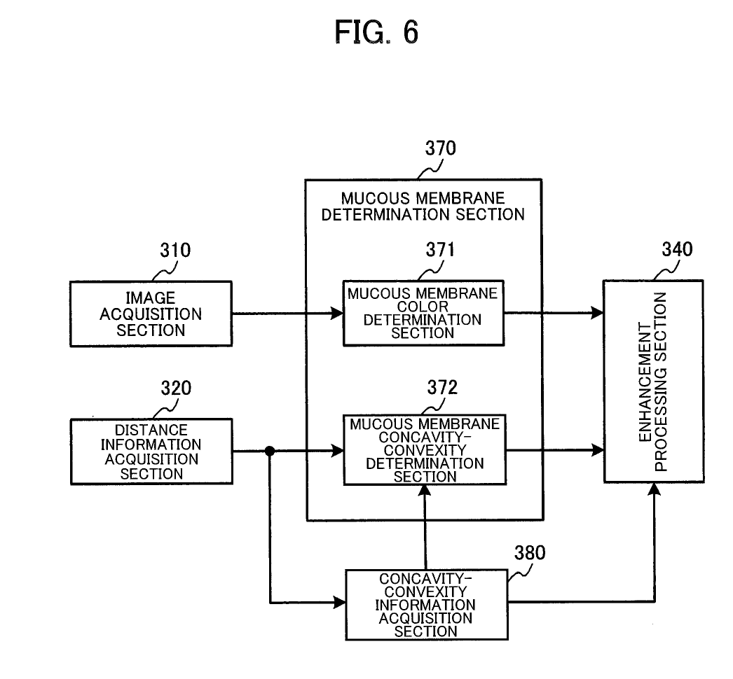

FIG. 6 illustrates a detailed configuration example of the mucous membrane determination section 370. The mucous membrane determination section 370 includes a mucous membrane color determination section 371 and a mucous membrane concavity-convexity determination section 372. In the first embodiment, at least one of the mucous membrane color determination section 371 and the mucous membrane concavity-convexity determination section 372 determines a mucous membrane area.

The mucous membrane color determination section 371 receives the endoscopic image transmitted from the image acquisition section 310. The mucous membrane color determination section 371 compares the hue value of each pixel of the endoscopic image with the hue value range of a mucous membrane, and determines whether or not each pixel corresponds to a mucous membrane. For example, the mucous membrane color determination section 371 determines a pixel for which the hue value H satisfies the following expression (1) to be a pixel that corresponds to a mucous membrane (hereinafter referred to as “mucous membrane pixel”).

10°<H≦30° (1)

The hue value H is calculated from the RGB pixel values using the following expression (2). The range of the hue value H is 0 to 360°. Note that max(R, G, B) in the expression (2) is the maximum value among the R pixel value, the G pixel value, and the B pixel value, and min(R, G, B) in the expression (2) is the minimum value among the R pixel value, the G pixel value, and the B pixel value. When the hue value H calculated using the expression (2) is a negative value, 360° is added to the hue value H.

$$H = \{ \begin{matrix}\frac{G - B}{max ( R , G , B ) - min ( R , G , B )} \times 60 & ( max ( R , G , B ) = R ) \\ \frac{B - R}{max ( R , G , B ) - min ( R , G , B )} \times 60 + 120 & ( max ( R , G , B ) = G ) \\ \frac{R - G}{max ( R , G , B ) - min ( R , G , B )} \times 60 + 240 & ( max ( R , G , B ) = B )\end{matrix} \qquad ( 2 )$$

It is possible to perform the enhancement process on only an area that is determined to be a mucous membrane from the color characteristics by thus determining a mucous membrane based on the color of each pixel. Since an object that need not be enhanced is not enhanced, it is possible to implement an enhancement process that is appropriate for a medical examination.

The mucous membrane concavity-convexity determination section 372 receives the distance information transmitted from the distance information acquisition section 320, and receives the extracted concavity-convexity information transmitted from the concavity-convexity information acquisition section 380. The mucous membrane concavity-convexity determination section 372 determines whether or not each pixel corresponds to a mucous membrane based on the distance information and the extracted concavity-convexity information. Specifically, the mucous membrane concavity-convexity determination section 372 detects a groove (e.g., a concavity having a width equal to or less than 1000 μm and a depth equal to or less than 100 μm) formed in the surface of tissue based on the extracted concavity-convexity information. The mucous membrane concavity-convexity determination section 372 determines a pixel that has been detected as a groove formed in the surface of tissue, and a pixel that satisfies the following expressions (3) and (4), to be the mucous membrane pixel. Note that the groove detection method is described later.

|D(x,y)−D(p,q)|<T_{neighbor} (3)

(p,q)∈R_{groove} (4)

The expression (4) represents a pixel situated at coordinates (p, q) is a pixel that has been detected as a groove formed in the surface of tissue. D(x, y) in the expression (3) is the distance to the object at a pixel situated at coordinates (x, y), and D(p, q) in the expression (3) is the distance to the object at a pixel situated at coordinates (p, q). These distances are the distance information acquired by the distance information acquisition section 320. T_{neighbor} is a threshold value for the difference in distance between pixels.

For example, the distance information acquisition section 320 acquires a distance map as the distance information. The term “distance map” used herein refers to a map in which the distance (depth) to the object in the Z-axis direction (i.e., the direction of the optical axis of the imaging section 200) is specified corresponding to each point (e.g., each pixel) in the XY plane, for example. For example, when the pixels of the endoscopic image and the pixels of the distance map have a one-to-one relationship, the distance D(x, y) at coordinates (x, y) of the endoscopic image is the value at coordinates (x, y) of the distance map.

It is possible to perform the enhancement process on only a groove formed in the surface of tissue and an area situated in the vicinity of the groove by thus determining a groove formed in the surface of tissue and an area situated in the vicinity of the groove to be a mucous membrane based on the distance information and the extracted concavity-convexity information. Since an object that need not be enhanced is not enhanced, it is possible to implement an enhancement process that is appropriate for a medical examination.

According to the first embodiment, a mucous membrane (i.e., enhance target) is determined from the endoscopic image based on the endoscopic image and the distance information, and the concavity-convexity information about the surface of the object is enhanced with respect to the determined mucous membrane based on the distance information. Since the enhancement process can be performed on an area for which the enhancement process is necessary, it is possible to improve the capability to discriminate between an area for which the enhancement process is necessary, and an area for which the enhancement process is unnecessary, and suppress as much as possible a situation in which the user gets tired when observing the image subjected to the enhancement process.

According to the first embodiment, the mucous membrane determination section 370 determines an area for which the feature quantity based on the pixel value of the captured image satisfies a given condition that corresponds to a mucous membrane, to be a mucous membrane area. More specifically, the mucous membrane determination section 370 determines an area for which color information (e.g., hue value) (i.e., feature quantity) satisfies a given condition (e.g., hue value range) relating to the color of a mucous membrane, to be a mucous membrane area.

This makes it possible to determine an object that should be enhanced based on the feature quantity of the image. Specifically, it is possible to determine an object that should be enhanced by setting the feature of a mucous membrane as a feature quantity condition, and detecting an area that satisfies the condition. For example, it is possible to determine an area that satisfies the condition (color condition) to be an object that should be enhanced by setting the color specific to a mucous membrane as a given condition.

The mucous membrane determination section 370 determines an area for which the extracted concavity-convexity information agrees with the concavity-convexity characteristics represented by the known characteristic information to be a mucous membrane area. More specifically, the mucous membrane determination section 370 acquires the dimensional information that represents at least one of the width and the depth of a concavity (groove) of the object as the known characteristic information, and extracts a concavity among the concavity-convexity parts included in the extracted concavity-convexity information that agrees with the characteristics specified by the dimensional information. The mucous membrane determination section 370 determines a concavity area within the captured image that corresponds to the extracted concavity, and an area situated in the vicinity of the concavity area, to be a mucous membrane area.

This makes it possible to determine an object that should be enhanced based on the concavity-convexity shape of the object. Specifically, it is possible to determine an object that should be enhanced by setting the feature of a mucous membrane as a concavity-convexity shape condition, and detecting an area that satisfies the condition. It is possible to perform the enhancement process on a concavity area by determining a concavity area to be a mucous membrane area. Since a concavity formed in the surface of tissue tends to be deeply stained by dye spraying (described later), it is possible to simulate dye spraying by image processing by enhancing a concavity.

The term “concavity-convexity characteristics” used herein refers to the characteristics of a concavity-convexity part specified (represented) by the concavity-convexity characteristic information. The term “concavity-convexity characteristic information” used herein refers to information that specifies (represents) the characteristics of a concavity-convexity part of the object that is to be extracted from the distance information. Specifically, the concavity-convexity characteristic information includes at least one of information that represents the characteristics of the non-extraction target concavity-convexity part (concavities-convexities) among the concavity-convexity parts (concavities-convexities) included in the distance information, and information that represents the characteristics of the extraction target concavity-convexity part (concavities-convexities) among the concavity-convexity parts (concavities-convexities) included in the distance information.

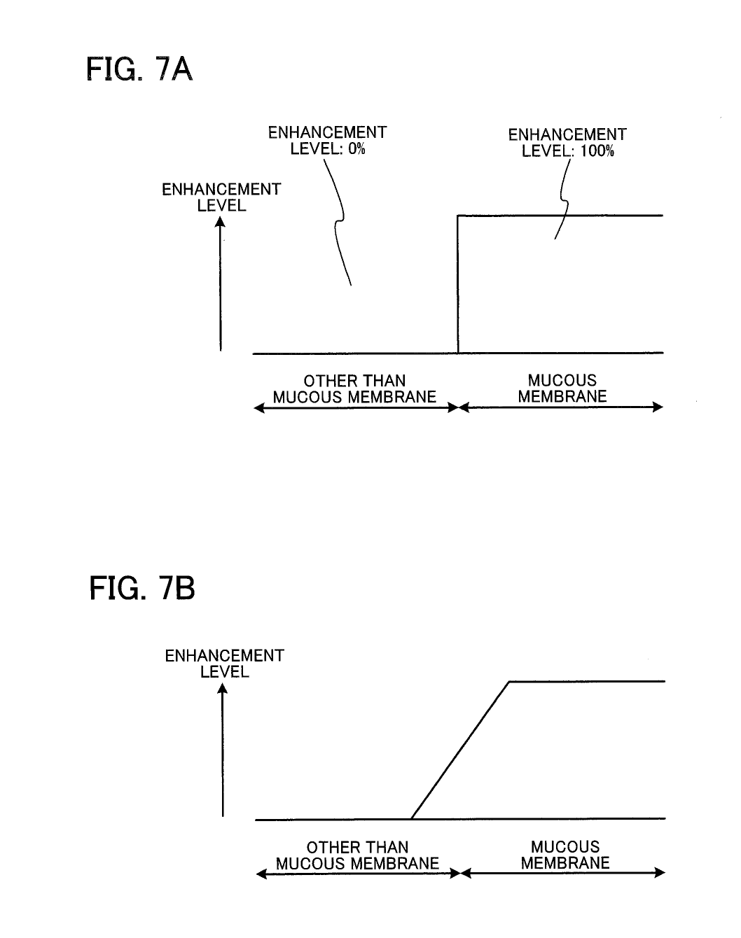

According to the first embodiment, the enhancement process is performed in a binary way (i.e., the enhancement process is performed (ON) on a mucous membrane area, and is not performed (OFF) on an area other than a mucous membrane area) (see FIG. 7A). Note that the configuration is not limited thereto. The enhancement processing section 340 may perform the enhancement process using the enhancement level that continuously changes at the boundary between a mucous membrane area and an area other than a mucous membrane area (see FIG. 7B). Specifically, a low-pass filtering process is performed on the enhancement level at the boundary between a mucous membrane area and an area other than a mucous membrane area so that the enhancement level continuously changes (e.g., 0 to 100%).

In this case, since the ON/OFF boundary of the enhancement process is not clearly observed, the user can observe the endoscopic image without problems as compared with the case where the enhancement level is discontinuously changed at the boundary. This makes it possible to suppress a situation in which an unnatural endoscopic image is observed.

### 2.4. First Concavity-Convexity Information Acquisition Process

FIG. 8 illustrates a detailed configuration example of the concavity-convexity information acquisition section 380. The concavity-convexity information acquisition section 380 includes a known characteristic information acquisition section 381, an extraction section 383, and an extracted concavity-convexity information output section 385.

A method that sets an extraction process parameter based on the known characteristic information, and extracts the extracted concavity-convexity information from the distance information using an extraction process that utilizes the extraction process parameter, is described below. Specifically, a concavity-convexity part having the desired dimensional characteristics (i.e., a concavity-convexity part having a width within the desired range in a narrow sense) is extracted as the extracted concavity-convexity information using the known characteristic information. Since the three-dimensional structure of the object is reflected in the distance information, the distance information includes information about the desired concavity-convexity part, and information about a global structure that is larger than the desired concavity-convexity part, and corresponds to a fold structure, and a wall surface structure of a lumen. Specifically, the extracted concavity-convexity information acquisition process according to the first embodiment may be referred to as a process that excludes information about a fold structure and a lumen structure from the distance information.

Note that the extracted concavity-convexity information acquisition process is not limited thereto. For example, the extracted concavity-convexity information acquisition process may not utilize the known characteristic information. When the extracted concavity-convexity information acquisition process utilizes the known characteristic information, various types of information may be used as the known characteristic information. For example, the extraction process may exclude information about a lumen structure from the distance information, but allow information about a fold structure to remain. In such a case, it is also possible to determine the desired object to be a mucous membrane since the known characteristic information (e.g., dimensional information about a concavity) is used during the mucous membrane concavity-convexity determination process.

The known characteristic information acquisition section 381 acquires the known characteristic information from the storage section 390. Specifically, the known characteristic information acquisition section 381 acquires the size (i.e., dimensional information (e.g., width, height, or depth)) of the extraction target concavity-convexity part of tissue due to a lesion, the size (i.e., dimensional information (e.g., width, height, or depth)) of the lumen and the folds specific to the observation target part based on observation target part information, and the like as the known characteristic information. Note that the observation target part information is information that represents the observation target part that is determined based on scope ID information, for example. The observation target part information may also be included in the known characteristic information. For example, when the scope is an upper gastrointestinal scope, the observation target part is the gullet, the stomach, or the duodenum. When the scope is a lower gastrointestinal scope, the observation target part is the large intestine. Since the dimensional information about the extraction target concavity-convexity part and the dimensional information about the lumen and the folds specific to the observation target part differ corresponding to each part, the known characteristic information acquisition section 381 outputs information about a typical size of a lumen and folds acquired based on the observation target part information to the extraction section 383, for example. Note that the observation target part information need not necessarily be determined based on the scope ID information. For example, the user may select the observation target part information using a switch provided to the external I/F section 500.

The extraction section 383 determines the extraction process parameter based on the known characteristic information, and performs the extracted concavity-convexity information extraction process based on the determined extraction process parameter.

The extraction section 383 performs a low-pass filtering process using a given size (N×N pixels) on the input distance information to extract rough distance information. The extraction section 383 adaptively determines the extraction process parameter based on the extracted rough distance information. The details of the extraction process parameter are described later. The extraction process parameter may be the morphological kernel size (i.e., the size of a structural element) that is adapted to the distance information at the plane position orthogonal to the distance information of the distance map, the low-pass characteristics of a low-pass filter adapted to the distance information at the plane position, or the high-pass characteristics of a high-pass filter adapted to the plane position, for example. Specifically, the extraction process parameter is change information that changes an adaptive nonlinear or linear low-pass filter or high-pass filter corresponding to the distance information. Note that the low-pass filtering process is performed to suppress a decrease in the accuracy of the extraction process that may occur when the extraction process parameter changes frequently or significantly corresponding to the position within the image. The low-pass filtering process may not be performed when a decrease in the accuracy of the extraction process is negligible.

The extraction section 383 performs the extraction process based on the determined extraction process parameter to extract only the concavity-convexity parts of the object having the desired size. The extracted concavity-convexity information output section 385 outputs the extracted concavity-convexity parts to the mucous membrane determination section 370 and the enhancement processing section 340 as the extracted concavity-convexity information (concavity-convexity image) having the same size as that of the captured image (i.e., the image subjected to the enhancement process).

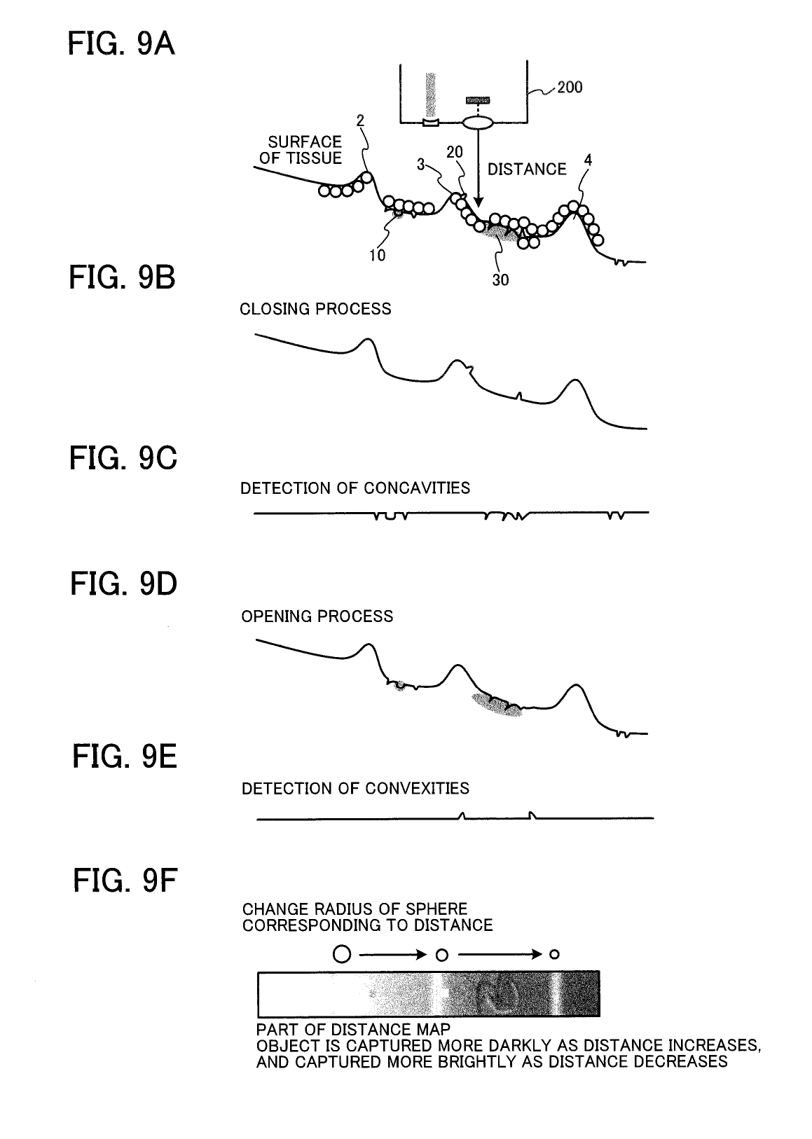

The details of the extraction process parameter determination process performed by the extraction section 383 are described below with reference to FIGS. 9A to 9F. In FIGS. 9A to 9F, the extraction process parameter is the diameter of a structural element (sphere) that is used for an opening process and a closing process (morphological process). FIG. 9A is a view schematically illustrating the surface of the object (tissue) and the vertical cross section of the imaging section 200. The folds 2, 3, and 4 present on the surface of the tissue are gastric folds, for example. The early lesions 10, 20, and 30 are present on the surface of the tissue.

The extraction process parameter determination process performed by the extraction section 383 is intended to determine the extraction process parameter for extracting only the early lesions 10, 20, and 30 from the surface of tissue without extracting the folds 2, 3, and 4.

In order to determine such an extraction process parameter, it is necessary to use the size (i.e., dimensional information (e.g., width, height, or depth)) of the extraction target concavity-convexity part of tissue due to a lesion, and the size (i.e., dimensional information (e.g., width, height, or depth)) of the lumen and the folds specific to the observation target part based on the observation target part information (that are stored in the storage section 390).

It is possible to extract only the desired concavity-convexity part by determining the diameter of the sphere (with which the surface of tissue is traced during the opening process and the closing process) using the above information. The diameter of the sphere is set to be smaller than the size of the lumen and the folds specific to the observation target part based on the observation target part information, and larger than the size of the extraction target concavity-convexity part of tissue due to a lesion. It is desirable to set the diameter of the sphere to be equal to or smaller than half of the size of the folds, and equal to or larger than the size of the extraction target concavity-convexity part of tissue due to a lesion. FIGS. 9A to 9F illustrate an example in which a sphere that satisfies the above conditions is used for the opening process and the closing process.

FIG. 9B illustrates the surface of tissue after the closing process has been performed. As illustrated in FIG. 9B, information in which the concavities among the concavity-convexity parts having the extraction target dimensions are filled while maintaining a change in distance due to the wall surface of the tissue, and the structures such as the folds, is obtained by determining an appropriate extraction process parameter (i.e., the size of the structural element). Only the concavities formed in the surface of the tissue can be extracted (see FIG. 9C) by calculating the difference between information obtained by the closing process and the original surface of the tissue (see FIG. 9A).

FIG. 9D illustrates the surface of the tissue after the opening process has been performed. As illustrated in FIG. 9D, information in which the convexities among the concavity-convexity parts having the extraction target dimensions are removed is obtained by the opening process. Only the convexities on the surface of the tissue can be extracted (see FIG. 9E) by calculating the difference between information obtained by the opening process and the original surface of the tissue.

The opening process and the closing process may be performed on the surface of the tissue using a sphere having an identical size. However, since the captured image is characterized in that the area of the image formed on the image sensor decreases as the distance represented by the distance information increases, the diameter of the sphere may be increased when the distance represented by the distance information is short, and may be decreased when the distance represented by the distance information is long, in order to extract a concavity-convexity part having the desired size.

As illustrated in FIG. 9F, the diameter of the sphere is changed with respect to the average distance information when performing the opening process and the closing process on the distance map. Specifically, it is necessary to correct the actual size of the surface of the tissue using the optical magnification to agree with the pixel pitch of the image formed on the image sensor in order to extract the desired concavity-convexity part with respect to the distance map. Therefore, it is desirable that the extraction section 383 acquire the optical magnification or the like of the imaging section 200 that is determined based on the scope ID information.

Specifically, the process that determines the size of the structural element (extraction process parameter) is performed so that the exclusion target shape (e.g., folds) is not deformed (i.e., the sphere moves to follow the exclusion target shape) when the process using the structural element is performed on the exclusion target shape (when the sphere is moved on the surface in FIG. 9A). The size of the structural element may be determined so that the extraction target concavity-convexity part (extracted concavity-convexity information) is removed (i.e., the sphere does not enter the concavity or the convexity) when the process using the structural element is performed on the extraction target concavity-convexity part. Since the morphological process is a well-known process, detailed description thereof is omitted.

According to the first embodiment, the concavity-convexity information acquisition section 380 determines the extraction process parameter based on the known characteristic information, and extracts a concavity-convexity part of the object as the extracted concavity-convexity information based on the determined extraction process parameter.

This makes it possible to perform the extracted concavity-convexity information extraction process (e.g., separation process) using the extraction process parameter determined based on the known characteristic information. The extraction process may be performed using the morphological process (see above), a filtering process (described later), or the like. In order to accurately extract the extracted concavity-convexity information, it is necessary to perform a control process that extracts information about the desired concavity-convexity part from the information about various structures included in the distance information while excluding other structures (e.g., the structures specific to tissue, such as folds). In the first embodiment, such a control process is implemented by setting the extraction process parameter based on the known characteristic information.

The captured image may be an in vivo image that is obtained by capturing the inside of a living body, and the known characteristic information acquisition section 381 may acquire part information and concavity-convexity characteristic information as the known characteristic information, the part information being information that represents a part of the living body to which the object corresponds, and the concavity-convexity characteristic information being information about a concavity-convexity part of the living body. The concavity-convexity information acquisition section 380 may determine the extraction operation parameter based on the part information and the concavity-convexity characteristic information.

This makes it possible to acquire the part information about a part (object) within an in vivo image as the known characteristic information when applying the method according to the first embodiment to an in vivo image (e.g., when applying the image processing device according to the first embodiment to a medical endoscope apparatus). When applying the method according to the first embodiment to an in vivo image, it is considered that a concavity-convexity structure that is useful for detecting an early lesion or the like is extracted as the extracted concavity-convexity information. However, the characteristics (e.g., dimensional information) of a concavity-convexity part specific to an early lesion may differ corresponding to each part. Moreover, the exclusion target structure (e.g., folds) necessarily differs corresponding to each part. Therefore, it is necessary to perform an appropriate process corresponding to each part when applying the method according to the first embodiment to an in vivo image. In the first embodiment, such a process is performed based on the part information.

The concavity-convexity information acquisition section 380 may determine the size of the structural element used for the opening process and the closing process as the extraction process parameter based on the known characteristic information, and perform the opening process and the closing process using the structural element having the determined size to extract a concavity-convexity part of the object as the extracted concavity-convexity information.

This makes it possible to extract the extracted concavity-convexity information based on the opening process and the closing process (morphological process in a broad sense). In this case, the extraction process parameter is the size of the structural element used for the opening process and the closing process. In the example illustrated in FIG. 9A, the structural element is a sphere, and the extraction process parameter is a parameter that represents the diameter of the sphere, for example.

### 2.5. Second Concavity-Convexity Information Acquisition Process

The extraction process according to the first embodiment is not limited to a morphological process. The extraction process may be implemented using a filtering process. For example, when using a low-pass filtering process, the characteristics of the low-pass filter are determined so that the extraction target concavity-convexity part of tissue due to a lesion can be smoothed, and the structure of the lumen and the folds specific to the observation target part can be maintained. Since the characteristics of the extraction target (i.e., concavity-convexity part) and the exclusion target (i.e., folds and lumen) can be determined from the known characteristic information, the spatial frequency characteristics are known, and the characteristics of the low-pass filter can be determined

The low-pass filter may be a known Gaussian filter or bilateral filter. The characteristics of the low-pass filter may be controlled using a parameter σ, and a σ map corresponding to each pixel of the distance map may be generated. When using a bilateral filter, the σ map may be generated using either or both of a luminance difference parameter σ and a distance parameter σ. A Gaussian filter is represented by the following expression (5), and a bilateral filter is represented by the following expression (6).

$$\begin{matrix}f ( x ) = \frac{1}{N} exp ( - \frac{{( x - x 0 )}^{2}}{2 {σ}^{2}} ) & ( 5 ) \\ f ( x ) = \frac{1}{N} exp ( - \frac{( x - x 0 )}{2 {σ}_{c}^{2}} ) \times exp ( - \frac{{( p ( x ) - p ( x 0 ) )}^{2}}{2 {σ}_{v}^{2}} ) & ( 6 )\end{matrix}$$

For example, a σ map subjected to a thinning process may be generated, and the desired low-pass filter may be applied to the distance map using the σ map.

The parameter σ that determines the characteristics of the low-pass filter is set to be larger than the value obtained by multiplying the pixel-to-pixel distance D1 of the distance map corresponding to the size of the extraction target concavity-convexity part by α (>1), and smaller than the value obtained by multiplying the pixel-to-pixel distance D2 of the distance map corresponding to the size of the lumen and the folds specific to the observation target part by β (<1). For example, the parameter σ may be calculated by σ=(α*D1+β*D2)/2*Rσ.

Steeper sharp-cut characteristics may be set as the characteristics of the low-pass filter. In this case, the filter characteristics are controlled using a cut-off frequency fc instead of the parameter σ. The cut-off frequency fc may be set so that a frequency F1 in the cycle D1 does not pass through, and a frequency F2 in the cycle D2 does pass through. For example, the cut-off frequency fc may be set to fc=(F1+F2)/2*Rf.

Note that Rσ is a function of the local average distance. The output value increases as the local average distance decreases, and decreases as the local average distance increases. Rf is a function that is designed so that the output value decreases as the local average distance decreases, and increases as the local average distance increases.

A concavity image can be output by extracting only a negative area obtained by subtracting the low-pass filtering results from the distance map that is not subjected to the low-pass filtering process. A convexity image can be output by extracting only a positive area obtained by subtracting the low-pass filtering results from the distance map that is not subjected to the low-pass filtering process.

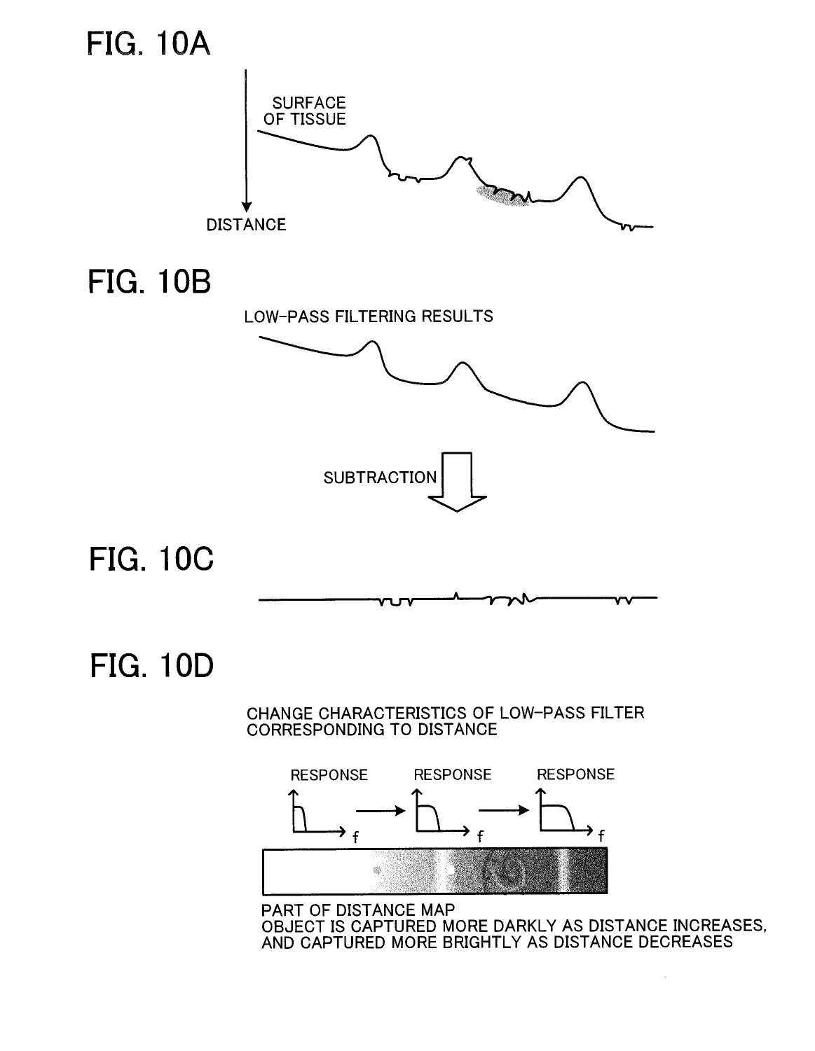

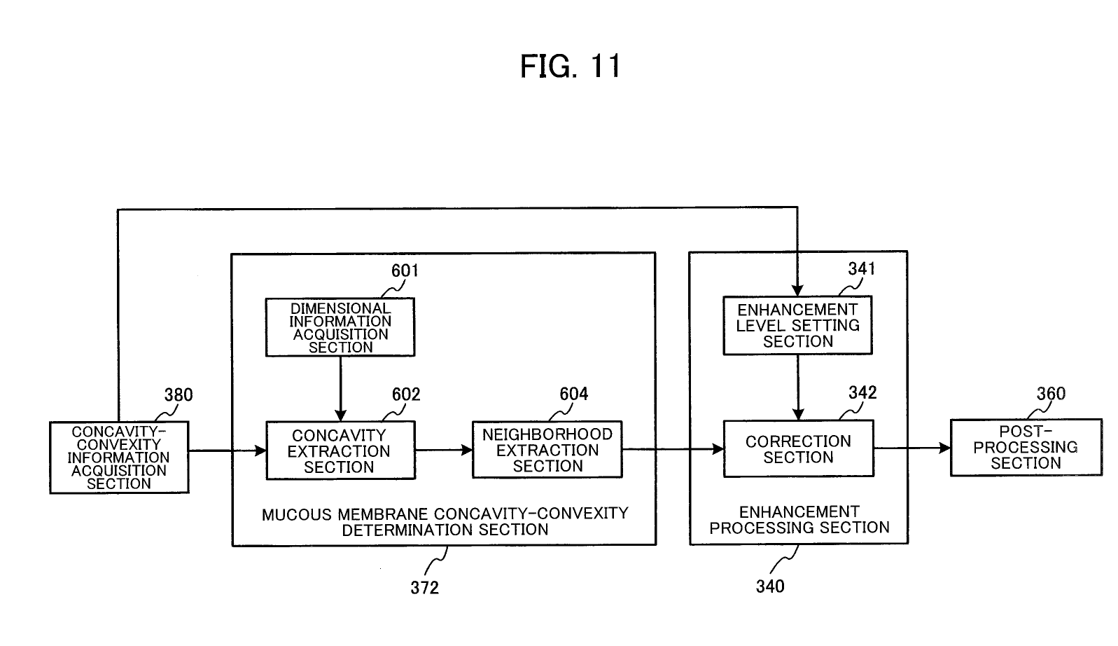

FIGS. 10A to 10D illustrate extraction of the desired concavity-convexity part due to a lesion using the low-pass filter. As illustrated in FIG. 10B, information in which the concavity-convexity parts having the extraction target dimensions are removed while maintaining the change in distance due to the wall surface of the tissue, and the structures such as the folds, is obtained by performing the filtering process using the low-pass filter on the distance map illustrated in FIG. 10A. Since the low-pass filtering results serve as a reference plane for extracting the desired concavity-convexity parts (see FIG. 10B) even if the opening process and the closing process described above are not performed, the concavity-convexity parts can be extracted (see FIG. 10C) by performing a subtraction process on the distance map (see FIG. 10A). When using the opening process and the closing process, the size of the structural element is adaptively changed corresponding to the rough distance information. When using the low-pass filtering process, it is desirable to change the characteristics of the low-pass filter corresponding to the rough distance information. FIG. 10D illustrates an example in which the characteristics of the low-pass filter are changed corresponding to the rough distance information.

A high-pass filtering process may be performed instead of the low-pass filtering process. In this case, the characteristics of the high-pass filter are determined so that the extraction target concavity-convexity part of tissue due to a lesion is maintained while removing the structure of the lumen and the folds specific to the observation target part.

The filter characteristics of the high-pass filter are controlled using a cut-off frequency fhc, for example. The cut-off frequency fhc may be set so that the frequency F1 in the cycle D1 passes through, and the frequency F2 in the cycle D2 does not pass through. For example, the cut-off frequency fhc may be set to fhc=(F1+F2)/2*Rf. Note that Rf is a function that is designed so that the output value decreases as the local average distance decreases, and increases as the local average distance increases.

The extraction target concavity-convexity part due to a lesion can be extracted directly by performing the high-pass filtering process. Specifically, the extracted concavity-convexity information is acquired directly (see FIG. 10C) without performing a difference calculation process.

According to the first embodiment, the concavity-convexity information acquisition section 380 determines the frequency characteristics of the filter used for the filtering process performed on the distance information as the extraction process parameter based on the known characteristic information, and perform the filtering process that utilizes the filter having the determined frequency characteristics to extract the concavity-convexity part of the object as the extracted concavity-convexity information.

This makes it possible to extract the extracted concavity-convexity information based on the filtering process. In this case, the extraction process parameter is the characteristics (i.e., spatial frequency characteristics in a narrow sense) of the filter used for the filtering process. Specifically, the parameter 6 and the cut-off frequency are determined based on the frequency that corresponds to the exclusion target (e.g., folds) and the frequency that corresponds to the concavity-convexity part (see above).

### 2.6. Mucous Membrane Concavity-Convexity Determination Process and Enhancement Process

The process (performed by the mucous membrane concavity-convexity determination section 372) that extracts a concavity (hereinafter may be referred to as “groove”) formed in the surface of tissue and an area situated in the vicinity of the concavity to be a mucous membrane, and the process (performed by the enhancement processing section 340) that enhances a mucous membrane area, are described in detail below. For example, the enhancement processing section 340 generates an image that simulates an image in which indigo carmine (that improves the contrast of minute concavity-convexity parts on the surface of tissue) is sprayed. Specifically, the enhancement processing section 340 multiplies the pixel values in a groove area and an area situated in the vicinity of the groove area by a gain that increases the degree of blueness. Note that the extracted concavity-convexity information transmitted from the concavity-convexity information acquisition section 380 corresponds to the endoscopic image input from the image acquisition section 310 on a pixel basis (on a one-to-one basis).

FIG. 11 illustrates a detailed configuration example of the mucous membrane concavity-convexity determination section 372. The mucous membrane concavity-convexity determination section 372 includes a dimensional information acquisition section 601, a concavity extraction section 602, and a neighborhood extraction section 604.

The dimensional information acquisition section 601 acquires the known characteristic information (particularly the dimensional information) from the storage section 390 or the like. The concavity extraction section 602 extracts the enhancement target concavity from the concavity-convexity parts included in (represented by) the extracted concavity-convexity information based on the known characteristic information. The neighborhood extraction section 604 extracts the surface of tissue situated within a given distance from the extracted concavity (i.e., situated in the vicinity of the extracted concavity).

When the mucous membrane concavity-convexity determination process has started, the mucous membrane concavity-convexity determination section 372 detects a groove formed in the surface of tissue from the extracted concavity-convexity information based on the known characteristic information. The known characteristic information represents the width and the depth of a groove formed in the surface of tissue. A minute groove formed in the surface of tissue normally has a width equal to or smaller than several thousand micrometers and a depth equal to or smaller than several hundred micrometers. The width and the depth of the groove formed in the surface of tissue are calculated from the extracted concavity-convexity information.

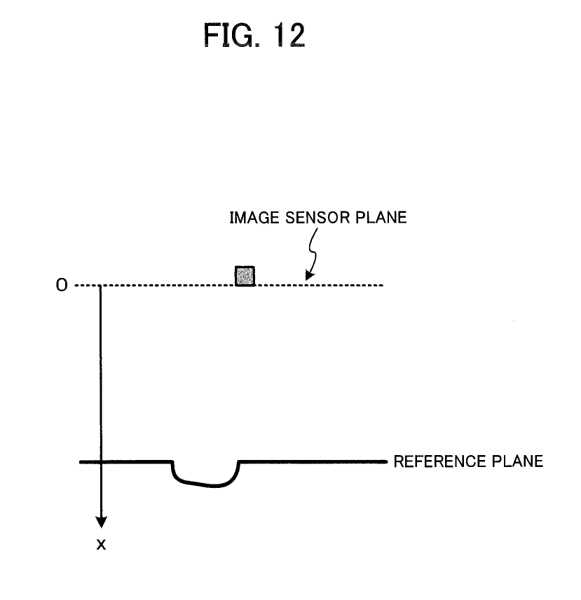

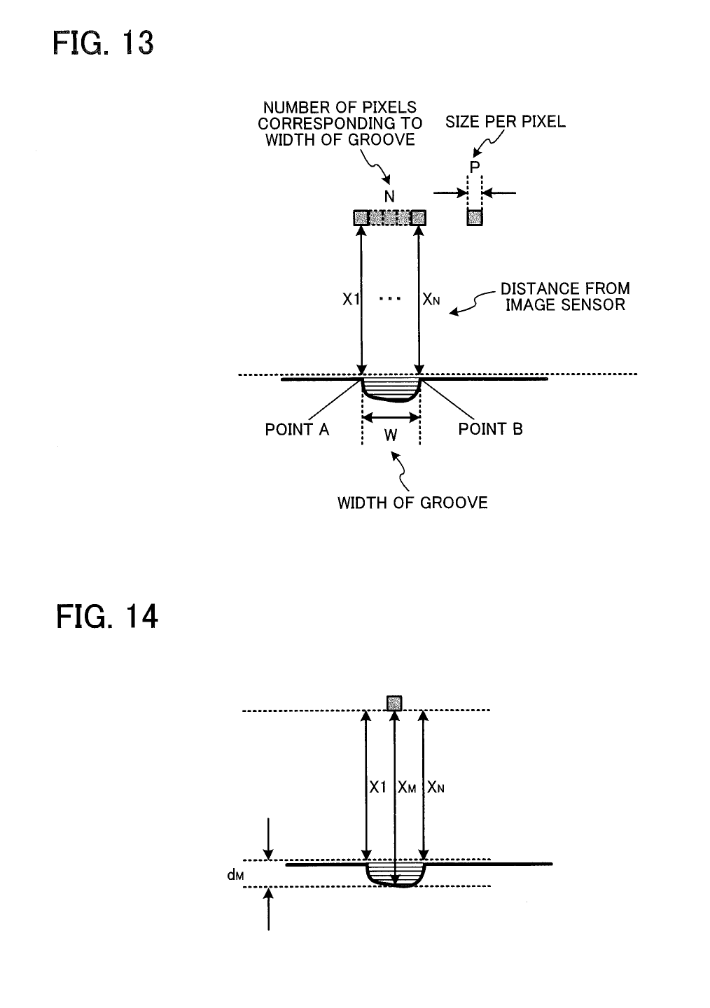

FIG. 12 illustrates one-dimensional extracted concavity-convexity information. The distance from the image sensor 260 to the surface of tissue increases in the depth direction provided that the position (imaging plane) of the image sensor 260 is 0. FIG. 13 illustrates a groove depth calculation method. Specifically, the ends of sequential points that are situated deeper than the reference plane and apart from the imaging plane at a distance equal to or larger than a given threshold value x1 (i.e., the points A and B illustrated in FIG. 13) are detected from the extracted concavity-convexity information. In the example illustrated in FIG. 13, the reference plane is situated at the distance x1 from the imaging plane. The number N of pixels that correspond to the points A and B and the points situated between the points A and B is calculated. The average value xave of the distances x1 to xN from the image sensor (at which the points A and B and the points situated between the points A and B are respectively situated) is calculated. The width w of the groove is calculated by the following expression (7). Note that p is the width per pixel of the image sensor 260, and K is the optical magnification that corresponds to the distance xave from the image sensor on a one-to-one basis.

w=N×p×K (7)

FIG. 14 illustrates a groove depth calculation method. The depth d of the groove is calculated by the following expression (8). Note that xM is the maximum value among the distances x1 to xN, and xmin is the distance x1 or xN, whichever is smaller.

d=xM−xmin1 (8)

The user may arbitrarily set the reference plane (i.e., the plane situated at the distance x1 from the image sensor) through the external I/F section 500. When the width and the depth of the groove thus calculated agree with the known characteristic information, the corresponding pixel positions of the endoscopic image are determined to be pixels that correspond to a groove area. For example, when the width of the groove is equal to or smaller than 3000 μm, and the depth of the groove is equal to or smaller than 500 μm, the corresponding pixels are determined to be pixels that correspond to a groove area. The user may set the threshold values (i.e., the width and the depth of a groove) through the external I/F section 500.

The neighborhood extraction section 604 detects neighborhood pixels that correspond to the surface of tissue and are situated within a given distance from the groove area in the depth direction (see FIG. 6). The pixels that correspond to the groove area and the pixels that correspond to the neighborhood area are output to the enhancement processing section 340 as mucous membrane pixels.

It is possible to determine the enhancement target object (i.e., the enhancement target pixels in a narrow sense) by performing the above process.

The enhancement process performed on the object is described below. The enhancement processing section 340 includes an enhancement level setting section 341 and a correction section 342. The enhancement processing section 340 multiplies the pixel values of the mucous membrane pixels by a gain coefficient. Specifically, the enhancement processing section 340 increases the signal value of the B signal of the attention pixel by multiplying the pixel value by the gain coefficient that is equal to or larger than 1, and decreases the signal values of the R signal and the G signal of the attention pixel by multiplying the pixel values by the gain coefficient that is equal to or smaller than 1. This makes it possible to obtain an image in which the degree of blueness of the groove (concavity) formed in the surface of tissue is increased (i.e., an image that simulates an image in which indigo carmine is sprayed).

The correction section 342 performs a correction process that improves the visibility of the enhancement target. The details thereof are described later. The correction section 342 may perform the correction process using the enhancement level that has been set by the enhancement level setting section 341.

The enhancement process ON/OFF instruction signal is input from the switch 270 or the external I/F section 500 through the control section 302. When the instruction signal instructs not to perform the enhancement process, the enhancement processing section 340 transmits the endoscopic image input from the image acquisition section 310 to the post-processing section 360 without performing the enhancement process. When the instruction signal instructs to perform the enhancement process, the enhancement processing section 340 performs the enhancement process.

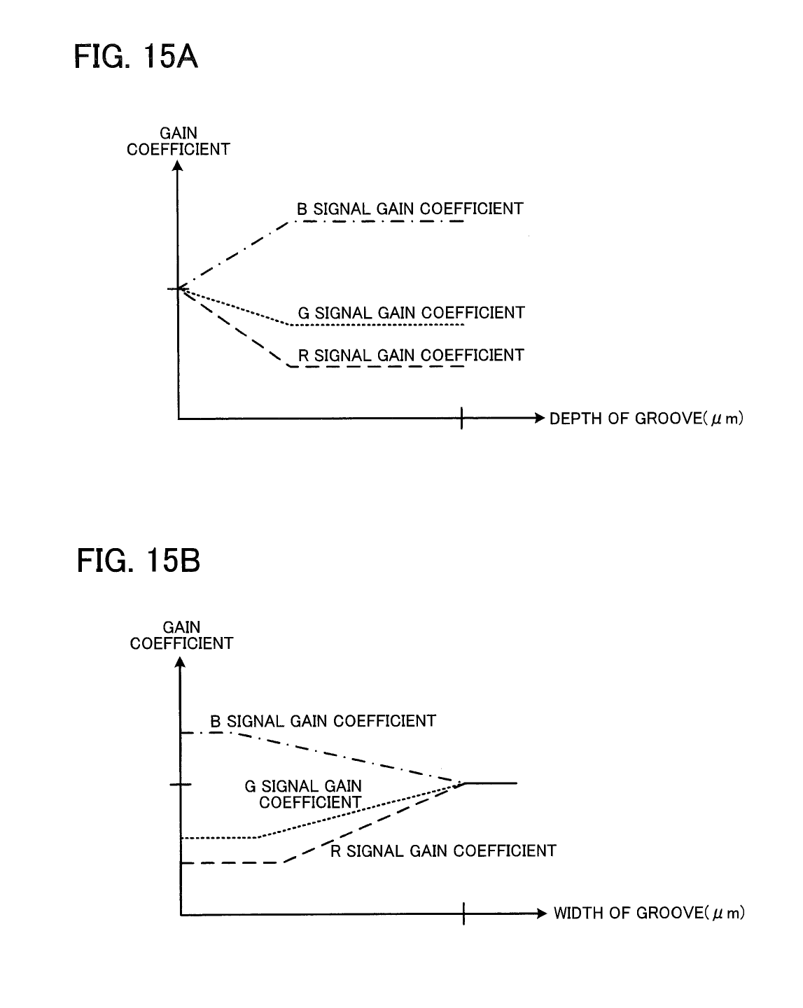

The enhancement processing section 340 may uniformly perform the enhancement process on the mucous membrane pixels. For example, the enhancement processing section 340 may perform the enhancement process on the mucous membrane pixels using an identical gain coefficient. Note that the enhancement processing section 340 may perform the enhancement process on the mucous membrane pixels in a different way. For example, the enhancement processing section 340 may perform the enhancement process on the mucous membrane pixels while changing the gain coefficient corresponding to the width and the depth of the groove. Specifically, the enhancement processing section 340 may multiply the pixel value by the gain coefficient so that the degree of blueness decreases as the depth of the groove decreases. This makes it possible to obtain an image that is closer to an image obtained by spraying a dye. FIG. 15A illustrates a gain coefficient setting example when multiplying the pixel value by the gain coefficient so that the degree of blueness decreases as the depth of the groove decreases. Alternatively, when it has been found that a fine structure is useful for finding a lesion, for example, the enhancement level may be increased as the width of the groove decreases (i.e., as the degree of fineness of the structure increases). FIG. 15B illustrates a gain coefficient setting example when increasing the enhancement level as the width of the groove decreases.

Although an example in which the enhancement process increases the degree of blueness has been described above, the configuration is not limited thereto. For example, the color to be applied to a groove may be changed corresponding to the depth of a groove. This makes it possible to visually observe the continuity of a groove as compared with the case where the same color is applied to each groove independently of the depth of the groove, and implement a highly accurate diagnosis.

Although an example has been described above in which the enhancement process increases the signal value of the B signal, and decreases the signal values of the R signal and the G signal by multiplying the pixel value by an appropriate gain coefficient, the configuration is not limited thereto. For example, the enhancement process may increase the signal value of the B signal and decrease the signal value of the R signal by multiplying the pixel value by an appropriate gain coefficient, while allowing the signal value of the G signal to remain unchanged. In this case, since the signal values of the B signal and the G signal remain although the degree of blueness of the concavity is increased, the structure within the concavity is displayed in cyan.

The enhancement process may be performed on the entire image instead of performing the enhancement process only on the mucous membrane pixels. In this case, the enhancement processing section 340 performs a process that improves visibility (i.e., a process that increases the gain coefficient) on an area that has been determined to be a mucous membrane, and performs a process that decreases the gain coefficient, sets the gain coefficient to 1 (original color), or changes the color to a specific color (e.g., a process that improves the visibility of the enhancement target by changing the color to the complementary color of the target color of the enhancement target) on the remaining area, for example. Specifically, the enhancement process according to the first embodiment is not limited to a process that generates an image that simulates an image obtained by spraying indigo carmine, but can be implemented by various processes that improve the visibility of the attention target.

### 2.7. Distance Information Acquisition Process

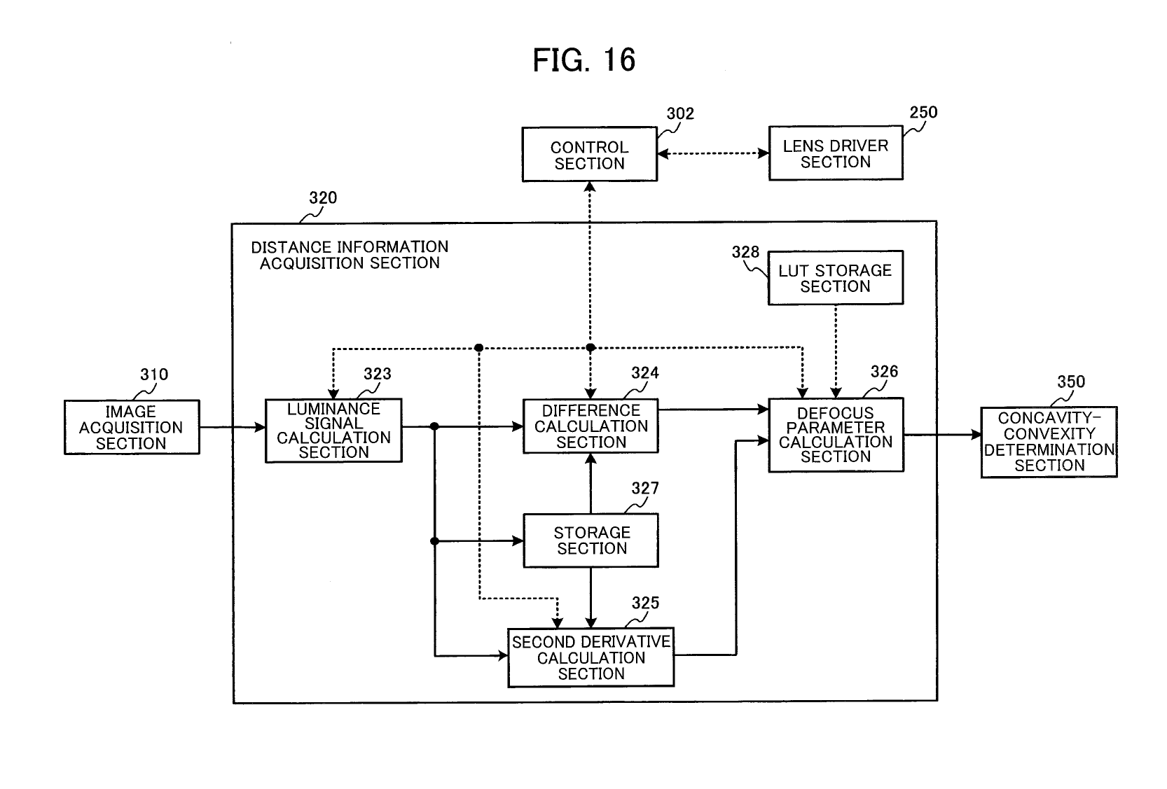

FIG. 16 illustrates a detailed configuration example of the distance information acquisition section 320. The distance information acquisition section 320 includes a luminance signal calculation section 323, a difference calculation section 324, a second derivative calculation section 325, a defocus parameter calculation section 326, a storage section 327, and an LUT storage section 328.

The luminance signal calculation section 323 calculates a luminance signal Y from the captured image output from the image acquisition section 310 using the following expression (9) under control of the control section 302.

Y=0.299×R+0.587×G+0.114×B (9)

The calculated luminance signal Y is transmitted to the difference calculation section 324, the second derivative calculation section 325, and the storage section 327. The difference calculation section 324 calculates the difference between the luminance signals Y from a plurality of images necessary for calculating the defocus parameter. The second derivative calculation section 325 calculates the second derivative of the luminance signals Y of the image, and calculates the average value of the second derivatives obtained from a plurality of luminance signals Y that differ in the degree of defocus. The defocus parameter calculation section 326 calculates the defocus parameter by dividing the difference between the luminance signals Y calculated by the difference calculation section 324 by the average value of the second derivatives calculated by the second derivative calculation section 325.

The storage section 327 stores the luminance signals Y of the first captured image, and the second derivative results thereof. Therefore, the distance information acquisition section 320 can place the focus lens at different positions through the control section 302, and acquire a plurality of luminance signals Y at different times. The LUT storage section 328 stores the relationship between the defocus parameter and the object distance in the form of a look-up table (LUT).

The control section 302 is bidirectionally connected to the luminance signal calculation section 323, the difference calculation section 324, the second derivative calculation section 325, and the defocus parameter calculation section 326, and controls the luminance signal calculation section 323, the difference calculation section 324, the second derivative calculation section 325, and the defocus parameter calculation section 326.

An object distance calculation method is described below. The control section 302 calculates the optimum focus lens position using a known contrast detection method, a known phase detection method, or the like based on the imaging mode set in advance using the external I/F section 500. The lens driver section 250 drives the focus lens 240 to the calculated focus lens position based on the signal output from the control section 302. The image sensor 260 acquires the first image of the object at the focus lens position to which the focus lens 240 has been driven. The acquired image is stored in the storage section 327 through the image acquisition section 310 and the luminance signal calculation section 323.

The lens driver section 250 then drives the focus lens 240 to a second focus lens position that differs from the focus lens position at which the first image has been acquired, and the image sensor 260 acquires the second image of the object at the focus lens position to which the focus lens 240 has been driven. The second image thus acquired is output to the distance information acquisition section 320 through the image acquisition section 310.

When the second image has been acquired, the defocus parameter is calculated. The difference calculation section 324 included in the distance information acquisition section 320 reads the luminance signals Y of the first image from the storage section 327, and calculates the difference between the luminance signal Y of the first image and the luminance signal Y of the second image output from the luminance signal calculation section 323.

The second derivative calculation section 325 calculates the second derivative of the luminance signals Y of the second image output from the luminance signal calculation section 323. The second derivative calculation section 325 then reads the luminance signals Y of the first image from the storage section 327, and calculates the second derivative of the luminance signals Y. The second derivative calculation section 325 then calculates the average value of the second derivative of the first image and the second derivative of the second image.

The defocus parameter calculation section 326 calculates the defocus parameter by dividing the difference calculated by the difference calculation section 324 by the average value of the second derivatives calculated by the second derivative calculation section 325.

The defocus parameter has a linear relationship with the reciprocal of the object distance, and the object distance and the focus lens position have a one-to-one relationship. Therefore, the defocus parameter and the focus lens position have a one-to-one relationship. The relationship between the defocus parameter and the focus lens position is stored in the LUT storage section 328 in the form of a table. The distance information that corresponds to the object distance is represented by the focus lens position. Therefore, the defocus parameter calculation section 326 calculates the object distance to the optical system from the defocus parameter by linear interpolation using the defocus parameter and the information included in the table stored in the LUT storage section 328. The defocus parameter calculation section 326 thus calculates the object distance that corresponds to the defocus parameter. The calculated object distance is output to the concavity-convexity information acquisition section 380 as the distance information.

Note that the distance information need not necessarily be acquired using the above distance information acquisition process. For example, the distance information may be acquired using a stereo matching process. In this case, the imaging section 200 includes an optical system that captures a left image and a right image (that form a parallax image). The distance information acquisition section 320 performs a block matching process on the left image (reference image) and the right image with respect to the processing target pixel and its peripheral area (i.e., a block having a given size) using an epipolar line to calculate parallax information, and converts the parallax information into the distance information. This conversion process includes a process that corrects the optical magnification of the imaging section 200. The distance information thus obtained is output to the concavity-convexity information acquisition section 380 as the distance map (having the same pixel size as that of the stereo image in a narrow sense).

The distance information may be calculated by a Time-of-Flight method that utilizes infrared light or the like. When using the Time-of-Flight method, blue light may be used instead of infrared light, for example.

### 3. Second Embodiment

### 3.1. Image Processing Section

A second embodiment is described below. In the second embodiment, a concavity-convexity part is determined using the extracted concavity-convexity information in the same manner as in the first embodiment. The second embodiment differs from the first embodiment in that the exclusion target for which the enhancement process is omitted (or suppressed) is determined instead of a mucous membrane.

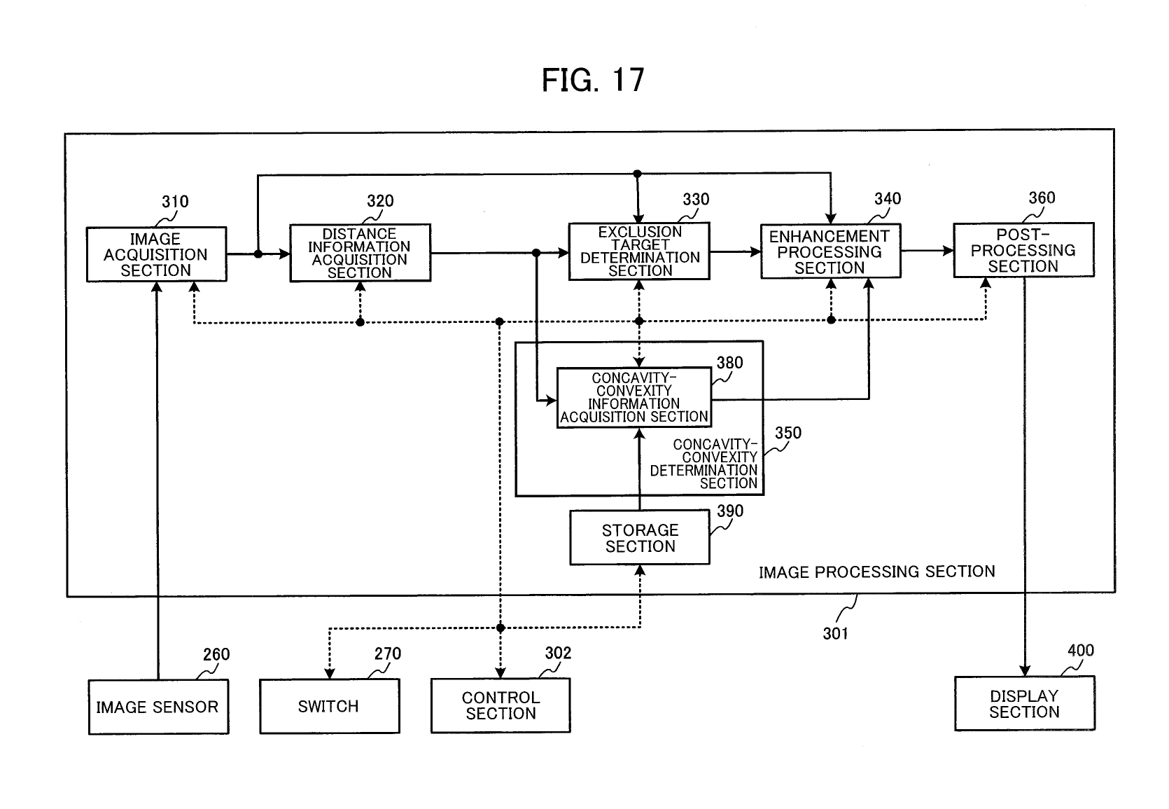

An endoscope apparatus according to the second embodiment may be configured in the same manner as the endoscope apparatus according to the first embodiment. FIG. 17 illustrates a configuration example of an image processing section 301 according to the second embodiment. The image processing section 301 includes an image acquisition section 310, a distance information acquisition section 320, an exclusion target determination section 330, an enhancement processing section 340, a post-processing section 360, a concavity-convexity information acquisition section 380, and a storage section 390. Note that the same elements as those described above in connection with the first embodiment are indicated by the same reference symbols, and description thereof is appropriately omitted.

The image acquisition section 310 is connected to the distance information acquisition section 320, the exclusion target determination section 330, and the enhancement processing section 340. The distance information acquisition section 320 is connected to the exclusion target determination section 330 and the concavity-convexity information acquisition section 380. The exclusion target determination section 330 is connected to the enhancement processing section 340. The control section 302 is bidirectionally connected to each section of the image processing section 301, and controls each section of the image processing section 301.

The exclusion target determination section 330 determines the exclusion target within the endoscopic image for which the enhancement process is omitted (or suppressed), based on the endoscopic image output from the image acquisition section 310 and the distance information output from the distance information acquisition section 320. The details of the exclusion target determination section 330 are described later.

The enhancement processing section 340 performs the enhancement process on the endoscopic image based on the extracted concavity-convexity information output from the concavity-convexity information acquisition section 380, and outputs the resulting endoscopic image to the post-processing section 360. The enhancement processing section 340 omits (or suppresses) the enhancement process on the exclusion target determined by the exclusion target determination section 330. The enhancement process may be performed while continuously changing the enhancement level at the boundary between the exclusion target area and an area other than the exclusion target area in the same manner as in the first embodiment. An enhancement process that simulates dye spraying (see the first embodiment) is performed as the enhancement process, for example. Specifically, the enhancement processing section 340 includes the dimensional information acquisition section 601 and the concavity extraction section 602 illustrated in FIG. 11, extracts a groove area from the surface of tissue, and performs a B component enhancement process on the groove area. Note that the configuration according to the second embodiment is not limited thereto. Various enhancement processes such as a structure enhancement process may also be used.

### 3.2. Exclusion Target Determination Process

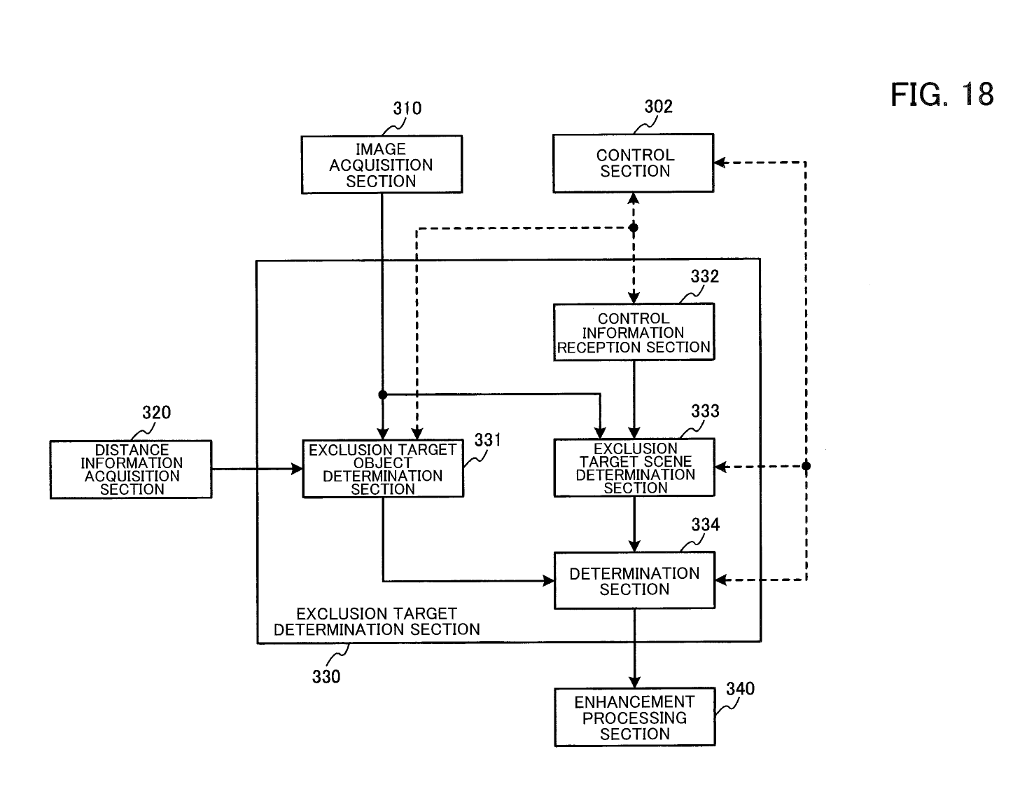

FIG. 18 illustrates a detailed configuration example of the exclusion target determination section 330. The exclusion target determination section 330 includes an exclusion target object determination section 331, a control information reception section 332, an exclusion target scene determination section 333, and a determination section 334.

The exclusion target object determination section 331 is connected to the determination section 334. The control information reception section 332 is connected to the exclusion target scene determination section 333. The exclusion target scene determination section 333 is connected to the determination section 334. The determination section 334 is connected to the enhancement processing section 340.

The exclusion target object determination section 331 determines whether or not each pixel of the endoscopic image is the exclusion target based on the endoscopic image output from the image acquisition section 310 and the distance information output from the distance information acquisition section 320. The exclusion target object determination section 331 determines a set of pixels that have been determined to be the exclusion target (hereinafter may be referred to as “exclusion target pixels”) to be the exclusion target object within the endoscopic image. Note that the exclusion target object is part of the exclusion target, and the exclusion target also includes the exclusion target scene described later.

The control information reception section 332 extracts control information for controlling the exclusion target-related function of the endoscope from the control signal output from the control section 302, and transmits the extracted control information to the exclusion target scene determination section 333. The term “control information” used herein refers to control information about the execution state of the function of the endoscope by which the exclusion target scene (described later) may occur. For example, the control information is ON/OFF control information about the water supply function of the endoscope.

The exclusion target scene determination section 333 determines an endoscopic image for which the enhancement process is omitted (or suppressed), based on the endoscopic image output from the image acquisition section 310 and the control information output from the control information reception section 332. The enhancement process on the entirety of the determined endoscopic image is omitted (or suppressed).

The determination section 334 determines the exclusion target within the endoscopic image based on the determination results of the exclusion target object determination section 331 and the determination results of the exclusion target scene determination section 333. Specifically, when it has been determined that the endoscopic image corresponds to the exclusion target scene, the determination section 334 determines the entire endoscopic image to be the exclusion target. When it has been determined that the endoscopic image does not correspond to the exclusion target scene, the determination section 334 determines a set of the exclusion target pixels to be the exclusion target. The determination section 334 transmits information about the determined exclusion target to the enhancement processing section 340.

### 3.3. Exclusion Target Object Determination Process

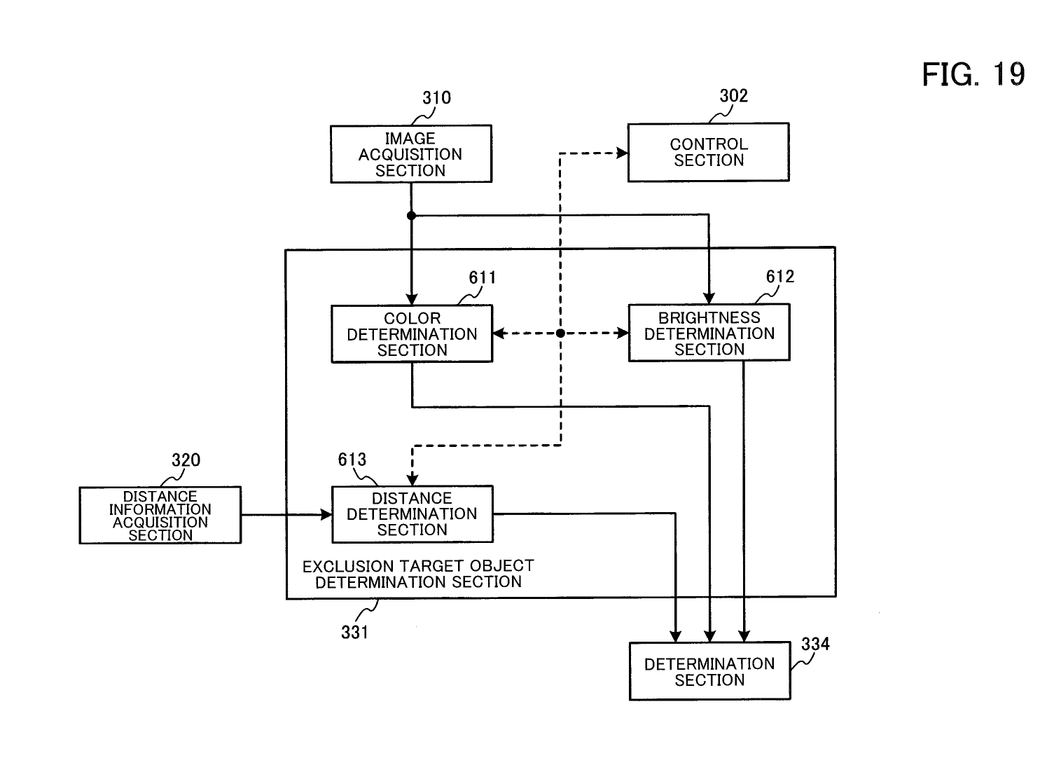

FIG. 19 illustrates a detailed configuration example of the exclusion target object determination section 331. The exclusion target object determination section 331 includes a color determination section 611, a brightness determination section 612, and a distance determination section 613.

The image acquisition section 310 transmits the endoscopic image to the color determination section 611 and the brightness determination section 612. The distance information acquisition section 320 transmits the distance information to the distance determination section 613. The color determination section 611, the brightness determination section 612, and the distance determination section 613 are connected to the determination section 334. The control section 302 is bidirectionally connected to each section of the exclusion target object determination section 331, and controls each section of the exclusion target object determination section 331.

The color determination section 611 determines whether or not each pixel of the endoscopic image is the exclusion target pixel based on the color of each pixel of the endoscopic image. Specifically, the color determination section 611 determines whether or not each pixel of the endoscopic image is the exclusion target pixel by comparing the hue of each pixel of the endoscopic image with a given hue that corresponds to the exclusion target object. The exclusion target object is a residue within the endoscopic image, for example. A residue within the endoscopic image is normally yellow. For example, when the hue H of the pixel satisfies the following expression (10), the pixel is determined to be the exclusion target pixel since the pixel corresponds to a residue.

30°<H≦50° (10)

Although an example in which the color determination section 611 determines a residue to be the exclusion target object has been described above, the exclusion target object is not limited to a residue. For example, the exclusion target object is an object within the endoscopic image other than a mucous membrane that has a characteristic color (e.g., metallic color (treatment tool)). Although an example in which the exclusion target object is determined based only on hue has been described above, the exclusion target object may be determined based on hue and chroma. When the determination target pixel is almost achromatic, it may be difficult to determine whether or not the determination target pixel corresponds to the exclusion target object in a stable manner since a significant change in hue may occur due to a small change in pixel value due to the effects of noise. In this case, it is possible to make a determination in a more stable manner by determining whether or not the determination target pixel corresponds to the exclusion target object based on hue and chroma.

It is possible to exclude an object other than a mucous membrane that has a characteristic color from the enhancement target by thus determining the exclusion target object based on color.

The brightness determination section 612 determines whether or not each pixel of the endoscopic image is the exclusion target pixel based on the brightness of each pixel of the endoscopic image. Specifically, the brightness determination section 612 determines whether or not each pixel of the endoscopic image is the exclusion target pixel by comparing the brightness of each pixel of the endoscopic image with a given brightness that corresponds to the exclusion target object. The exclusion target pixel is a blocked-up shadow area or a blown-out highlight area, for example. The term “blocked-up shadow area” used herein refers to an area of the endoscopic image for which it is difficult to improve the lesion detection accuracy through the enhancement process since the brightness is insufficient. The term “blown-out highlight area” used herein refers to an area of the endoscopic image in which a mucous membrane (enhancement target) is not captured since the pixel value is saturated. The brightness determination section 612 determines an area that satisfies the following expression (11) to be the blocked-up shadow area, and determines an area that satisfies the following expression (12) to be the blown-out highlight area.

Y<T_{low} (11)

Y>T_{high} (12)

Note that Y is the luminance value calculated by the expression (9). T_{low} is a given threshold value for determining the blocked-up shadow area, and T_{high} is a given threshold value for determining the blown-out highlight area. Note that the brightness is not limited to the luminance. The G pixel value may be used as the brightness, or the maximum value among the R pixel value, the G pixel value, and the B pixel value may be used as the brightness.

It is possible to exclude an area that does not contribute to an improvement in lesion detection accuracy from the enhancement target by thus determining the exclusion target pixel based on brightness.

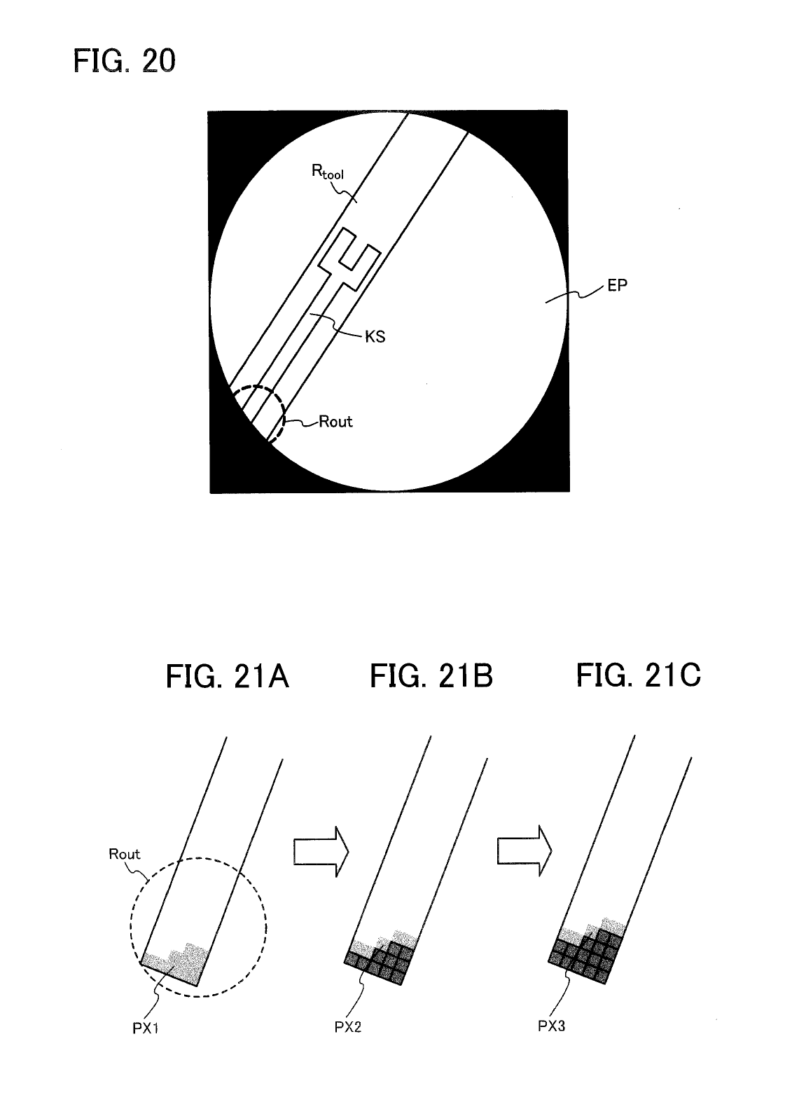

The distance determination section 613 determines whether or not each pixel of the endoscopic image is the exclusion target pixel based on the distance information about each pixel of the endoscopic image. The exclusion target object is a treatment tool, for example. As illustrated in FIG. 20, a treatment tool is present within an almost constant range (treatment tool area Rₜₒₒₗ) in an endoscopic image EP. Therefore, the distance information in a forceps channel neighborhood area Rout situated on the end of the imaging section 200 is known from the design information about the endoscope. Therefore, whether or not each pixel is the exclusion target pixel that corresponds to a treatment tool is determined as described below.

The distance determination section 613 determines whether or not a treatment tool has been inserted into the forceps channel. Specifically, the distance determination section 613 determines whether or not a treatment tool has been inserted into the forceps channel based on the number of pixels PX1 within the forceps channel neighborhood area Rout for which the distance satisfies the following expressions (13) and (14) (see FIG. 21A). When the number of pixels PX1 is equal to or larger than a given threshold value, the distance determination section 613 determines that a treatment tool has been inserted into the forceps channel. When it has been determined that a treatment tool has been inserted into the forceps channel, the pixels PX1 are determined (set) to be the exclusion target pixel.

D(x, y)<T_{dist} (13)

(x, y)∈Rₒᵤₜ (14)

Note that D(x, y) is the distance (i.e., the value of the distance map) corresponding to the pixel situated at coordinates (x, y). T_{dist} is a distance threshold value in the forceps channel neighborhood area Rout. The distance threshold value T_{dist} is set based on the design information about the endoscope. The expression (14) represents that the pixel situated at coordinates (x, y) is situated within the forceps channel neighborhood area Rout in the endoscopic image.

The distance determination section 613 then determines pixels PX2 that are situated adjacent to the exclusion target pixels and satisfies the following expressions (15) and (16) to be the exclusion target pixel (see FIG. 21B).

|D(x, y)−Dᵣₑₘₒᵥₑ(p, q)|<T_{neighbor} (15)

(x, y)∈Rₜₒₒₗ (16)

Note that Dᵣₑₘₒᵥₑ(p, q) is the distance (i.e., the value of the distance map) corresponding to the exclusion target pixel situated adjacent to the pixel situated at coordinates (x, y), and (p, q) is the coordinates of the exclusion target pixel. The expression (16) represents that the pixel situated at coordinates (x, y) is situated within the treatment tool area Rₜₒₒₗ in the endoscopic image. T_{neighbor} is a threshold value for the difference between the distance corresponding to the pixel situated within the treatment tool area Rₜₒₒₗ and the distance corresponding to the exclusion target pixel.

The distance determination section 613 repeatedly performs the above determination process (see FIG. 21C). The distance determination section 613 terminates the determination process when a pixel PX3 that satisfies the expressions (15) and (16) is not present, or when the number of exclusion target pixels has become equal to or larger than a given number.

The determination process termination condition is described below. When a treatment tool comes in contact with tissue, pixels that correspond to tissue also satisfy the expressions (15) and (16), and the number of exclusion target pixels may reach the number of pixels included in the treatment tool area Rₜₒₒₗ. The maximum number of pixels of the endoscopic image that correspond to a treatment tool is known from the diameter and the maximum length of the treatment tool. It is possible to suppress a situation in which a pixel is determined to be the exclusion target pixel due to a factor other than a treatment tool by utilizing the maximum number of pixels as the determination process termination condition.

Note that the termination condition is not limited thereto. For example, the determination process may be terminated when it has been determined that the exclusion determination pixels do not correspond to the shape of a treatment tool using a known technique such as a template matching technique.

Although an example has been described above in which each element of the exclusion target object determination section 331 determines the exclusion target pixel using a different determination standard (i.e., color, brightness, or distance), the configuration is not limited thereto. The exclusion target object determination section 331 may determine the exclusion target pixel using a plurality of determination standards in combination. An example in which a bleeding area is determined to be the exclusion target object is described below. A bleeding area within the endoscopic image is in the color of blood. The surface of a bleeding area is almost flat. Therefore, a bleeding area can be determined to be the exclusion target object by causing the color determination section 611 to determine whether or not the color of blood is captured, and causing the distance determination section 613 to determine the degree of flatness of the surface of the corresponding area. Note that the degree of flatness of the surface of the corresponding area is determined by locally adding up the absolute values of the extracted concavity-convexity information, for example. It is determined that the surface of the corresponding area is flat when the local sum of the absolute values of the extracted concavity-convexity information is small.

It is possible to exclude a treatment tool from the enhancement target by thus determining the presence of a treatment tool based on the position of the forceps channel and the continuity of pixels on the distance map that correspond to a treatment tool.

According to the second embodiment, the exclusion target determination section 330 determines an area for which the feature quantity based on the pixel value of the captured image satisfies a given condition that corresponds to the exclusion target, to be the exclusion target area. More specifically, the exclusion target determination section 330 determines an area for which color information (e.g., hue value) (i.e., feature quantity) satisfies a given condition (e.g., a color range that corresponds to a residue, or a color range that corresponds to a treatment tool) relating to the color of the exclusion target, to be the exclusion target area.

According to the second embodiment, the exclusion target determination section 330 determines an area for which brightness information (e.g., luminance value) (i.e., feature quantity) satisfies a given condition (e.g., a brightness range that corresponds to the blocked-up shadow area, or a brightness range that corresponds to the blown-out highlight area) relating to the brightness of the exclusion target, to be the exclusion target area.

This makes it possible to determine an object that should not be enhanced based on the feature quantity of the image. Specifically, it is possible to determine an object that should not be enhanced by setting the exclusion target feature using a feature quantity condition, and detecting an area that satisfies the condition. Note that the color information is not limited to a hue value. For example, various other color index values (e.g., chroma) may also be used as the color information. The brightness information is not limited to a luminance value. For example, various other brightness index values (e.g., G pixel value) may also be used as the brightness information.

According to the second embodiment, the exclusion target determination section 330 determines an area for which the distance information satisfies a given condition relating to the exclusion target distance to be the exclusion target area. Specifically, the exclusion target determination section 330 determines an area in which the distance to the object represented by the distance information continuously changes (e.g., an area of forceps captured within the captured image), to be the exclusion target area.

This makes it possible to determine an object that should not be enhanced based on distance. Specifically, it is possible to determine an object that should not be enhanced by setting the exclusion target feature using a distance condition, and detecting an area that satisfies the condition. Note that the exclusion target object that is determined using the distance information is not limited to forceps, but may be another treatment tool that may be captured within the captured image.

### 3.4. Exclusion Target Scene Determination Process

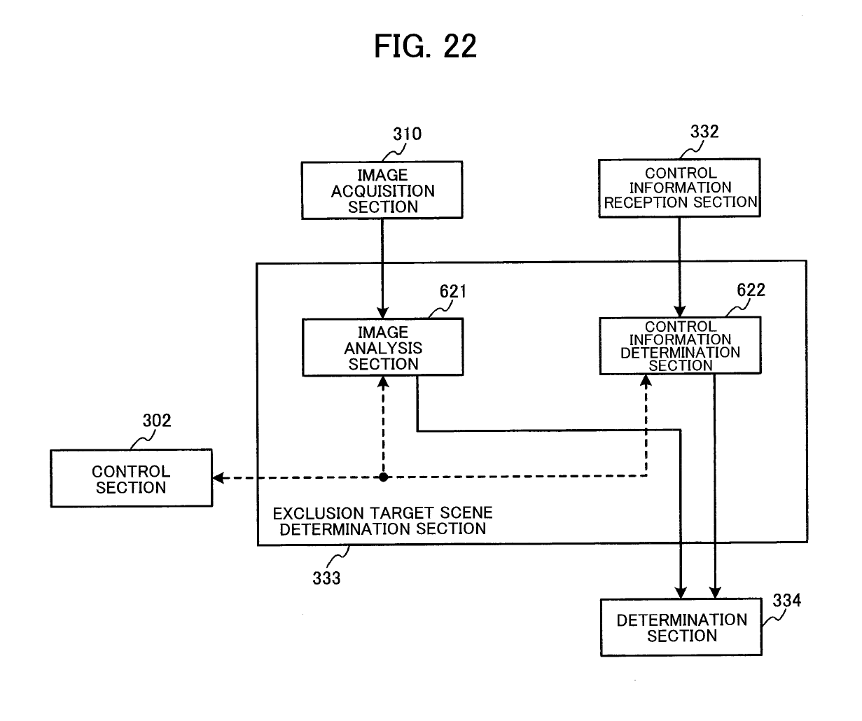

FIG. 22 illustrates a detailed configuration example of the exclusion target scene determination section 333. The exclusion target scene determination section 333 includes an image analysis section 621 and a control information determination section 622. The exclusion target scene determination section 333 determines that the determination target scene is the exclusion target scene when the image analysis section 621 or the control information determination section 622 has determined that the determination target scene is the exclusion target scene.

The image acquisition section 310 transmits the endoscopic image to the image analysis section 621. The control information reception section 332 transmits the extracted control information to the control information determination section 622.

The image analysis section 621 is connected to the determination section 334. The control information determination section 622 is connected to the determination section 334.

The image analysis section 621 analyzes the endoscopic image, and determines whether or not the endoscopic image is an image that captures the exclusion target scene. The exclusion target scene is a water supply scene, for example. Since almost the entirety of the endoscopic image is covered by water during a water supply operation, an object that is useful for detecting a lesion is not captured within the endoscopic image, and it is unnecessary to perform the enhancement process.

The image analysis section 621 calculates the image feature quantity from the endoscopic image, and compares the calculated image feature quantity with the image feature quantity stored in the control section 302. The image analysis section 621 determines that the determination target scene is a water supply scene when the similarity between the calculated image feature quantity and the image feature quantity stored in the control section 302 is equal to or larger than a given value. The image feature quantity stored in the control section 302 is a feature quantity calculated from an endoscopic image during a water supply operation. For example, the image feature quantity stored in the control section 302 is a Haar-like feature quantity. The details of the Haar-like feature quantity are described in Takeshi MITA, Toshimitsu KANEKO, and Osamu HORI (2006), “Joint Haar-like Features Based on Feature Co-occurrence for Face Detection”, The transactions of the Institute of Electronics, Information and Communication Engineers, D, Vol. J89-D, No. 8, pp. 1791-1801, for example. Note that the image feature quantity is not limited to the Haar-like feature quantity. A known image feature quantity other than the Haar-like feature quantity may also be used.

The exclusion target scene is not limited to a water supply scene, but may be a scene in which an object that is useful for detecting a lesion is not captured within the endoscopic image (e.g., when mist (i.e., smoke generated when cauterizing tissue) is produced). It is possible to suppress a situation in which the enhancement process is unnecessarily performed, by determining whether or not the determination target scene is the exclusion target scene based on the endoscopic image.

The control information determination section 622 determines whether or not the determination target scene is the exclusion target scene based on the control information output from the control information reception section 332. For example, the control information determination section 622 determines that the determination target scene is the exclusion target scene when the control information that represents that the water supply function is enabled has been input. Note that the control information determination section 622 determines that the determination target scene is the exclusion target scene only when the control information that represents that the water supply function is enabled has been input. For example, the control information determination section 622 may determine that the determination target scene is the exclusion target scene when the control information that represents that a function is enabled that causes a situation in which an object that is useful for detecting a lesion is not captured within the endoscopic image (e.g., the control information that represents that an IT knife function that produces mist is enabled) has been input.

Although an example has been described above in which the exclusion target scene determination section 333 determines that the determination target scene is the exclusion target scene when the image analysis section 621 or the control information determination section 622 has determined that the determination target scene is the exclusion target scene, the configuration is not limited thereto. For example, the exclusion target scene determination section 333 may determine whether or not the determination target scene is the exclusion target scene by combining the determination result of the image analysis section 621 and the determination result of the control information determination section 622. For example, even when the IT knife function that may produce mist has been enabled, an object that should be enhanced is captured within the endoscopic image when the IT knife does not come in contact with tissue, or when the amount of smoke generated is small. In such a case, it is desirable to perform the enhancement process. However, since the control information determination section 622 determines that the determination target scene is the exclusion target scene when the IT knife function that may produce mist has been enabled, the enhancement process is not performed. Therefore, it is desirable to determine that the determination target scene is the exclusion target scene when both the image analysis section 621 and the control information determination section 622 have determined that the determination target scene is the exclusion target scene. Specifically, it is desirable to determine whether or not the determination target scene is the exclusion target scene by optimally combining the determination result of the image analysis section 621 and the determination result of the control information determination section 622 corresponding to the exclusion target scene.

It is possible to suppress a situation in which the enhancement process is unnecessarily performed, by thus determining whether or not the determination target scene is the exclusion target scene based on the function that may produce the exclusion target scene.

According to the second embodiment, the exclusion target within the endoscopic image for which the enhancement process is not performed is determined based on the endoscopic image and the distance information, and the concavity-convexity information about the surface of the object is enhanced with respect to an area other than the exclusion target based on the distance information. Since the enhancement process on an area for which the enhancement process is unnecessary can thus be omitted (or suppressed), it is possible to improve the capability to discriminate between an area for which the enhancement process is necessary, and an area for which the enhancement process is unnecessary, and suppress as much as possible a situation in which the user gets tired when observing the image as compared with the case where the enhancement process is also performed on an area for which the enhancement process is unnecessary.

According to the second embodiment, the exclusion target determination section 330 includes the control information reception section 332 that receives the control information about the endoscope apparatus, and determines the captured image to be the exclusion target area when the control information received by the control information reception section 332 is given control information (e.g., water supply instruction information, or IT knife enable instruction information) that corresponds to the exclusion target scene that is the exclusion target.

This makes it possible to determine an image that corresponds to a scene that should not be enhanced based on the control information about the endoscope apparatus. Specifically, it is possible to determine an image that corresponds to a scene that should not be enhanced by setting the control information that produces the exclusion target scene as a condition, and detecting the control information that satisfies the condition. This makes it possible to disable the enhancement process when the observation target object is not captured (i.e., the enhancement process is performed only when it is necessary), and provide an image appropriate for a medical examination to the user.

### 4. Third Embodiment

### 4.1. Image Processing Section

A third embodiment illustrates an example in which a process that classifies concavity-convexity parts of the object into specific types or states is performed as the process that determines a concavity-convexity part of the object. The scale and the size of the classification target concavity-convexity part may differ from, or be almost the same as, those of the first and second embodiments. In the first and second embodiments, folds, a polyp, or the like present on a mucous membrane is extracted. In the third embodiment, a small pit pattern present on the surface of a mucous membrane is classified.

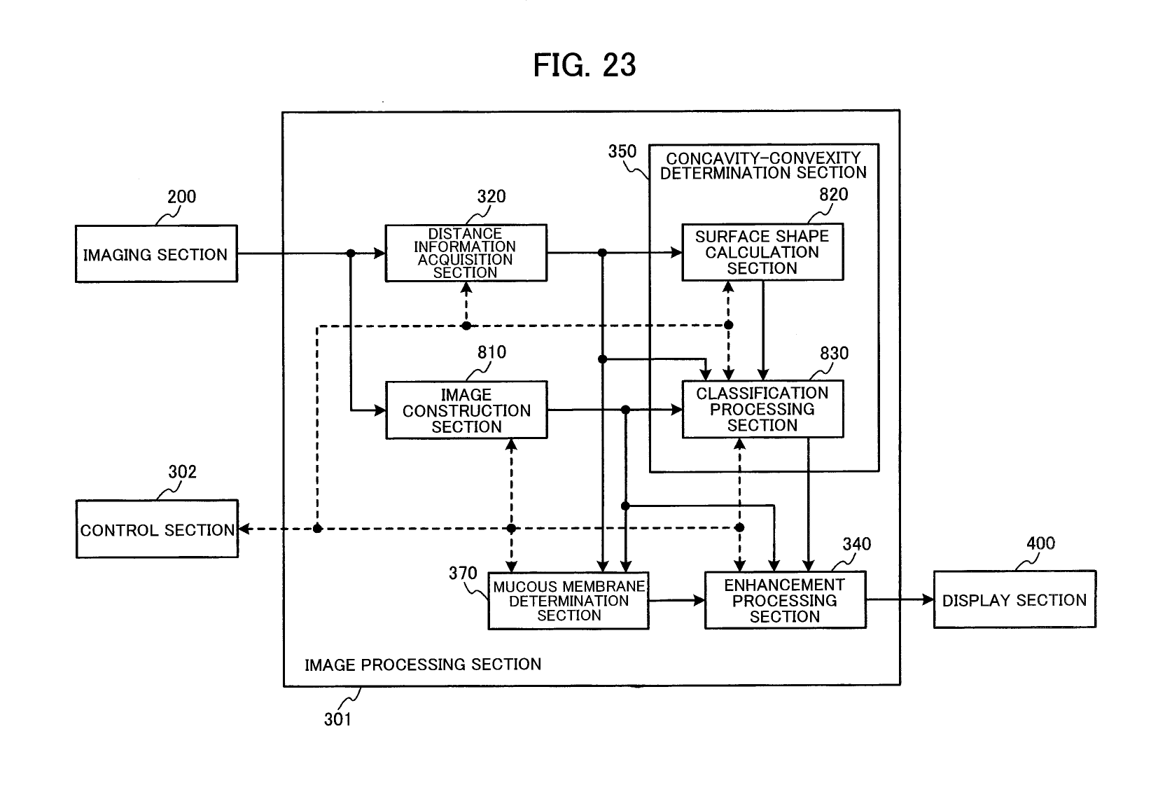

FIG. 23 illustrates a configuration example of an image processing section 301 according to the third embodiment. The image processing section 301 includes a distance information acquisition section 320, an enhancement processing section 340, a concavity-convexity determination section 350, a mucous membrane determination section 370, and an image construction section 810. The concavity-convexity determination section 350 includes a surface shape calculation section 820 (three-dimensional shape calculation section) and a classification processing section 830. An endoscope apparatus according to the third embodiment may be configured in the same manner as in FIG. 3. Note that the same elements as those described above in connection with the first and second embodiments are indicated by the same reference symbols, and description thereof is appropriately omitted.

The image construction section 810 is connected to the classification processing section 830, the mucous membrane determination section 370, and the enhancement processing section 340. The distance information acquisition section 320 is connected to the surface shape calculation section 820, the classification processing section 830, and the mucous membrane determination section 370. The surface shape calculation section 820 is connected to the classification processing section 830. The classification processing section 830 is connected to the enhancement processing section 340. The mucous membrane determination section 370 is connected to the enhancement processing section 340. The enhancement processing section 340 is connected to the display section 400. The control section 302 is bidirectionally connected to each section of the image processing section 301, and controls each section of the image processing section 301. The control section 302 outputs the optical magnification stored in the memory 211 of the imaging section 200 to the image processing section 301.

The image construction section 810 acquires the captured image output from the imaging section 200, performs image processing on the captured image so that the captured image can be output from (displayed on) the display section 400. For example, when the imaging section 200 includes an A/D conversion section (not illustrated in the drawings), the image construction section 810 performs an OB process, a gain process, a y process, and the like on a digital image output from the A/D conversion section. The image construction section 810 outputs the resulting image to the classification processing section 830, the mucous membrane determination section 370, and the enhancement processing section 340.

The concavity-convexity determination section 350 performs a classification process on pixels that correspond to a structure within the image based on the distance information and a classification reference. Note that the details of the classification process are described later. An outline of the classification process is described below.

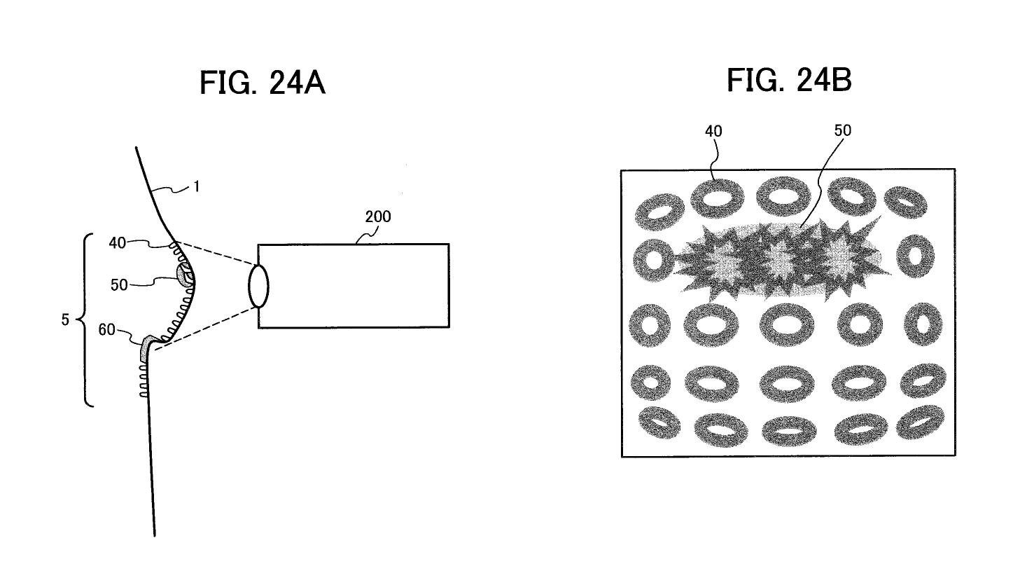

FIG. 24A illustrates the relationship between the imaging section 200 and the object when observing an abnormal area (e.g., early lesion). FIG. 24B illustrates an example of an image acquired when observing the abnormal area. A normal duct 40 represents a normal pit pattern, an abnormal duct 50 represents an abnormal pit pattern having a concavity-convexity shape, and a duct disappearance area 60 (recessed lesion) represents an abnormal area in which the pit pattern has disappeared due to a lesion. The normal duct 40 is a structure that is classified as a normal area, and the abnormal duct 50 and the duct disappearance area 60 are structures that are classified as an abnormal area (non-normal area). Note that the term “normal area” refers to a structure that is not likely to be a lesion, and the term “abnormal area” refers to a structure that is likely to be a lesion.

When the operator has found an abnormal area (see FIG. 24A), the operator brings the imaging section 200 closer to the abnormal area so that the imaging section 200 directly faces the abnormal area as much as possible. As illustrated in FIG. 24B, a normal area has a pit pattern in which regular structures are uniformly arranged. Such a normal area can be detected by image processing by registering or learning a normal pit pattern structure as the known characteristic information (prior information), and performing a matching process or the like. Since the pit pattern in an abnormal area has a concavity-convexity shape, or has a missing part, the pit pattern in an abnormal area has various shapes as compared with a normal area. Therefore, it is difficult to detect an abnormal area based on the known characteristic information. In the third embodiment, the pit pattern is classified into a normal area and an abnormal area by classifying an area that has not been detected as a normal area as an abnormal area. It is possible to prevent a situation in which an abnormal area is missed, and improve the qualitative diagnosis accuracy by enhancing an abnormal area classified in this manner.

Specifically, the surface shape calculation section 820 calculates a normal vector to the surface of the object corresponding to each pixel of the distance map as surface shape information (three-dimensional shape information in a broad sense). The classification processing section 830 projects a reference pit pattern (classification reference) onto the surface of the object based on the normal vector. The classification processing section 830 adjusts the size of the reference pit pattern to the size within the image (i.e., an apparent size that decreases within the image as the distance increases) based on the distance at the corresponding pixel position. The classification processing section 830 performs a matching process on the corrected reference pit pattern and the image to detect an area that agrees with the reference pit pattern.

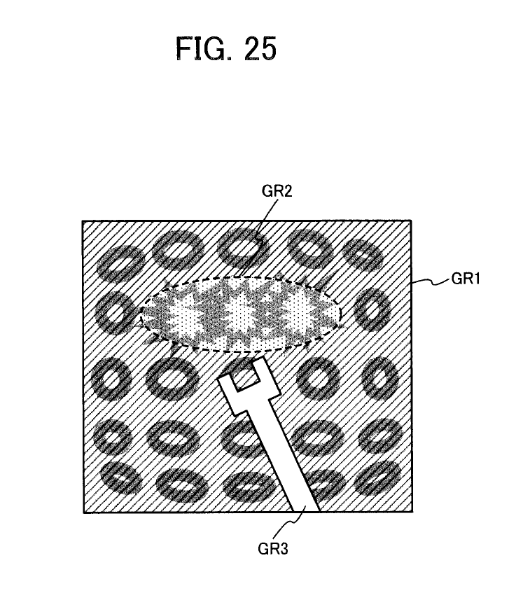

As illustrated in FIG. 25, the classification processing section 830 uses the shape of a normal pit pattern as the reference pit pattern, classifies an area GR1 that agrees with the reference pit pattern as a “normal area”, and classifies areas GR2 and GR3 that do not agree with the reference pit pattern as an “abnormal area (non-normal area)”, for example. The area GR3 is an area in which a treatment tool (e.g., forceps or surgical knife) is captured, for example. The area GR3 is classified as the abnormal area since a pit pattern is not captured in the area GR3.

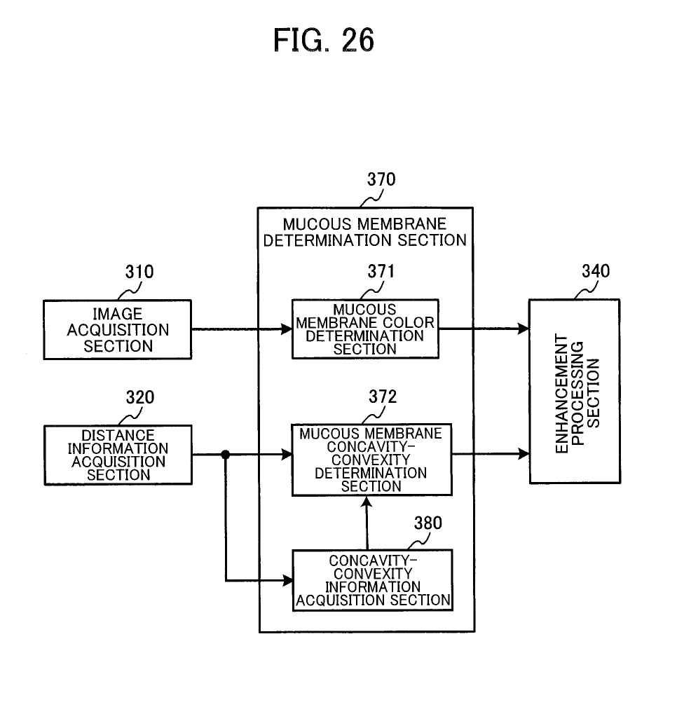

The mucous membrane determination section 370 includes a mucous membrane color determination section 371, a mucous membrane concavity-convexity determination section 372, and a concavity-convexity information acquisition section 380 (see FIG. 26). In the third embodiment, the concavity-convexity information acquisition section 380 extracts the concavity-convexity information for determining a mucous membrane based on concavities-convexities (e.g., groove) instead of determining a concavity-convexity part for implementing the enhancement process. The operation of the mucous membrane color determination section 371, the mucous membrane concavity-convexity determination section 372, and the concavity-convexity information acquisition section 380 is the same as described above in connection with the first embodiment, and description thereof is omitted.

The enhancement processing section 340 performs the enhancement process on the image of an area that has been determined by the mucous membrane determination section 370 to be a mucous membrane, and classified by the classification processing section 830 as the abnormal area, and outputs the resulting image to the display section 400. In the example illustrated in FIG. 25, the areas GR1 and GR2 are determined to be a mucous membrane, and the areas GR2 and GR3 are classified as the abnormal area. Specifically, the enhancement process is performed on the area GR2. For example, the enhancement processing section 340 performs a filtering process or a color r that enhances the structure of the pit pattern on the area GR2 that is a mucous membrane and is the abnormal area.

Note that the enhancement process is not limited thereto, but may be another process that enhances or differentiates a specific target within the image. For example, the enhancement process may be a process that enhances an area classified as a specific type or state, a process that encloses an area classified as a specific type or state with a line, or a process that adds a mark that represents an area classified as a specific type or state. A process that applies a specific color may be performed on an area (e.g., the areas GR1 and GR3 in the example illustrated in FIG. 25) other than a specific area to enhance (or differentiate) the specific area (GR2).

According to the third embodiment, the concavity-convexity determination section 350 includes the surface shape calculation section 820 that calculates the surface shape information about the object based on the distance information and the known characteristic information, and the classification processing section 830 that generates the classification reference based on the surface shape information, and performs the classification process that utilizes the generated classification reference. The concavity-convexity determination section 350 performs the classification process that utilizes the classification reference as the concavity-convexity determination process.

This makes it possible to perform the enhancement process on only a structure that has been determined to be a mucous membrane and classified as the abnormal area. Therefore, even when an object without a pit pattern (e.g., treatment tool) has been classified as the abnormal area, the object that is not a mucous membrane is not enhanced. It is possible to assist in a qualitative lesion/non-lesion diagnosis by thus enhancing only a structure that is likely to be a lesion.

### 4.2. First Modification

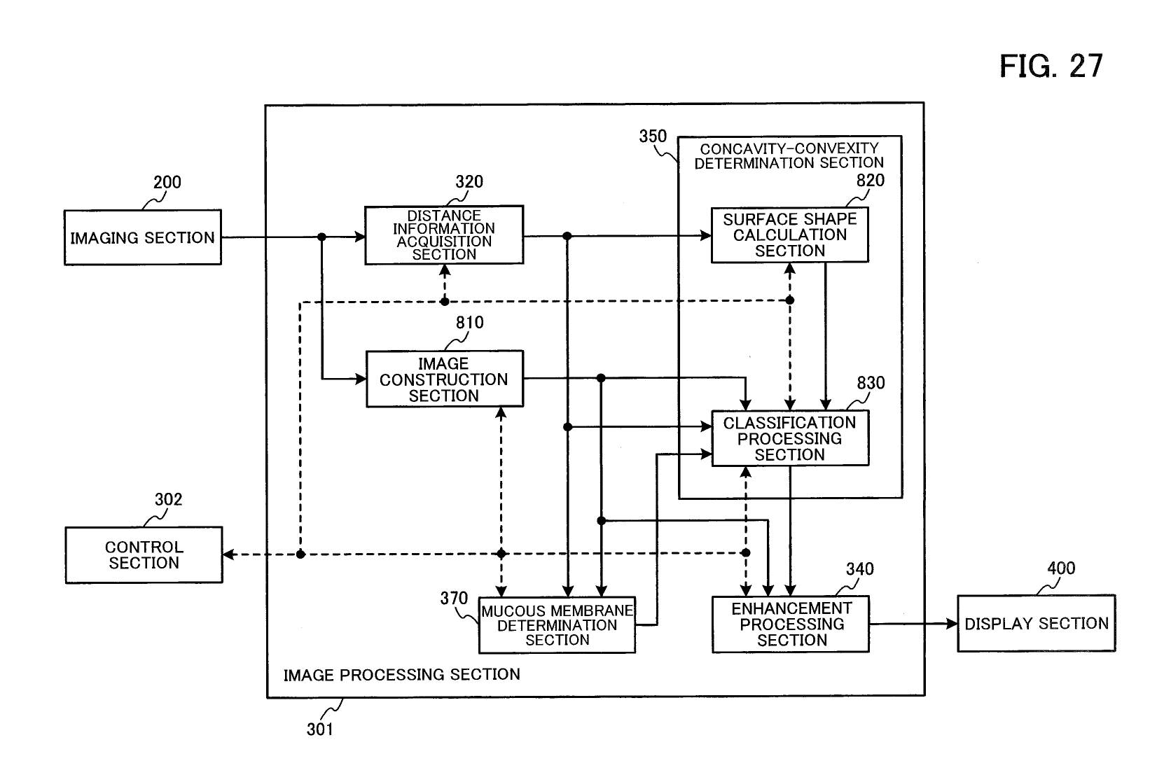

FIG. 27 illustrates a configuration example of an image processing section 301 according to a first modification of the third embodiment. The image processing section 301 includes a distance information acquisition section 320, an enhancement processing section 340, a concavity-convexity determination section 350, a mucous membrane determination section 370, and an image construction section 810. The concavity-convexity determination section 350 includes a surface shape calculation section 820 and a classification processing section 830. Note that the same elements as those described above with reference to FIG. 23 are indicated by the same reference symbols, and description thereof is appropriately omitted.

The mucous membrane determination section 370 is connected to the classification processing section 830. The classification processing section 830 is connected to the enhancement processing section 340. Specifically, while the mucous membrane determination process and the classification process are performed in parallel in the configuration example illustrated in FIG. 23, the classification process is performed directly after the mucous membrane determination process in the first modification. More specifically, the classification processing section 830 performs the classification process on the image of an area (e.g., the areas GR1 and GR2 in FIG. 25) that has been determined by the mucous membrane determination section 370 to be a mucous membrane, to classify the area that has been determined to be a mucous membrane into the normal area (GR1) and the abnormal area (GR2). The enhancement processing section 340 performs the enhancement process on the image of the area (GR2) that has been classified by the classification processing section 830 as the abnormal area.

According to the first modification, the concavity-convexity determination section 350 performs the classification process on a mucous membrane area determined by the mucous membrane determination section 370.

This makes it possible to suppress a situation in which the enhancement process is performed on the abnormal area other than a mucous membrane in the same manner as the configuration example illustrated in FIG. 23. The calculation cost can be reduced by performing the classification process only on an area that has been determined to be a mucous membrane. It is also possible to improve the accuracy of the classification reference by generating the classification reference corresponding to only on an area that has been determined to be a mucous membrane.

### 4.3. Second Modification

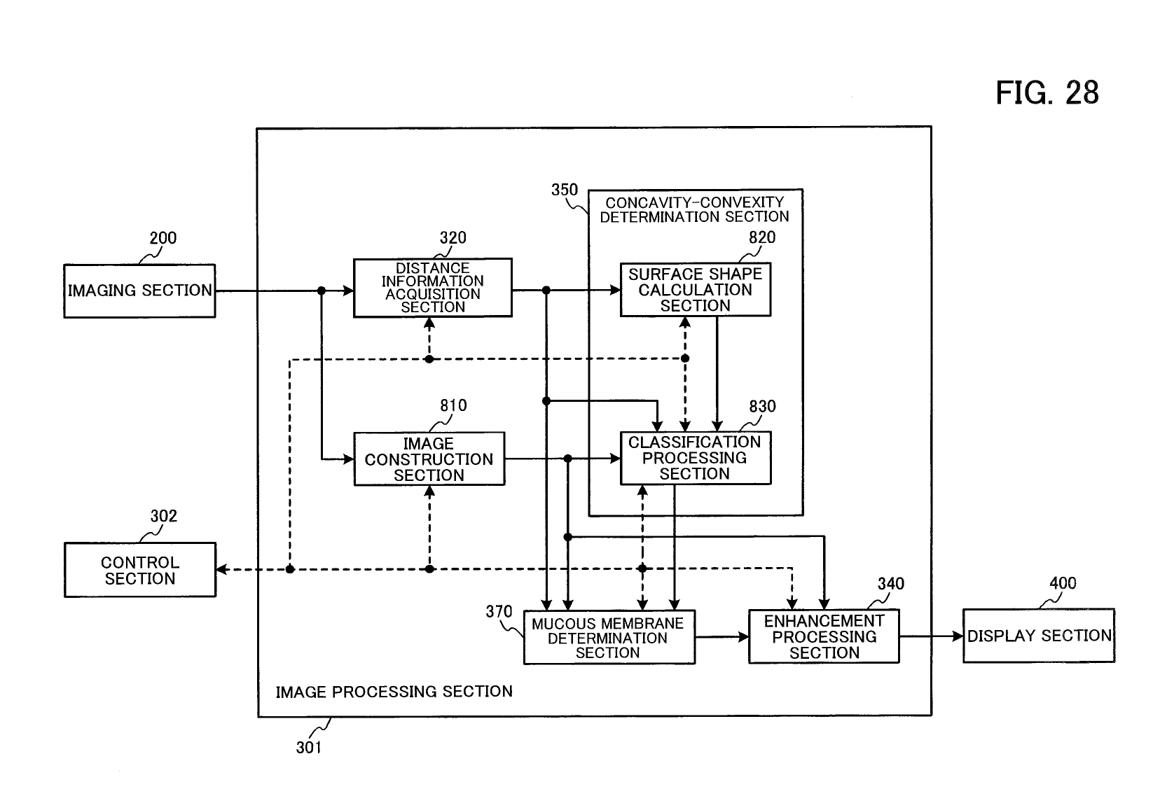

FIG. 28 illustrates a configuration example of an image processing section 301 according to a second modification of the third embodiment. The image processing section 301 includes a distance information acquisition section 320, an enhancement processing section 340, a concavity-convexity determination section 350, a mucous membrane determination section 370, and an image construction section 810. The concavity-convexity determination section 350 includes a surface shape calculation section 820 and a classification processing section 830. Note that the same elements as those described above with reference to FIG. 23 are indicated by the same reference symbols, and description thereof is appropriately omitted.

The classification processing section 830 is connected to the mucous membrane determination section 370. The mucous membrane determination section 370 is connected to the enhancement processing section 340. Specifically, the mucous membrane determination process is performed directly after the classification process in the second modification. More specifically, the mucous membrane determination section 370 performs the mucous membrane determination process on the image of an area (e.g., the areas GR2 and GR3 in FIG. 25) that has been classified by the classification processing section 830 as the abnormal area, and determines a mucous membrane area (GR2) from the area classified as the abnormal area. The enhancement processing section 340 performs the enhancement process on the image of the area (GR2) that has been determined by the mucous membrane determination section 370 to be a mucous membrane.

According to the second modification, the mucous membrane determination section 370 performs the process that determines a mucous membrane area on the object that has been classified by the classification process as a specific class (e.g., abnormal area).

This makes it possible to suppress a situation in which the enhancement process is performed on the abnormal area other than a mucous membrane in the same manner as the configuration example illustrated in FIG. 23. The calculation cost can be reduced by performing the mucous membrane determination process only on an area that has been classified as a specific class (e.g., abnormal area).

### 5. Fourth Embodiment

In a fourth embodiment, a pit pattern is classified into the normal area and the abnormal area in the same manner as in the third embodiment. The fourth embodiment differs from the third embodiment in that the exclusion target for which the enhancement process is omitted (or suppressed) is determined instead of a mucous membrane.

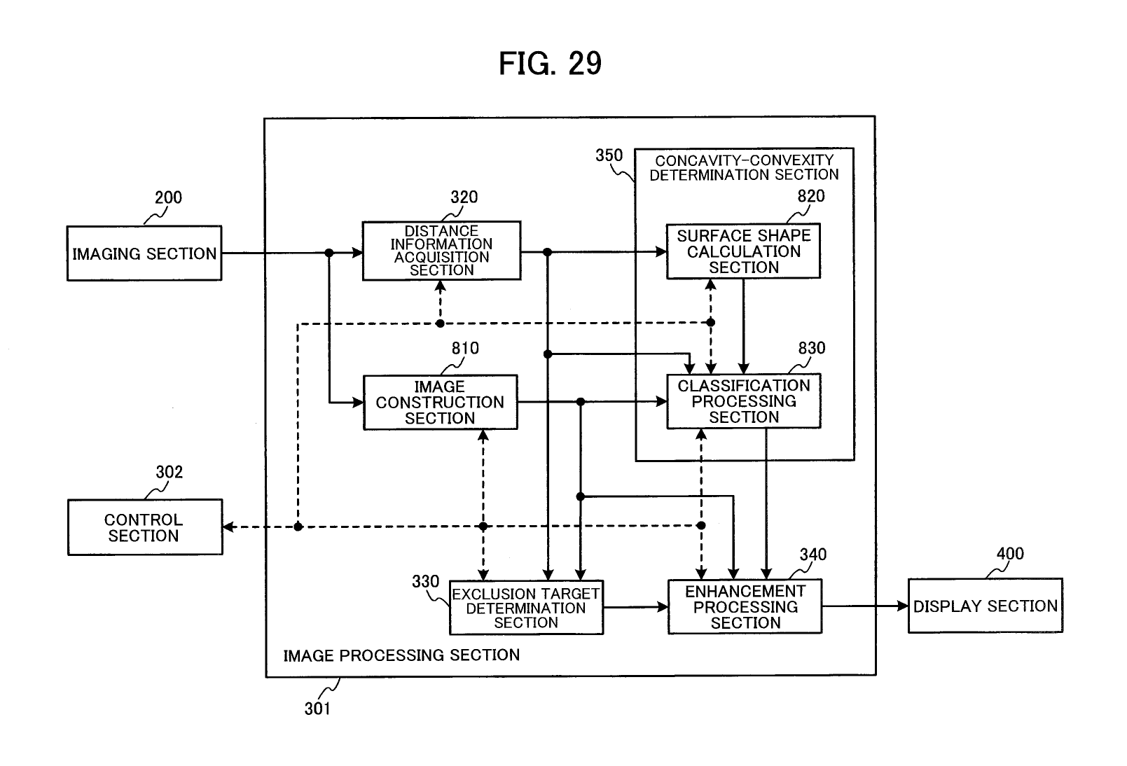

FIG. 29 illustrates a configuration example of an image processing section 301 according to the fourth embodiment. The image processing section 301 includes a distance information acquisition section 320, an enhancement processing section 340, a concavity-convexity determination section 350, an exclusion target determination section 330, and an image construction section 810. The concavity-convexity determination section 350 includes a surface shape calculation section 820 and a classification processing section 830. An endoscope apparatus according to the fourth embodiment may be configured in the same manner as in FIG. 3. Note that the same elements as those described above in connection with the third embodiment are indicated by the same reference symbols, and description thereof is appropriately omitted.

The image construction section 810 is connected to the classification processing section 830, the exclusion target determination section 330, and the enhancement processing section 340. The distance information acquisition section 320 is connected to the surface shape calculation section 820, the classification processing section 830, and the exclusion target determination section 330. The surface shape calculation section 820 is connected to the classification processing section 830. The classification processing section 830 is connected to the enhancement processing section 340. The exclusion target determination section 330 is connected to the enhancement processing section 340. The enhancement processing section 340 is connected to the display section 400. The control section 302 is bidirectionally connected to each section of the image processing section 301, and controls each section of the image processing section 301. The control section 302 outputs the information that is stored in the memory 211 of the imaging section 200 and relates to the execution state of the function of the endoscope (hereinafter referred to as “function information”) to the image processing section 301. Examples of the function of the endoscope include a water supply function that discharges water to the object to remove an obstruction to observation.

The exclusion target determination section 330 determines a specific object (e.g., residue, treatment tool, or blocked-up shadow area) or a specific scene (e.g., water supply or treatment using an IT knife) as the exclusion target in the same manner as in the second embodiment. The enhancement processing section 340 performs the enhancement process on an area (GR2) that is an area other than an area (e.g., the area GR3 in FIG. 25) that has been determined by the exclusion target determination section 330 to be the exclusion target, and has been classified by the classification processing section 830 as the abnormal area (GR2 and GR3). When a specific scene has been detected, the entire image is determined to be the exclusion target, and the enhancement process is not performed.

Note that the classification process may be performed directly after the exclusion target determination process in the same manner as in the third embodiment. Specifically, when detecting a specific object, the classification process may be performed on an image other than the specific object. When detecting a specific scene, the classification process may be performed when the specific scene has not been detected. Alternatively, the exclusion target determination process may be performed directly after the classification process. Specifically, when detecting a specific object, the exclusion target determination process may be performed on the image of an area classified as the abnormal area.

According to the fourth embodiment, it is possible to suppress a situation in which an object that is classified as the abnormal area, but should not be enhanced (e.g., water supply area) is enhanced, by performing the enhancement process only on a structure that does not fall under the exclusion target, and has been classified as the abnormal area. It is possible to assist in a qualitative lesion/non-lesion diagnosis by thus performing the enhancement process while excluding a structure other than a mucous membrane that may be classified as a lesion due to a difference from a normal tissue surface shape.

### 6. First Classification Method

### 6.1. Classification Section

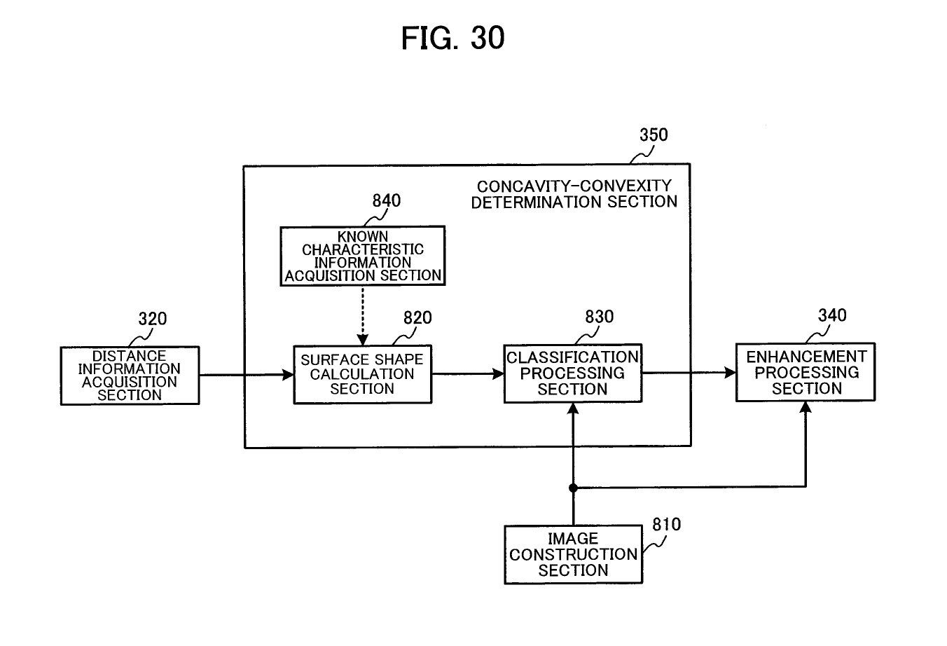

The classification process performed by the concavity-convexity determination section 350 according to the third and fourth embodiments is described in detail below. FIG. 30 illustrates a detailed configuration example of the concavity-convexity determination section 350. The concavity-convexity determination section 350 includes a known characteristic information acquisition section 840, the surface shape calculation section 820, and the classification processing section 830.

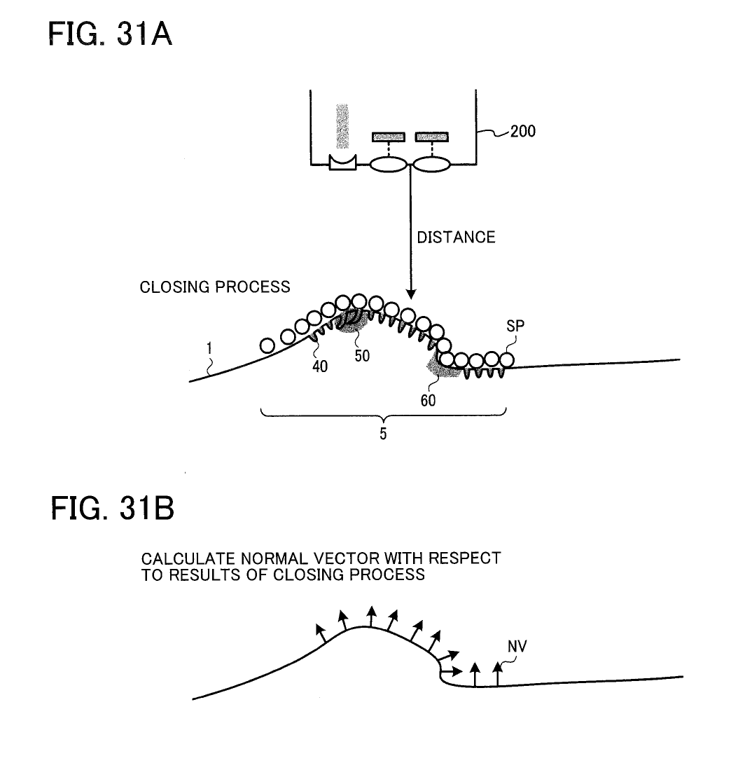

The operation of the concavity-convexity determination section 350 is described below taking an example in which the observation target is the large intestine. As illustrated in FIG. 31A, a polyp 5 (i.e., elevated lesion) is present on the surface 1 of the large intestine (i.e., observation target), and a normal duct 40 and an abnormal duct 50 are present in the surface layer of the mucous membrane of the polyp 5. A recessed lesion 60 (in which the ductal structure has disappeared) is present at the base of the polyp 5. As illustrated in FIG. 24B, when the polyp 5 is viewed from above, the normal duct 40 has an approximately circular shape, and the abnormal duct 50 has a shape differing from that of the normal duct 40.

The surface shape calculation section 820 performs the closing process or the adaptive low-pass filtering process on the distance information (e.g., distance map) input from the distance information acquisition section 320 to extract a structure having a size equal to or larger than that of a given structural element. The given structural element is the classification target ductal structure (pit pattern) formed on the surface 1 of the observation target part.

Specifically, the known characteristic information acquisition section 840 acquires structural element information as the known characteristic information, and outputs the structural element information to the surface shape calculation section 820. The structural element information is size information that is determined by the optical magnification of the imaging section 200, and the size (width information) of the ductal structure to be classified from the surface structure of the surface 1. Specifically, the optical magnification is determined corresponding to the distance to the object, and the size on the image of the ductal structure within the image captured at the distance to the object is acquired as the structural element information by performing a size adjustment process using the optical magnification.

For example, the control section 302 included in the processor section 300 stores a standard size of a ductal structure, and the known characteristic information acquisition section 840 acquires the standard size from the control section 302, and performs the size adjustment process using the optical magnification. Specifically, the control section 302 determines the observation target part based on the scope ID information input from the memory 211 of the imaging section 200. For example, when the imaging section 200 is an upper gastrointestinal scope, the observation target part is determined to be the gullet, the stomach, or the duodenum. When the imaging section 200 is a lower gastrointestinal scope, the observation target part is determined to be the large intestine. A standard duct size corresponding to each observation target part is stored in the control section 302 in advance. When the external I/F section 500 includes a switch that can be operated by the user for selecting the observation target part, the user may select the observation target part by operating the switch, for example.

The surface shape calculation section 820 adaptively generates surface shape calculation information based on the input distance information, and calculates the surface shape information about the object using the surface shape calculation information. The surface shape information represents the normal vector NV illustrated in FIG. 31B, for example. The details of the surface shape calculation information are described later. For example, the surface shape calculation information may be the morphological kernel size (i.e., the size of the structural element) that is adapted to the distance information at the attention position on the distance map, or may be the low-pass characteristics of a filter that is adapted to the distance information. Specifically, the surface shape calculation information is information that adaptively changes the characteristics of a nonlinear or linear low-pass filter corresponding to the distance information.

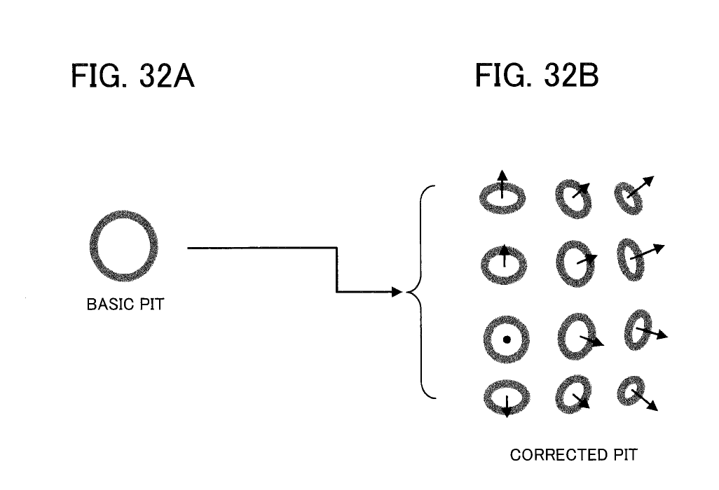

The surface shape information thus generated is input to the classification processing section 830 together with the distance map. As illustrated in FIGS. 32A and 32B, the classification processing section 830 generates a corrected pit (classification reference) from a basic pit corresponding to the three-dimensional shape of the surface of tissue captured within the captured image. The basic pit is generated by modeling a normal ductal structure for classifying a ductal structure. The basic pit is a binary image, for example. The terms “basic pit” and “corrected pit” are used since the pit pattern is the classification target. Note that the terms “basic pit” and “corrected pit” can respectively be replaced by the terms “reference pattern” and “corrected pattern” having a broader meaning.

The classification processing section 830 performs the classification process using the generated classification reference (corrected pit). Specifically, the image output from the image construction section 810 is input to the classification processing section 830. The classification processing section 830 determines the presence or absence of the corrected pit within the captured image using a known pattern matching process, and outputs a classification map (in which the classification areas are grouped) to the enhancement processing section 340. The classification map is a map in which the captured image is classified into an area that includes the corrected pit and an area other than the area that includes the corrected pit. For example, the classification map is a binary image in which “1” is assigned to pixels included in an area that includes the corrected pit, and “0” is assigned to pixels included in an area other than the area that includes the corrected pit.

The image (having the same size as that of the classification image) output from the image construction section 810 is input to the enhancement processing section 340. The enhancement processing section 340 performs the enhancement process on the image output from the image construction section 810 using the information that represents the classification results.

### 6.2. Surface Shape Calculation Section

The process performed by the surface shape calculation section 820 is described below with reference to FIGS. 31A and 31B.

FIG. 31A is a cross-sectional view illustrating the surface 1 of the object and the imaging section 200 taken along the optical axis of the imaging section 200. FIG. 31A schematically illustrates a state in which the surface shape is calculated using the morphological process (closing process). The radius of the sphere SP (structural element) used for the closing process is set to be equal to or more than twice the size of the classification target ductal structure (surface shape calculation information), for example. The size of the ductal structure has been adjusted to the size within the image corresponding to the distance to the object corresponding to each pixel (see above).

It is possible to extract the three-dimensional surface shape of the smooth surface 1 without extracting the minute concavities-convexities of the normal duct 40, the abnormal duct 50, and the duct disappearance area 60 by utilizing the sphere SP having such a size. This makes it possible to reduce a correction error as compared with the case of correcting the basic pit using the surface shape in which the minute concavities-convexities remain.

FIG. 31B is a cross-sectional view illustrating the surface of the tissue after the closing process has been performed. FIG. 31B illustrates the results of a normal vector (NV) calculation process performed on the surface of the tissue. The normal vector NV is used as the surface shape information. Note that the surface shape information is not limited to the normal vector NV. The surface shape information may be the curved surface illustrated in FIG. 31B, or may be another piece of information that represents the surface shape.

Specifically, the known characteristic information acquisition section 840 acquires the size (e.g., the width in the longitudinal direction) of the duct of tissue as the known characteristic information, and determines the radius (corresponding to the size of the duct within the image) of the sphere SP used for the closing process. In this case, the radius of the sphere SP is set to be larger than the size of the duct within the image. The surface shape calculation section 820 can extract only the desired surface shape by performing the closing process using the sphere SP.

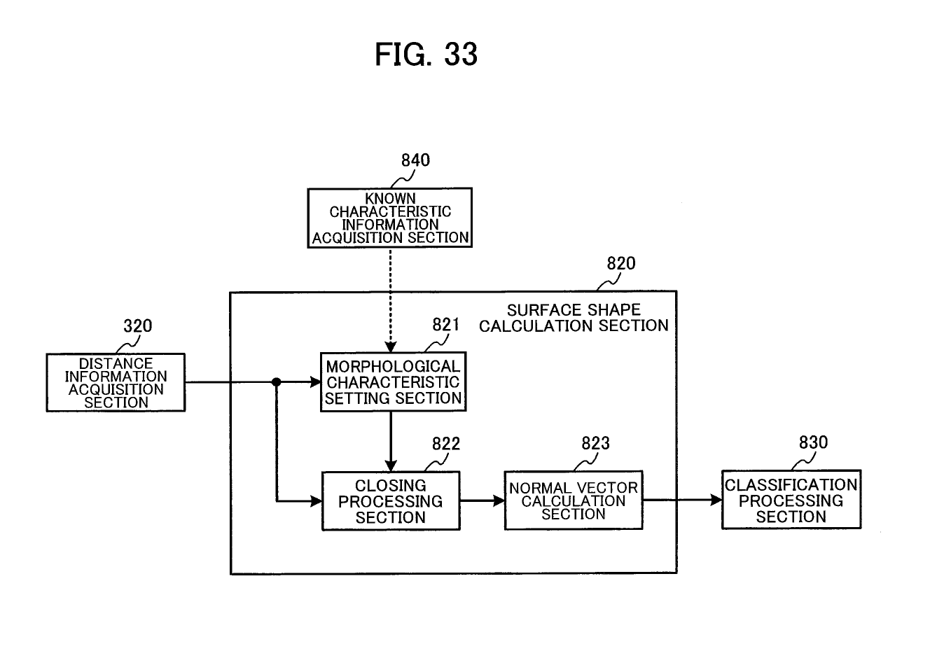

FIG. 33 illustrates a detailed configuration example of the surface shape calculation section 820. The surface shape calculation section 820 includes a morphological characteristic setting section 821, a closing processing section 822, and a normal vector calculation section 823.

The size (e.g., the width in the longitudinal direction) of the duct of tissue (i.e., known characteristic information) is input to the morphological characteristic setting section 821 from the known characteristic information acquisition section 840. The morphological characteristic setting section 821 determines the surface shape calculation information (e.g., the radius of the sphere SP used for the closing process) based on the size of the duct and the distance map.

The information about the radius of the sphere SP thus determined is input to the closing processing section 822 as a radius map having the same number of pixels as that of the distance map, for example. The radius map is a map in which the information about the radius of the sphere SP corresponding to each pixel is linked to each pixel. The closing processing section 822 performs the closing process while changing the radius of the sphere SP on a pixel basis using the radius map, and outputs the processing results to the normal vector calculation section 823.

The distance map obtained by the closing process is input to the normal vector calculation section 823. The normal vector calculation section 323 defines a plane using three-dimensional information (e.g., the coordinates of the pixel and the distance information at the coordinates) about the attention sampling position and two sampling positions adjacent thereto on the distance map, and calculates the normal vector to the defined plane. The normal vector calculation section 323 outputs the calculated normal vector to the classification processing section 830 as a normal vector map that is identical with the distance map as to the number of sampling points.

Note that the surface shape calculated in connection with the third and fourth embodiments basically differs from the concavities-convexities extracted in connection with the first and second embodiments. Specifically, while the extracted concavity-convexity information is information about minute concavities-convexities excluding global concavities-convexities (FIG. 10B) (see FIG. 10C), the surface shape information is information about global concavities-convexities obtained by smoothing a ductal structure (see FIG. 31B).

The morphological process performed when calculating the surface shape and the morphological process performed when calculating global concavities-convexities in order to obtain the extracted concavity-convexity information (e.g., FIG. 9B) differ in the scale of the smoothing target structure and the size of the structural element. Therefore, these morphological processes are basically implemented by different processing sections. For example, when extracting concavities-convexities, the extraction target is a groove or a polyp, and a structural element having a size corresponding to the size of the extraction target is used. When calculating the surface shape, a minute pit pattern that can be observed by close (zoom) observation is smoothed. Therefore, the size of the structural element is smaller than that of the structural element used when extracting concavities-convexities. Note that the above morphological processes may be implemented by a common processing section when a structural element having an almost identical size is used, for example.

### 6.3. Classification Processing Section

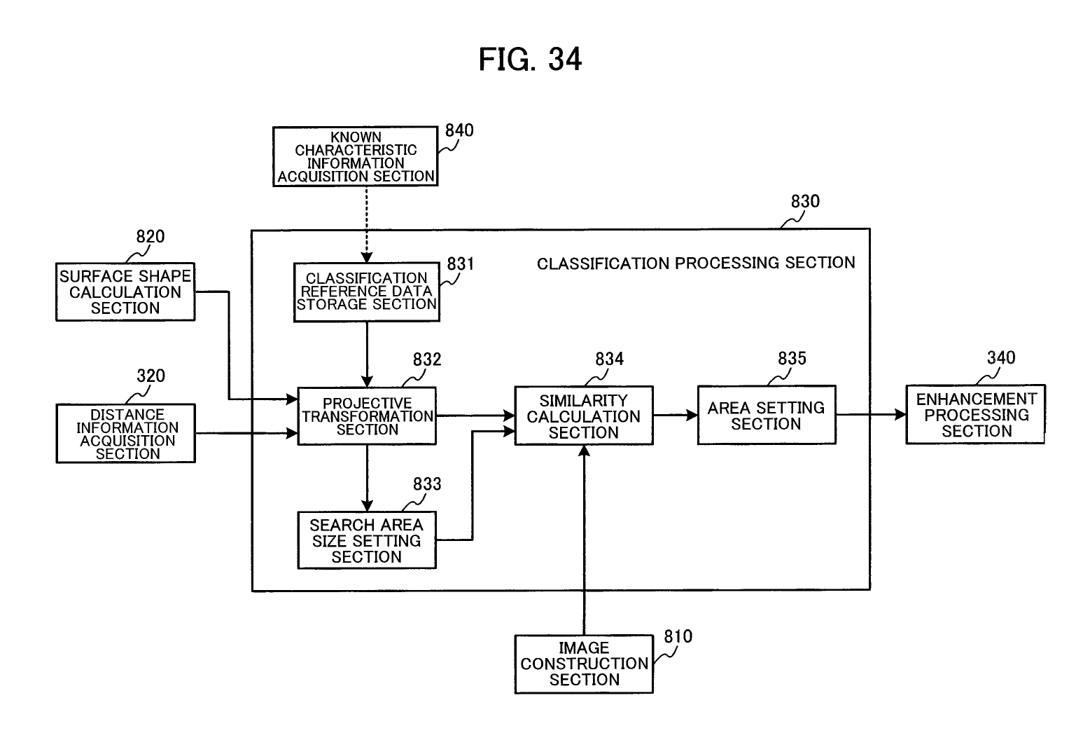

FIG. 34 illustrates a detailed configuration example of the classification processing section 830. The classification processing section 830 includes a classification reference data storage section 831, a projective transformation section 832, a search area size setting section 833, a similarity calculation section 834, and an area setting section 835.

The classification reference data storage section 831 stores the basic pit obtained by modeling the normal duct exposed on the surface of the tissue (see FIG. 32A). The basic pit is a binary image having a size corresponding to the size of the normal duct captured at a given distance. The classification reference data storage section 831 outputs the basic pit to the projective transformation section 832.

The distance map output from the distance information acquisition section 320, the normal vector map output from the surface shape calculation section 820, and the optical magnification output from the control section 302 are input to the projective transformation section 832. The projective transformation section 832 extracts the distance information corresponding to the attention sampling position from the distance map, and extracts the normal vector at the sampling position corresponding thereto from the normal vector map. The projective transformation section 832 subjects the basic pit to projective transformation using the normal vector, and performs a magnification correction process corresponding to the optical magnification to generate a corrected pit. The projective transformation section 832 outputs the corrected pit to the similarity calculation section 834 as the classification reference, and outputs the size of the corrected pit to the search area size setting section 833.

The search area size setting section 833 sets an area having a size twice the size of the corrected pit to be a search area used for a similarity calculation process, and outputs the information about the search area to the similarity calculation section 834.

The similarity calculation section 834 receives the corrected pit at the attention sampling position from the projective transformation section 832, and receives the search area corresponding to the corrected pit from the search area size setting section 833. The similarity calculation section 834 extracts the image of the search area from the image input from the image construction section 810.

The similarity calculation section 834 performs a high-pass filtering process or a band-pass filtering process on the extracted image of the search area to remove a low-frequency component, and performs a binarization process on the resulting image to generate a binary image of the search area. The similarity calculation section 834 performs a pattern matching process on the binary image of the search area using the corrected pit to calculate a correlation value, and outputs the peak position of the correlation value and a maximum correlation value map to the area setting section 835. For example, the correlation value is the sum of absolute differences, and the maximum correlation value is the minimum value of the sum of absolute differences.

Note that the correlation value may be calculated using a phase-only correlation (POC) method or the like. Since rotation and a change in magnification become invariable when using the POC method, it is possible to improve the correlation calculation accuracy.

The area setting section 835 calculates an area for which the sum of absolute differences is equal to or less than a given threshold value T based on the maximum correlation value map input from the similarity calculation section 834, and calculates the three-dimensional distance between the position within the calculated area that corresponds to the maximum correlation value and the position within the adjacent search range that corresponds to the maximum correlation value. When the calculated three-dimensional distance is included within a given error range, the area setting section 835 groups an area including the maximum correlation position as a normal area to generate a classification map. The area setting section 835 outputs the generated classification map to the enhancement processing section 340.

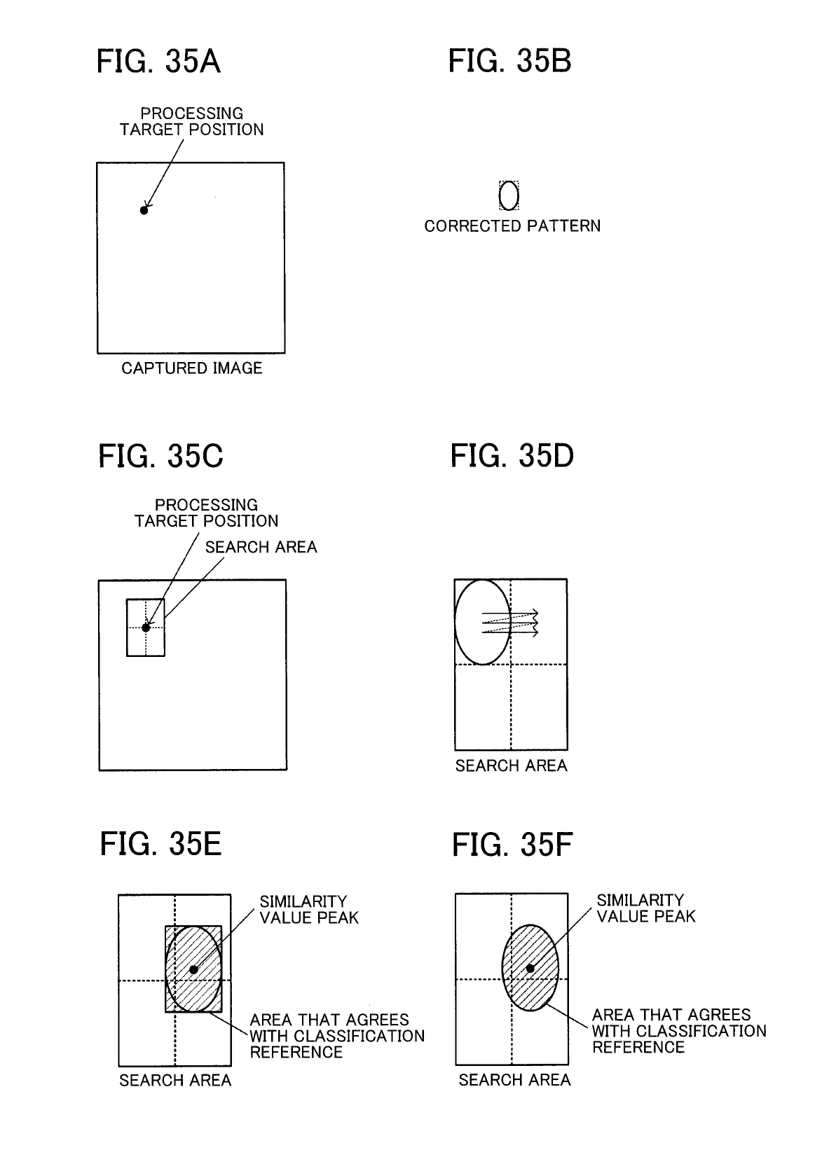

FIGS. 35A to 35F illustrate a specific example of the classification process. As illustrated in FIG. 35A, one position within the image is set to be the processing target position. The projective transformation section 832 acquires a corrected pattern at the processing target position by deforming the reference pattern based on the surface shape information at the processing target position (see FIG. 35B). The search area size setting section 833 sets the search area (e.g., an area having a size twice the size of the corrected pit pattern) around the processing target position from the acquired corrected pattern (see FIG. 35C).

The similarity calculation section 834 performs the matching process on the captured structure and the corrected pattern within the search area (see FIG. 35D). When the matching process is performed on a pixel basis, the similarity is calculated on a pixel basis. The area setting section 835 specifies a pixel that corresponds to the peak of the similarity within the search area (see FIG. 35E), and determines whether or not the similarity at the specified pixel is equal to or larger than a given threshold value. When the similarity at the specified pixel is equal to or larger than the threshold value (i.e., when the corrected pattern has been detected within the area having the size of the corrected pattern based on the peak position (the center of the corrected pattern is set to be the reference position in FIG. 35E)), it is determined that the area agrees with the reference pattern.

Note that the inside of the shape that represents the corrected pattern may be determined to be the area that agrees with the classification reference (see FIG. 35F). Various other modifications may also be made. When the similarity at the specified pixel is less than the threshold value, it is determined that a structure that matches the reference pattern is not present in the area around the processing target position. An area (0, 1, or a plurality of areas) that agrees with the reference pattern, and an area other than the area that agrees with the reference pattern are set within the captured image by performing the above process at each position within the image. When a plurality of areas agree with the reference pattern, overlapping areas and contiguous areas among the plurality of areas are integrated to obtain the classification results. Note that the classification process based on the similarity described above is only an example. The classification process may be performed using another method. The similarity may be calculated using various known methods that calculate the similarity between images or the difference between images, and detailed description thereof is omitted.

According to the above embodiment, the concavity-convexity determination section 350 includes the surface shape calculation section 820 that calculates the surface shape information about the object based on the distance information and the known characteristic information, and the classification processing section 830 that generates the classification reference based on the surface shape information, and performs the classification process that utilizes the generated classification reference.

This makes it possible to adaptively generate the classification reference based on the surface shape represented by surface shape information, and perform the classification process. A decrease in the accuracy of the classification process due to the surface shape may occur due to deformation of the structure within the captured image caused by the angle formed by the optical axis direction of the imaging section 200 and the surface of the object, for example. The method according to the above embodiment makes it possible to accurately perform the classification process even in such a situation.

The known characteristic information acquisition section 840 may acquire the reference pattern that corresponds to the structure of the object in a given state as the known characteristic information, and the classification processing section 830 may generate the corrected pattern as the classification reference, and perform the classification process using the generated classification reference, the corrected pattern being acquired by performing a deformation process based on the surface shape information on the reference pattern.

This makes it possible to accurately perform the classification process even when the structure of the object is captured in a deformed state due to the surface shape. Specifically, a circular ductal structure may be captured in a variously deformed state (see FIG. 1B, for example). It is possible to appropriately detect and classify the pit pattern even in a deformed area by generating an appropriate corrected pattern (corrected pit in FIG. 32B) from the reference pattern (basic pit in FIG. 32A) corresponding to the surface shape, and utilizing the generated corrected pattern as the classification reference.

The known characteristic information acquisition section 840 may acquire the reference pattern that corresponds to the structure of the object in a normal state as the known characteristic information.

This makes it possible to implement the classification process that classifies the captured image into a normal area and an abnormal area. The term “abnormal area” refers to an area that is considered to be a lesion when using a medical endoscope, for example. Since it is considered that the user pays attention to such an area, a situation in which the attention area is missed can be suppressed by appropriately classifying the captured image.

The object may include a global three-dimensional structure, and a local concavity-convexity structure that is more local than the global three-dimensional structure, and the surface shape calculation section 820 may calculate the surface shape information by extracting the global three-dimensional structure among the global three-dimensional structure and the local concavity-convexity structure included in the object from the distance information.

This makes it possible to calculate the surface shape information from the global structure when the structures of the object are classified into a global structure and a local structure. Deformation of the reference pattern within the captured image predominantly occurs due to a global structure that is larger than the reference pattern. Therefore, an accurate classification process can be implemented by calculating the surface shape information from the global three-dimensional structure.

### 7. Second Classification Method

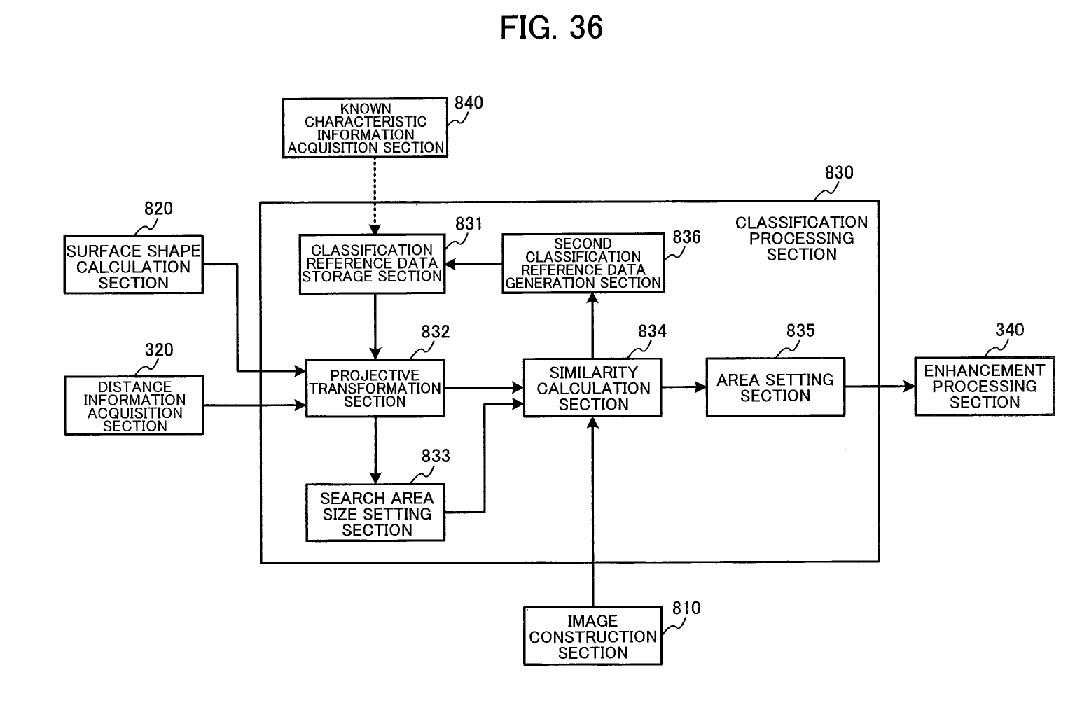

FIG. 36 illustrates a detailed configuration example of the classification processing section 830 according to the second classification method. The classification processing section 830 includes a classification reference data storage section 831, a projective transformation section 832, a search area size setting section 833, a similarity calculation section 834, an area setting section 835, and a second classification reference data generation section 836. Note that the same elements as those described above in connection with the first classification method are indicated by the same reference symbols, and description thereof is appropriately omitted.

The second classification method differs from the first classification method in that the basic pit (classification reference) is provided corresponding to the normal duct and the abnormal duct, a pit is extracted from the actual captured image, and used as second classification reference data (second reference pattern), and the similarity is calculated based on the second classification reference data.

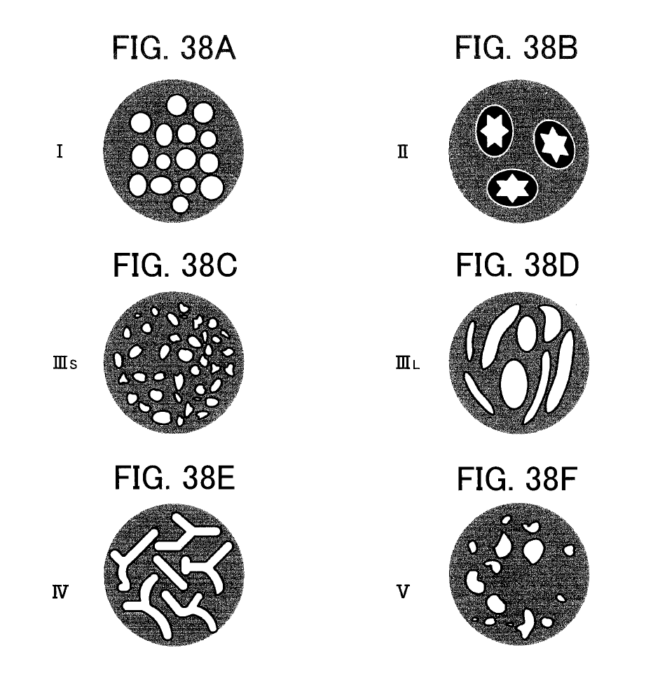

As illustrated in FIGS. 38A to 38F, the shape of a pit pattern on the surface of tissue changes corresponding to the state (normal state or abnormal state), the stage of lesion progression (abnormal state), and the like. For example, the pit pattern of a normal mucous membrane has an approximately circular shape (see FIG. 38A). The pit pattern has a complex shape (e.g., star-like shape (see FIG. 38B) or tubular shape (see FIGS. 38C and 38D) when a lesion has advanced, and may disappear (see FIG. 38F) when the lesion has further advanced. Therefore, it is possible to determine the state of the object by storing these typical patterns as a reference pattern, and determining the similarity between the surface of the object captured within the captured image and the reference pattern, for example.

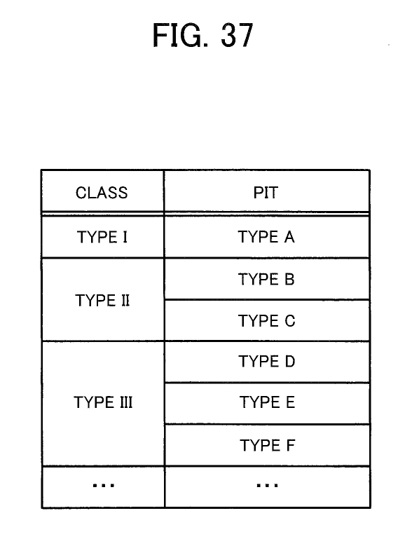

The differences from the first classification method are described in detail below. A plurality of pits including the basic pit corresponding to the normal duct (see FIG. 37) are stored in the classification reference data storage section 831, and output to the projective transformation section 832. The process performed by the projective transformation section 832 is the same as described above in connection with the first classification method. Specifically, the projective transformation section 832 performs the projective transformation process on each pit stored in the classification reference data storage section 831, and outputs the corrected pits corresponding to a plurality of classification types to the search area size setting section 833 and the similarity calculation section 834.

The similarity calculation section 834 generates the maximum correlation value map corresponding to each corrected pit. Note that the maximum correlation value map is not used to generate the classification map (i.e., the final output of the classification process), but is output to the second classification reference data generation section 836, and used to generate additional classification reference data.

The second classification reference data generation section 836 sets the pit image at a position within the image for which the similarity calculation section 834 has determined that the similarity is high (i.e., the absolute difference is equal to or smaller than a given threshold value) to be the classification reference. This makes it possible to implement a more optimum and accurate classification (determination) process since the pit extracted from the actual image is used as the classification reference instead of using a typical pit model provided in advance.

More specifically, the maximum correlation value map (corresponding to each type) output from the similarity calculation section 834, the image output from the image construction section 810, the distance map output from the distance information acquisition section 320, the optical magnification output from the control section 302, and the duct size (corresponding to each type) output from the known characteristic information acquisition section 840 are input to the second classification reference data generation section 836. The second classification reference data generation section 836 extracts the image data corresponding to the maximum correlation value sampling position (corresponding to each type) based on the distance information at the maximum correlation value sampling position, the size of the duct, and the optical magnification.

The second classification reference data generation section 836 acquires a grayscale image (that cancels the difference in brightness) obtained by removing a low-frequency component from the extracted (actual) image, and outputs the grayscale image to the classification reference data storage section 831 as the second classification reference data together with the normal vector and the distance information. The classification reference data storage section 831 stores the second classification reference data and relevant information. The second classification reference data having a high correlation with the object has thus been collected corresponding to each type.

Note that the second classification reference data includes the effects of the angle formed by the optical axis direction of the imaging section 200 and the surface of the object, and the effects of deformation (change in size) depending on the distance from the imaging section 200 to the surface of the object. Therefore, the second classification reference data generation section 836 may generate the second classification reference data after performing a process that cancels these effects.

Specifically, the results of a deformation process (projective transformation process and scaling process) performed on the grayscale image so as to achieve a state in which the image is captured at a given distance from a given reference direction may be used as the second classification reference data.

After the second classification reference data has been generated, the projective transformation section 832, the search area size setting section 833, and the similarity calculation section 834 perform the process on the second classification reference data. Specifically, the projective transformation process is performed on the second classification reference data to generate a second corrected pattern, and the process described above in connection with the first classification method is performed using the generated second corrected pattern as the classification reference.

Note that the basic pit corresponding to the abnormal duct used in connection with the second classification method is not normally point-symmetrical. Therefore, it is desirable that the similarity calculation section 834 calculate the similarity (when using the corrected pattern or the second corrected pattern) by performing the rotation-invariant phase-only correction (POC).

The area setting section 835 generates the classification map in which the pits are grouped on a class basis (type I, type II, . . . ) (see FIG. 37), or generates the classification map in which the pits are grouped on a type basis (type A, type B, . . . ) (see FIG. 37). Specifically, the area setting section 835 generates the classification map of an area in which a correlation is obtained by the corrected pit classified as the normal duct, and generates the classification map of an area in which a correlation is obtained by the corrected pit classified as the abnormal duct on a class basis and a type basis. The area setting section 835 synthesizes these classification maps to generate a synthesized classification map (multi-valued image). In this case, the overlapping area of the areas in which a correlation is obtained corresponding to each class may be set to an unclassified area, or may be set to the type with a higher malignant level. The area setting section 835 outputs the synthesized classification map to the enhancement processing section 340.

The enhancement processing section 340 performs the luminance or color enhancement process based on the classification map (multi-valued image), for example.

According to the fourth embodiment, the known characteristic information acquisition section 840 acquires the reference pattern that corresponds to the structure of the object in an abnormal state as the known characteristic information.

This makes it possible to acquire a plurality of reference patterns (see FIG. 37), generate the classification reference using the plurality of reference patterns, and perform the classification process, for example. Specifically, the state of the object can be finely classified by performing the classification process using the typical patterns illustrated in FIGS. 38A to 38F as the reference pattern.

The known characteristic information acquisition section 840 may acquire the reference pattern that corresponds to the structure of the object in a given state as the known characteristic information, and the classification processing section 830 may perform the deformation process based on the surface shape information on the reference pattern to acquire the corrected pattern, calculate the similarity between the structure of the object captured within the captured image and the corrected pattern at each position within the captured image, and acquire a second reference pattern candidate based on the calculated similarity. The classification processing section 830 may generate the second reference pattern as a new reference pattern based on the acquired second reference pattern candidate and the surface shape information, perform the deformation process based on the surface shape information on the second reference pattern to generate the second corrected pattern as the classification reference, and perform the classification process using the generated classification reference.

This makes it possible to generate the second reference pattern based on the captured image, and perform the classification process using the second reference pattern. Since the classification reference can be generated from the object captured within the captured image, the classification reference sufficiently reflects the characteristics of the processing target object, and it is possible to improve the accuracy of the classification process as compared with the case of directly using the reference pattern acquired as the known characteristic information.

The image processing device, the endoscope image processing device (image processing section 301), and the like according to the embodiments of the invention may include a processor and a memory. The processor may be a central processing unit (CPU), for example. Note that the processor is not limited to a CPU. Various other processors such as a graphics processing unit (GPU) or a digital signal processor (DSP) may also be used. The processor may be a hardware circuit that includes an ASIC. The memory stores a computer-readable instruction. Each section of the image processing device, the endoscope image processing device (image processing section 301), and the like according to the embodiments of the invention is implemented by causing the processor to execute the instruction. The memory may be a semiconductor memory (e.g., SRAM or DRAM), a register, a hard disk, or the like. The instruction may be an instruction included in an instruction set included in a program, or may be an instruction that causes a hardware circuit of the processor to operate.

### 8. Processing by Software

Some or most of the processes performed by the image processing section 301 according to the embodiments of the invention may be implemented by a program. In this case, the image processing section 301 according to the embodiments of the invention is implemented by causing a processor (e.g., CPU) to execute a program. Specifically, a program stored in an information storage device is read, and executed by a processor (e.g., CPU). The information storage device (computer-readable device) stores a program, data, and the like. The function of the information storage device may be implemented by an optical disk (e.g., DVD or CD), a hard disk drive (HDD), a memory (e.g., memory card or ROM), or the like. The processor (e.g., CPU) performs various processes according to the embodiments of the invention based on the program (data) stored in the information storage device. Specifically, a program that causes a computer (i.e., a device that includes an operation section, a processing section, a storage section, and an output section) to function as each section according to the embodiments of the invention (i.e., a program that causes a computer to execute the process implemented by each section) is stored in the information storage device. Note that an image processing method (i.e., a method for operating or controlling an image processing device) may be implemented by an image processing device (hardware), or may be implemented by causing a CPU to execute a program that describes the process of the image processing method.

Although only some embodiments of the invention and the modifications thereof have been described in detail above, those skilled in the art will readily appreciate that many modifications are possible in the embodiments and the modifications thereof without materially departing from the novel teachings and advantages of the invention. A plurality of elements described in connection with the above embodiments and the modifications thereof may be appropriately combined to implement various configurations. For example, some elements may be omitted from the elements described in connection with the above embodiments and the modifications thereof. Some of the elements described in connection with different embodiments and modifications thereof may be appropriately combined. Specifically, various modifications and applications are possible without materially departing from the novel teachings and advantages of the invention. Any term cited with a different term having a broader meaning or the same meaning at least once in the specification and the drawings can be replaced by the different term in any place in the specification and the drawings.

What is claimed is:

1. An endoscope image processing device comprising:
an image acquisition section that acquires a captured image that includes an image of an object;
a distance information acquisition section that acquires distance information based on a distance from an imaging section to the object when the imaging section captured the captured image;
a concavity-convexity determination section that performs a concavity-convexity determination process based on the distance information, and known characteristic information that represents known characteristics relating to a structure of the object, the concavity-convexity determination process determining a concavity-convexity part of the object that agrees with the characteristics specified by the known characteristic information;
a mucous membrane determination section that determines a mucous membrane area within the captured image, the mucous membrane area being an area of a mucous membrane; and
an enhancement processing section that performs an enhancement process on the mucous membrane area determined by the mucous membrane determination section based on information about the concavity-convexity part determined by the concavity-convexity determination process,
the concavity-convexity determination section excluding a structure that is more global than a local concavity-convexity structure having a desired size from the distance information based on the known characteristic information to extract the local concavity-convexity structure having the desired size as the concavity-convexity part.

2. The endoscope image processing device as defined in claim 1,
the mucous membrane determination section determining an area for which a feature quantity based on a pixel value of the captured image satisfies a given condition that corresponds to the mucous membrane, to be the mucous membrane area.

3. The endoscope image processing device as defined in claim 2,
the mucous membrane determination section determining an area for which color information that represents the feature quantity satisfies the given condition relating to a color of the mucous membrane, to be the mucous membrane area.

4. The endoscope image processing device as defined in claim 1, further comprising:
a concavity-convexity information acquisition section that extracts the concavity-convexity part of the object that agrees with the characteristics specified by the known characteristic information from the distance information as extracted concavity-convexity information based on the distance information and the known characteristic information,
the mucous membrane determination section determining an area for which the extracted concavity-convexity information agrees with concavity-convexity characteristics represented by the known characteristic information, to be the mucous membrane area.

5. The endoscope image processing device as defined in claim 4,
the mucous membrane determination section acquiring dimensional information that represents at least one of a width and a depth of a concavity of the object as the known characteristic information, extracting the concavity included in the extracted concavity-convexity information that agrees with characteristics specified by the dimensional information, and determining a concavity area within the captured image that corresponds to the extracted concavity, and an area situated in the vicinity of the concavity area, to be the mucous membrane area.

6. The endoscope image processing device as defined in claim 5,
the mucous membrane determination section detecting a pixel situated outside the concavity area as the area situated in the vicinity of the concavity area when a difference between the distance to the object corresponding to a pixel within the concavity area and the distance to the object corresponding to the pixel situated outside the concavity area is shorter than a given distance.

7. The endoscope image processing device as defined in claim 1,
the enhancement processing section performing the enhancement process using an enhancement level that continuously changes at a boundary between the mucous membrane area and an area other than the mucous membrane area.

8. The endoscope image processing device as defined in claim 1, further comprising:
a concavity-convexity information acquisition section that extracts the concavity-convexity part of the object that agrees with the characteristics specified by the known characteristic information from the distance information as extracted concavity-convexity information based on the distance information and the known characteristic information,
the enhancement processing section performing the enhancement process that enhances a specific color corresponding to the distance to the object represented by the extracted concavity-convexity information.

9. The endoscope image processing device as defined in claim 1,
the concavity-convexity determination section including a concavity-convexity information acquisition section that extracts the concavity-convexity part of the object that agrees with the characteristics specified by the known characteristic information from the distance information as extracted concavity-convexity information based on the distance information and the known characteristic information, and
the concavity-convexity determination section performing a process that extracts the concavity-convexity part as the concavity-convexity determination process.

10. The endoscope image processing device as defined in claim 1,
the concavity-convexity determination section including:
a surface shape calculation section that calculates surface shape information about the object based on the distance information and the known characteristic information; and
a classification processing section that generates a classification reference based on the surface shape information, and performs a classification process that utilizes the generated classification reference, and
the concavity-convexity determination section performing the classification process that utilizes the classification reference as the concavity-convexity determination process.

11. The endoscope image processing device as defined in claim 10,
the concavity-convexity determination section performing the classification process on the mucous membrane area determined by the mucous membrane determination section.

12. The endoscope image processing device as defined in claim 10,
the mucous membrane determination section performing a process that determines the mucous membrane area on the object that has been classified as a specific class by the classification process.

13. The endoscope image processing device as defined in claim 12,
the classification processing section determining whether or not a pixel or an area within the captured image agrees with a classification reference that corresponds to a normal structure to classify the pixel or the area as a normal area or a non-normal area, and
the mucous membrane determination section performing a process that determines the mucous membrane area on the pixel or the area that has been classified as the non-normal area.

14. An endoscope image processing device comprising:
an image acquisition section that acquires a captured image that includes an image of an object;
a distance information acquisition section that acquires distance information based on a distance from an imaging section to the object when the imaging section captured the captured image;
a concavity-convexity determination section that performs a concavity-convexity determination process based on the distance information, and known characteristic information that represents known characteristics relating to a structure of the object, the concavity-convexity determination process determining a concavity-convexity part of the object that agrees with the characteristics specified by the known characteristic information;
an exclusion target determination section that determines an exclusion target area within the captured image, the exclusion target area being an area of an exclusion target; and
an enhancement processing section that performs an enhancement process on the captured image based on information about the concavity-convexity part determined by the concavity-convexity determination process, while omitting or suppressing the enhancement process on the exclusion target area determined by the exclusion target determination section,
the concavity-convexity determination section excluding a structure that is more global than a local concavity-convexity structure having a desired size from the distance information based on the known characteristic information to extract the local concavity-convexity structure having the desired size as the concavity-convexity part.

15. The endoscope image processing device as defined in claim 14,
the exclusion target determination section determining an area for which a feature quantity based on a pixel value of the captured image satisfies a given condition that corresponds to the exclusion target, to be the exclusion target area.

16. The endoscope image processing device as defined in claim 15,
the exclusion target determination section determining an area for which color information that represents the feature quantity satisfies the given condition relating to a color of the exclusion target, to be the exclusion target area.

17. The endoscope image processing device as defined in claim 16,
the given condition being a condition whereby the color information belongs to a color range that corresponds to a residue, or a color range that corresponds to a treatment tool.

18. The endoscope image processing device as defined in claim 15,
the exclusion target determination section determining an area for which brightness information that represents the feature quantity satisfies the given condition relating to brightness of the exclusion target, to be the exclusion target area.

19. The endoscope image processing device as defined in claim 18,
the given condition being a condition whereby the brightness information belongs to a brightness range that corresponds to a blocked-up shadow area within the captured image, or a brightness range that corresponds to a blown-out highlight area within the captured image.

20. The endoscope image processing device as defined in claim 14,
the exclusion target determination section determining an area for which the distance information satisfies a given condition relating to a distance of the exclusion target, to be the exclusion target area.

21. The endoscope image processing device as defined in claim 20,
the exclusion target determination section determining an area in which the distance to the object represented by the distance information continuously changes, to be the exclusion target area.

22. The endoscope image processing device as defined in claim 21,
the exclusion target determination section determining that a treatment tool has been inserted when a number of pixels within a forceps channel neighborhood area within the captured image at which the distance to the object is shorter than a given distance is equal to or larger than a given number, setting the pixels within the forceps channel neighborhood area at which the distance to the object is shorter than the given distance to be the exclusion target area when it has been determined that the treatment tool has been inserted, and determining a pixel that is situated adjacent to the pixels within the exclusion target area to be the exclusion target area when a difference between the distance to the object at the pixels within the exclusion target area and the distance to the object at the pixel that is situated adjacent to the pixels within the exclusion target area is shorter than a given distance.

23. The endoscope image processing device as defined in claim 14, further comprising:
a concavity-convexity information acquisition section that extracts the concavity-convexity part of the object that agrees with the characteristics specified by the known characteristic information from the distance information as extracted concavity-convexity information based on the distance information and the known characteristic information,
the exclusion target determination section determining an area for which the extracted concavity-convexity information satisfies a given condition relating to concavities and convexities that correspond to the exclusion target, to be the exclusion target area.

24. The endoscope image processing device as defined in claim 23,
the given condition being a condition that represents a flat area of the object.

25. The endoscope image processing device as defined in claim 14,
the exclusion target determination section including a control information reception section that receives control information about an endoscope apparatus, and
the exclusion target determination section determining the captured image to be the exclusion target area when the control information received by the control information reception section is given control information that corresponds to an exclusion target scene that is the exclusion target.

26. The endoscope image processing device as defined in claim 25,
the given control information being information that instructs to supply water to the object, or information that instructs to enable an IT knife.

27. The endoscope image processing device as defined in claim 14,
the enhancement processing section performing the enhancement process using an enhancement level that continuously changes at a boundary of the exclusion target area.

28. The endoscope image processing device as defined in claim 14,
the exclusion target being an object other than a mucous membrane.

29. The endoscope image processing device as defined in claim 28,
the object other than the mucous membrane being a residue, a treatment tool, a blocked-up shadow area, or a blown-out highlight area.

30. The endoscope image processing device as defined in claim 14,
the concavity-convexity determination section including a concavity-convexity information acquisition section that extracts the concavity-convexity part of the object that agrees with the characteristics specified by the known characteristic information from the distance information as extracted concavity-convexity information based on the distance information and the known characteristic information, and
the concavity-convexity determination section performing a process that extracts the concavity-convexity part as the concavity-convexity determination process.

31. The endoscope image processing device as defined in claim 14,
the concavity-convexity determination section including:
a surface shape calculation section that calculates surface shape information about the object based on the distance information and the known characteristic information; and
a classification processing section that generates a classification reference based on the surface shape information, and performs a classification process that utilizes the generated classification reference, and
the concavity-convexity determination section performing the classification process that utilizes the classification reference as the concavity-convexity determination process.

32. An endoscope apparatus comprising the endoscope image processing device as defined in claim 1.

33. An endoscope apparatus comprising the endoscope image processing device as defined in claim 14.

34. An image processing method comprising:
acquiring a captured image that includes an image of an object;
acquiring distance information based on a distance from an imaging section to the object when the imaging section captured the captured image;
performing a concavity-convexity determination process that excludes a structure that is more global than a local concavity-convexity structure having a desired size from the distance information based on known characteristic information to extract the local concavity-convexity structure having the desired size as a concavity-convexity part of the object that agrees with characteristics specified by the known characteristic information, to determine the concavity-convexity part, the known characteristic information being information that represents known characteristics relating to a structure of the object;
determining a mucous membrane area within the captured image, the mucous membrane area being an area of a mucous membrane; and
performing an enhancement process on the determined mucous membrane area based on information about the concavity-convexity part determined by the concavity-convexity determination process.

35. An image processing method comprising:
acquiring a captured image that includes an image of an object;
acquiring distance information based on a distance from an imaging section to the object when the imaging section captured the captured image;
performing a concavity-convexity determination process that excludes a structure that is more global than a local concavity-convexity structure having a desired size from the distance information based on known characteristic information to extract the local concavity-convexity structure having the desired size as a concavity-convexity part of the object that agrees with characteristics specified by the known characteristic information, to determine the concavity-convexity part, the known characteristic information being information that represents known characteristics relating to a structure of the object;
determining an exclusion target area within the captured image, the exclusion target area being an area of an exclusion target; and
performing an enhancement process on the captured image based on information about the concavity-convexity part determined by the concavity-convexity determination process, while omitting or suppressing the enhancement process on the determined exclusion target area.

36. A non-transitory information storage device storing an image processing program that causes a computer to perform steps of:
acquiring a captured image that includes an image of an object;
acquiring distance information based on a distance from an imaging section to the object when the imaging section captured the captured image;
performing a concavity-convexity determination process that excludes a structure that is more global than a local concavity-convexity structure having a desired size from the distance information based on known characteristic information to extract the local concavity-convexity structure having the desired size as a concavity-convexity part of the object that agrees with characteristics specified by the known characteristic information, to determine the concavity-convexity part, the known characteristic information being information that represents known characteristics relating to a structure of the object;
determining a mucous membrane area within the captured image, the mucous membrane area being an area of a mucous membrane; and
performing an enhancement process on the determined mucous membrane area based on information about the concavity-convexity part determined by the concavity-convexity determination process.

37. A non-transitory information storage device storing an image processing program that causes a computer to perform steps of:
acquiring a captured image that includes an image of an object;
acquiring distance information based on a distance from an imaging section to the object when the imaging section captured the captured image;
performing a concavity-convexity determination process that excludes a structure that is more global than a local concavity-convexity structure having a desired size from the distance information based on known characteristic information to extract the local concavity-convexity structure having the desired size as a concavity-convexity part of the object that agrees with characteristics specified by the known characteristic information, to determine the concavity-convexity part, the known characteristic information being information that represents known characteristics relating to a structure of the object;
determining an exclusion target area within the captured image, the exclusion target area being an area of an exclusion target; and
performing an enhancement process on the captured image based on information about the concavity-convexity part determined by the concavity-convexity determination process, while omitting or suppressing the enhancement process on the determined exclusion target area.
